(19) United States
(12) Patent Application Publication
(10) Pub. No.: US 2021/0379148 A1
(43) Pub. Date: Dec. 9, 2021

(54) REGULATING MUSCLE PHYSIOLOGY AND ENERGY METABOLISM USING CRYM

(71) Applicant: University Of Maryland, Baltimore, Baltimore, MD (US)

(72) Inventors: Christian Kinney, Marietta, GA (US); Robert J. Bloch, Baltimore, MD (US); John McLenithan, Baltimore, MD (US); Kaila Noland, Baltimore, MD (US); Andrea O'Neill, Towson, MD (US)

(21) Appl. No.: 17/318,767

(22) Filed: May 12, 2021

Related U.S. Application Data
(60) Provisional application No. 63/023,404, filed on May 12, 2020.

(51) Int. Cl. A61K 38/17; A61K 31/11; A61K 31/203; A61K 31/167; A61K 31/565; A61K 31/568

(52) U.S. Cl. CPC A61K 38/1709; A61K 31/11; A61K 31/568; A61K 31/167; A61K 31/565; A61K 31/203

(57) ABSTRACT

Transgenic mice (Crym tg) overexpressing Crym protein in skeletal muscle were studied. Muscular functions, Ca²⁺ transients, contractile force, fatigue, and running on treadmills or wheels were not significantly altered and serum T₃ and thyroid stimulating hormone levels were unaffected although T₃ levels in tibialis anterior (TA) muscle were elevated and serum T₄ was decreased. Crym tg mice had a decreased respiratory exchange ratio, corresponding to a 13.7% increase in fat utilization. Female Crym tg mice gained weight more rapidly than controls on high fat or high simple carbohydrate diets. Machine learning algorithms revealed morphological differences between Crym tg and control soleus fibers. RNA-seq and gene ontology enrichment analysis showed a shift towards genes associated with slower muscle function and β-oxidation. Therefore, high levels of μ-crystallin are associated with greater fat metabolism. These data indicate that Crym and μ-crystallin can be used as a treatment modality for diabetes and obesity.

### CROSS-REFERENCE TO RELATED APPLICATIONS

This application claims the benefit of U.S. provisional application Ser. No. 63/023,404, filed 12 May 2020. The entire contents of this application is hereby incorporated by reference as if fully set forth herein.

### GOVERNMENT FUNDING SUPPORT

This invention was made with government support under grant no. AR057519 awarded by the National Institutes of Health. The government has certain rights in the invention.

### BACKGROUND

### 1. Field of the Invention

The present invention relates to the field of medicine, including in particular to methods for regulating energy physiology and metabolism in muscle and treatment of obesity and type 2 diabetes. Specifically, the invention relates to methods for modulating μ-crystallin (CRYM protein) levels in muscle to shift metabolism to favor the use of fat over carbohydrates as an energy source. Such methods will have utility for treating conditions such as obesity and Type 2 diabetes.

### 2. Background of the Invention

μ-Crystallin was first discovered in 1957 and called cellular thyroxine-binding protein. It was subsequently shown to bind both T3 and T4 in an NADPH-dependent manner and to have ketimine reductase activity. In 1991 μ-crystallin was characterized as a 38,000 Da polypeptide that readily forms dimers and shares structural homology with bacterial ornithine cyclodeaminase. Its structure has been solved to 1.75 Å.

The CRYM protein is highly expressed in the cerebral cortex, heart, skeletal muscle, and kidney, and has been linked to Deafness, Autosomal Dominant 40. Importantly, μ-crystallin binds T₃ with a K_{D}=0.3 nM (5) while T₃ binds thyroid hormone receptors (TR) α and β with a K_{D}=0.06 nM (T₄ binds TRs at a K_{D}=2 nM). μ-Crystallin's activity as a ketimine reductase is inhibited by T₃ and T₄ at subnanomolar levels, K₁=0.60 nM and K₁=0.75 nM, respectively. T₃ and T₄ are strong regulators of metabolism and thermogenic homeostasis. Because of this, proteins that interact with and regulate thyroid hormones may have a broad influence on integrative functions like gene expression and metabolism.

Consistent with its possible role in regulating metabolism associated with thyroid hormones, Crym knockout mice (Crym KO) fed a high fat diet (HFD) show increased fat mass by computer tomography and increased body weight compared to control mice. Furthermore, Crym KO mice show significant hypertrophy of glycolytic fast twitch type IIb muscle fibers.

### SUMMARY OF THE INVENTION

Obesity and Type 2 diabetes are major health problems. There is a great need in the art for treatments for both of these conditions. Both are closely tied to changes in metabolism in which glycolytic metabolism is enhanced, while oxidative metabolism of fats is compromised and fat storage is increased. CRYM protein (μ-crystallin), expressed in muscle, which accounts for about 50% of body mass in adults, significantly shifts energy usage in mice from glycolytic to oxidative pathways. Therefore, the invention provides methods for shifting energy toward oxidative pathways in a subject in need thereof in order to increase beta oxidation in the muscles of the subject. This forms the basis for a treatment for obesity and type 2 diabetes by increasing CRYM protein in the patient by administration of a pharmaceutical compound or a transgene.

Specifically, embodiments of the invention relate to a method of increasing expression of CRYM protein expression in a subject in need thereof, comprising administering a compound to the subject, wherein the compound is selected from the group consisting of (a) one or more chemical compounds that increases CRYM expression as described herein; and (b) a vector that expresses a CRYM protein.

In preferred embodiments of the invention, the one or more chemical compounds that increase CRYM expression are selected from the group consisting of pentanal, tretinoin, fenretinide, estradiol 3-benzoate, dihydrotestosterone and any combination thereof, and the vector comprises SEQ ID NO:2.

In certain embodiments, the subject in need suffers from a condition selected from the group consisting of a lipolytic disorder, a lipogenic disorder, a glycolytic disorder, a gluconeogenic disorder, type 2 diabetes, obesity, and a combination thereof, and preferably the subject suffers from obesity, type 2 diabetes, or a combination thereof.

In some embodiments, the administering is by intravenous, subcutaneous, or intramuscular injection or oral delivery.

Particular embodiments of the invention relate to a method of shifting energy usage from glycolytic to oxidative pathways in muscle in a subject in need thereof, comprising administering a compound to the subject, wherein the compound is selected from the group consisting of (a) one or more chemical compounds that increases CRYM expression as described herein; and (b) an AAV vector that expresses a CRYM protein.

Additional particular embodiments of the invention relate to a method of treatment of obesity and type 2 diabetes in a subject in need thereof, comprising administering a compound to the subject, wherein the compound is selected from the group consisting of (a) one or more chemical compounds that increases CRYM expression as described herein; and (b) an AAV vector that expresses a CRYM protein.

### BRIEF SUMMARY OF THE DRAWINGS

FIG. 1A through FIG. 1D. FIG. 1A shows a schematic of a plasmid construct used for transgenesis. Sequence of the plasmid insert and surrounding mouse genome are as follows. chr6:104,819,411 (intron 12 of the Cntn6 gene) base pairs 1-1466 and 5265-7073 Mouse genome; base pairs 1467-1628 Human slow troponin I enhancer (TNNI1); base pairs 1629-1664 Spacer; base pairs 1665-4037 Human skeletal actin promoter (ACTA1); base pairs 4048-4990 Crym ORF; base pairs 5036-5264 BGH polyA; base pairs 4038-4047 and 4991-5035 Plasmid backbone. FIG. 1B provides the sequence of the transgenic plasmid (SEQ ID NO:1) and the location of Crym transgene insertion, including chr6:104,819,411 (intron 12 of the Cntn6 gene) base pairs 1-1466 and 5265-7073 Mouse genome; base pairs 1467-1628 Human slow troponin I enhancer (TNNI1); base pairs 1629-1664 Spacer; base pairs 1665-4037 Human skeletal actin promoter (ACTA1); base pairs 4048-4990 Crym ORF; base pairs 5036-5264 BGH polyA; base pairs 4038-4047 and 4991-5035 Plasmid backbone. Mouse genome is indicated in lower case; TNNI1 is in lowercase italics; the spacer is in lower case underlined; ACTA1 is in lower case bold; the Crym ORF sequence is in uppercase underline; BGH polyA sequence is in uppercase; and plasmid backbone sequences are in uppercase italics. The transgene that encodes the CRYM protein is shown in FIG. 1C (SEQ ID NO:2). FIG. 1D shows the average copy number of the transgene in homozygote, heterozygote and wild type mice. This was calculated using the 2*2\hat{ }-DDCt method. Tert was used as the control gene with a known copy number of 2 and C57B16/J (BL6) mice were used as diploid controls, with two copies of the Crym gene.

FIG. 2A through FIG. 2C. FIG. 2 relates to data showing increased Crym mRNA and μcrystallin protein levels in Crym tg vs. control mice. FIG. 2A shows Crym expression in several skeletal muscles and other tissues were analyzed from 3 Crym tg and 3 control mice. FIG. 2B shows a western blot of μ-crystallin in Crym tg and control TA muscle extracts. FIG. 2C presents data for μ-Crystallin quantitation with Protein Simple's Wes instrument.

FIG. 3A through FIG. 3J present data relating to immunolabeling of μ-crystallin in control and Crym tg skeletal muscles. FIG. 3A, FIG. 3B, FIG. 3C, and FIG. 3D are cross sections and FIG. 3E, FIG. 3F, FIG. 3G, and FIG. 3H are longitudinal sections of muscle of Crym tg (FIG. 3A, FIG. 3C, FIG. 3E, FIG. 3G, and FIG. 3H) and control (FIG. 3B, FIG. 3D, and FIG. 3F) animals. FIG. 3I and FIG. 3J show isolated fibers.

FIG. 4A through FIG. 4D show contractile properties of Crym tg muscle compared to controls. FIG. 4A shows TA weight. FIG. 4B shows fiber diameter in μm. FIG. 4C shows the distribution of fiber sizes expressed as a percent of the total number of fibers. FIG. 4D shows the percent of centrally nucleated fibers (CNFs).

FIG. 5A through FIG. 5C show myosin heavy chain characteristics of Crym tg and control muscle. FIG. 5A and FIG. 5B show gastrocnemius muscle of control (FIG. 5A) and Crym tg (FIG. 5B) stained red for Type I fibers, green for Type IIa fibers, purple for Type IIb fibers, and purple for laminin to visualize the plasma membrane. FIG. 5C shows the percentage of fiber distribution in soleus, tibialis anterior (TA), and gastrocnemius (Gastroc.) in control and Crym tg muscle.

FIG. 6A, FIG. 6B, FIG. 6C, and FIG. 6D show data relating to the immunolabeling of myosin heavy chains in Crym tg and control soleus and TA cross sections.

FIG. 7A through FIG. 7H presents data concerning contractile properties of Crym tg muscle. Force frequency curves (FIG. 7A, FIG. 7B, FIG. 7D, FIG. 7E, and FIG. 7H) of gastrocnemius (FIG. 7A and FIG. 7B), soleus (FIG. 7D and FIG. 7E), and extensor digitorum longus (FIG. 7H) are shown.

FIG. 8A and FIG. 8B show data concerning the maximum velocity of Crym tg and control mice on a treadmill and the total distance run on a running wheel during the light and dark cycles for Crym tg and control mice. In FIG. 8A, a two-tailed unpaired t-test was performed (Crym tg n=7; control n=6). In FIG. 8B, kilometers (64) run during the light (day) and dark (night) cycles are shown. A two tailed unpaired t-test was performed. None of the results show statistically significant differences between Crym tg and control mice at α=0.05 (n=5).

FIG. 9A, FIG. 9B, FIG. 9C, FIG. 9D, FIG. 9E, FIG. 9F, and FIG. 9G show the maximal rate of twitch force contraction, maximal rate of twitch force relaxation, and grip strength of Crym tg and control mice. Gastrocnemius (A, D), soleus (B, E), and extensor digitorum longus (EDL) (C, F) maximal rate of twitch force contraction (+dP/dt; A-C) and maximal rate of twitch force relaxation (−dP/dt; D-F) of Crym tg and control mice. The Holm-S̆ídák test was used to determine significance (gastrocnemius and soleus n=5, EDL n=6 control n=5 Crym tg; *=p<0.05). E. Grip strength of Crym tg and control mice. Grip strength was normalized to body weight. There was not a significant difference between the grip strength of Crym tg and control mice after using a t test with Welch's correction (α=0.05; n=4).

FIG. 10A, FIG. 10B, FIG. 10C, and FIG. 10D show TA and soleus cross sections stained for fat content in Crym tg and control mice as indicated, using BODIPY 493/503.

FIG. 11 shows the average weight various tissues and the total body weight of control mice and Crym tgs. The average weight of tibialis anterior, gastrocnemius, soleus, and quadriceps muscle, subcutaneous, epididymal, mesenteric, retroperiotenal, and brown adipose fat and the total body weight of control and Crym tg mice is shown.

FIG. 12A, FIG. 12B, FIG. 12C, FIG. 12D, FIG. 12E, FIG. 12F, and FIG. 12G show characteristics, specifically count, area, perimeter, circularity, minimal Feret's diameter, roundness, and solidity, of Crym tg and control soleus myofibers immunolabeled for various myosin heavy chains. Statistical tests were used determine significance, specifically the Kruskall-Wallis Test (KW), Student's t Test (S), the Mann-Whitney U Test (MW), the Yuen-Welch t Test (YW), Welch's t Test (W), and the Brown-Forsythe Test (BF).

FIG. 13A through FIG. 13J shows data on weight gain of Crym tg and control mice on various diets. FIG. 13A (female, high fat), FIG. 13B (female, low fat), FIG. 13C (female, high simple carbohydrate), FIG. 13D (female high complex carbohydrate), FIG. 13E (female, normal), FIG. 13F (male, high fat), FIG. 13G (male, low fat), FIG. 13H (male high simple carbohydrate), FIG. 13I (male, high complex carbohydrate), FIG. 13J (male, normal).

### DETAILED DESCRIPTION

### 1. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

As used herein, the term “about” means plus or minus 20 percent of the recited value, so that, for example, “about 0.125” means 0.125±0.025, and “about 1.0” means 1.0±0.2.

As used herein, the term “subject” refers to any mammal. The terms “subject,” “individual,” “host,” and “patient,” are used interchangeably to refer to any animal, and can include simians, humans, felines, canines, equines, rodents, bovines, porcines, ovines, caprines, mammalian sport animals, and mammalian pets. A suitable subject for the invention preferably is a human that is suspected of having, has been diagnosed as having, or is at risk of developing a disease that can be ameliorated, treated or prevented by increasing CRYM in muscle. Particular preferred diseases and conditions include type 2 diabetes and obesity.

As used herein, the term “administer” and its cognates refers to introducing an agent to a subject, and can be performed using any of the various methods or delivery systems for administering agents, pharmaceutical compositions or delivering gene vectors known to those skilled in the art. Modes of administering include, but are not limited to oral administration or intravenous, subcutaneous, intramuscular or intraperitoneal injections, rectal administration by way of suppositories or enema, or local administration directly into or onto a target tissue (such as muscle), or administration by any route or method known in the art that delivers a therapeutically effective amount of the drug or composition to the cells or tissue to which it is targeted or systemically. Administration can refer to introducing the protein or, for example, a nucleic acid construct to the subject as DNA or mRNA, introducing a vector containing the nucleic acid to the subject, or introducing cells that have been transduced ex vivo with a construct, such as by electroporation or using a vector as described herein, to the subject.

As used herein, the terms “treatment,” “treating,” and the like, as used herein refer to obtaining a desired pharmacologic and/or physiologic effect. “Treatment,” includes: (a) preventing the condition or disease or symptom thereof from occurring in a subject which may be predisposed to the condition or disease but has not yet been diagnosed as having it; (b) inhibiting the condition or disease or symptom thereof, such as, arresting or slowing its development; and (c) relieving, alleviating or ameliorating the condition or disease or symptom thereof, such as, for example, causing regression of the condition or disease or symptom thereof.

As used herein, the term “pharmaceutical composition” refers to a composition that comprises an active agent, preferably μ-crystallin or a pro-drug thereof, in combination with a pharmaceutically acceptable carrier or excipient.

As used herein, the term “CRYM” refers to the protein product of the CRYM gene, which is also known as “μ-crystallin” or NADP-regulated thyroid-hormone binding protein (THBP).

### 2. Overview

A transgenic mouse that overexpressed CRYM at very high levels in muscle was produced and characterized. This mouse did have very high levels of CRYM protein in muscle, but not in other tissues. Consistent with this, muscles accumulated very high levels of T3 (thyroid hormone, triiodothyronine), with no significant changes in T4. Remarkably, serum T3 and TSH (thyroid stimulating hormone) also were unchanged. Studies of the transgenic mice in metabolic chambers indicated that they consumed the same number of calories daily as control mice, but that their energy utilization comes more from fats than from carbohydrates. RNA-seq studies showed that the muscles shift gene expression away from glycolytic metabolism and towards beta-oxidation. Consistent with this, the expression of many genes encoding contractile proteins in muscle tissue shifted from fast twitch to slow twitch or non-muscle isoforms. Thus, high levels of expression of CRYM in muscle modified energy metabolism and muscle fiber physiology.

Here we addressed the effects of high levels of Crym expression in mammalian muscle. We initially observed high levels of μ-crystallin in several muscle biopsies of patients with facioscapulohumeral muscular dystrophy (FSHD), a muscle wasting disease where patients progressively lose muscle. At the time, the cause of FSHD was unknown. Later studies showed that mRNA and protein both varied widely in expression in both healthy and diseased muscle, however, and it is now widely accepted that the protein DUX4 is the primary pathogen in FSHD. DUX4 is a transcription factor that activates CRYM expression. Thus, increases in CRYM may perturb muscle metabolism and contribute to pathology. Unusually, high levels of CRYM in skeletal muscle create conditions of mild hyperthyroidism in the muscle, including a significant shift in energy usage linked to changes in gene expression, without causing any obvious side effects normally associated with hyperthyroidism.

To examine the differences in metabolism linked to high vs. low levels of Crym expression, a transgenic mouse (Crym tg) that overexpresses μ-crystallin was produced. The Crym tg mice express the protein under the control of the human skeletal actin promoter (ACTA1) and the human slow troponin I enhancer (TNNI1) to enable skeletal muscle-specific expression. The physiologic, metabolic, and transcriptomic effects of overexpression of μ-crystallin on the function of muscle was investigated.

Significantly enriched ontological terms were seen in studies of 85 differentially expressed protein with the PANTHER Classification System. Only 7 ontological terms were significant, but of those 7 terms four involve muscle and one term, oxidation-reduction process, is metabolically related. Taken together, these terms are similar to the types of terms found via transcriptomic GO analysis.

These genes were used as a putative list of T3/T4 involved genes. Of the significant differentially expressed genes (DEGs) discovered by RNA-seq comparing Crym tg mice to controls, 211 of the 566 genes had putative T3/T4 involvement. Of those, 107 were significantly increased in expression while 104 were significantly decreased in expression (see Table 1, below, a list of genes with altered expression in Crym tg muscle, that are also altered in hypothyroid or hyperthyroid conditions). Notably, however, only a small subset of these DEGs are known to contain tetracycline-responsive elements (TREs) (see Table 2, below). This list of genes containing TREs which can act to promote or suppress the transcription of downstream genes in response to thyroid receptor complexes was cross referenced with 566 significant DEGs found via RNA-seq comparing the TA of Crym tg to the TA of control mice. padj=FDR corrected p-value. The expression of Crym is comprised of the endogenous Crym gene as well as the Crym transgene which was inserted into the Crym tg mice.

TABLE 1
List of genes with altered expression in Crym tg muscle that are also altered
in hypothyroid or hyperthyroid conditions.

| T3 Responsive Genes Significant | | | |
|---|---|---|---|
| in RNA-seq | | | |
| | Literature Review for | | Fold |
| Gene Symbol | T3/T4 Involvement | padj | Change |
| Fhl1 | | 1.77E−99 | 2.64 |
| Myoz2 | | 7.47E−25 | 1.78 |
| Lmcd1 | | 2.73E−22 | 1.62 |
| H2-Q7 | | 5.54E−19 | 1.92 |
| Hspb6 | | 1.55E−16 | 1.43 |
| Tspan12 | | 7.90E−16 | 1.64 |
| Ptpn3 | | 1.78E−12 | 1.37 |
| Egln3 | reviewed, no evidence | 5.71E−12 | 1.54 |
| Car14 | | 6.28E−12 | 1.46 |
| Pik3r1 | | 2.85E−11 | 1.41 |
| Stbd1 | reviewed, no evidence | 2.44E−10 | 1.44 |
| G0s2 | reviewed, no evidence | 4.76E−09 | 1.62 |
| Stom | | 1.05E−08 | 1.41 |
| Slc25a25 | | 1.42E−08 | 1.43 |
| H2-D1 | | 1.09E−07 | 1.24 |
| Slc38a10 | | 2.76E−07 | 1.27 |
| Adh1 | | 3.98E−07 | 1.31 |
| Emp1 | | 1.51E−06 | 1.40 |
| P2ry2 | | 1.80E−06 | 1.22 |
| Ugp2 | reviewed, evidence | 1.87E−06 | 1.22 |
| Ak4 | reviewed, no evidence | 2.73E−06 | 1.44 |
| Mdh1 | | 2.82E−06 | 1.21 |
| Irf5 | | 1.15E−05 | 1.47 |
| Vim | | 1.21E−05 | 1.24 |
| P2ry1 | | 1.33E−05 | 1.36 |
| Fmo2 | | 2.30E−05 | 1.33 |
| Acss2 | | 2.77E−05 | 1.22 |
| Got2 | | 3.37E−05 | 1.20 |
| Csrp3 | reviewed, no evidence | 4.42E−05 | 1.38 |
| Pla2g12a | reviewed, no evidence | 4.50E−05 | 1.25 |
| Dlst | | 5.42E−05 | 1.18 |
| Alpl | | 8.29E−05 | 1.29 |
| Rhot2 | | 0.00010378 | 1.19 |
| B2m | | 0.00013984 | 1.26 |
| Slc25a11 | | 0.0002713 | 1.17 |
| Vgll2 | | 0.00028075 | 1.21 |
| Abcg2 | | 0.00028772 | 1.24 |
| Alas1 | reviewed, no evidence | 0.00031762 | 1.23 |
| Slc22a4 | reviewed, no evidence | 0.00040147 | 1.36 |
| Lpl | | 0.00046686 | 1.16 |
| Lamb2 | | 0.00048446 | 1.17 |
| Rnf114 | | 0.00054973 | 1.20 |
| Anks1 | reviewed, no evidence | 0.00072273 | 1.25 |
| Aak1 | | 0.00081795 | 1.20 |
| Lrp6 | | 0.00102993 | 1.16 |
| Slc43a3 | | 0.00115456 | 1.20 |
| Ppara | | 0.00134092 | 1.27 |
| Epas1 | | 0.00138168 | 1.16 |
| Cux2 | | 0.00193831 | 1.30 |
| Car3 | | 0.00234398 | 1.16 |
| Mgll | | 0.00245892 | 1.26 |
| Ndufb5 | | 0.00262348 | 1.17 |
| Ttc19 | | 0.00270532 | 1.16 |
| Slc38a3 | | 0.00437608 | 1.16 |
| Smarca2 | reviewed, no evidence | 0.00450294 | 1.16 |
| D930015E06Rik | | 0.00549508 | 1.29 |
| Ly6c1 | | 0.00550375 | 1.16 |
| Gramd1b | | 0.00554408 | 1.27 |
| Nfkbia | | 0.0062563 | 1.25 |
| Hebp1 | | 0.00632571 | 1.31 |
| Sorbs1 | reviewed, evidence | 0.00733468 | 1.16 |
| Mapre2 | | 0.00757248 | 1.16 |
| Prdx2 | | 0.00833725 | 1.14 |
| Gpt | | 0.00978817 | 1.19 |
| Ptprk | | 0.00978817 | 1.24 |
| Sema3b | | 0.01125628 | 1.23 |
| Sdhaf4 | | 0.01197978 | 1.19 |
| Lrg1 | | 0.01361139 | 1.24 |
| Ogdh | | 0.01472432 | 1.10 |
| Pnpla2 | reviewed, no evidence | 0.01573111 | 1.11 |
| Fgf1 | | 0.01649821 | 1.27 |
| Noct | | 0.01649821 | 1.20 |
| Adprhl1 | | 0.01735839 | 1.16 |
| Pdha1 | | 0.01748964 | 1.12 |
| Fam21 | | 0.01813911 | 1.15 |
| Cobll1 | reviewed, no evidence | 0.01866365 | 1.26 |
| Sdhd | | 0.01905828 | 1.12 |
| Sdhb | | 0.01937883 | 1.12 |
| P2rx5 | | 0.02027152 | 1.20 |
| Sesn1 | | 0.02083407 | 1.18 |
| Nnt | | 0.0227335 | 1.17 |
| Fam234a | | 0.02376108 | 1.15 |
| Pdhb | reviewed, evidence | 0.0246951 | 1.11 |
| Abcb9 | reviewed, no evidence | 0.02624684 | 1.21 |
| Sirt3 | | 0.02709929 | 1.17 |
| Pecam1 | | 0.02866643 | 1.14 |
| B4galt1 | | 0.02935758 | 1.15 |
| Atp2a3 | | 0.02978623 | 1.22 |
| Acaa2 | | 0.03252215 | 1.15 |
| Lrp4 | | 0.03427458 | 1.18 |
| Mrpl47 | reviewed, evidence | 0.03457695 | 1.16 |
| Cebpb | reviewed, evidence | 0.03543368 | 1.20 |
| Sel1l3 | reviewed, no evidence | 0.03688229 | 1.14 |
| Pam | | 0.03692466 | 1.15 |
| Atad1 | | 0.03813728 | 1.11 |
| Cox7a1 | | 0.0385731 | 1.15 |
| Ccdc80 | | 0.04002404 | 1.20 |
| Adipor2 | | 0.04087305 | 1.10 |
| Rhou | | 0.04186919 | 1.25 |
| Tmem25 | | 0.04208464 | 1.18 |
| Abca9 | | 0.04220456 | 1.24 |
| Fam210a | | 0.04250672 | 1.10 |
| Lace1 | | 0.04252622 | 1.17 |
| Acta2 | | 0.04299242 | 1.18 |
| Timm44 | | 0.04414788 | 1.11 |
| Ndufa9 | | 0.04643071 | 1.12 |
| Nid1 | | 0.04918196 | 1.16 |
| Gapdh | | 0 | −6.30 |
| Vldlr | reviewed, evidence | 2.89E−17 | −1.37 |
| Ybx3 | | 8.21E−16 | −1.30 |
| Nrep | | 8.34E−16 | −1.36 |
| Itpr1 | | 5.11E−13 | −1.57 |
| Cdk19 | | 4.70E−12 | −1.37 |
| Eepd1 | | 1.09E−11 | −1.29 |
| Slc40a1 | | 1.26E−11 | −1.39 |
| Slc41a3 | | 9.40E−11 | −1.29 |
| Aldh1a1 | | 1.53E−09 | −1.32 |
| Plcd4 | | 7.82E−08 | −1.27 |
| Calm3 | | 1.42E−07 | −1.21 |
| Ociad2 | | 1.69E−07 | −1.28 |
| Nr1d1 | | 4.60E−07 | −1.25 |
| Rbfox1 | | 5.79E−07 | −1.20 |
| Sorbs2 | | 6.53E−07 | −1.32 |
| Gpr19 | | 7.45E−07 | −1.53 |
| Tmod4 | | 1.56E−06 | −1.19 |
| Plekhb1 | | 1.68E−06 | −1.27 |
| Ltbr | | 3.54E−06 | −1.33 |
| Actn3 | | 8.40E−06 | −1.22 |
| Spryd7 | | 1.20E−05 | −1.28 |
| Sar1b | | 5.47E−05 | −1.17 |
| Mospd1 | | 6.08E−05 | −1.20 |
| Cyp27a1 | reviewed, evidence | 6.15E−05 | −1.21 |
| Thns12 | | 0.00010945 | −1.38 |
| Ddx47 | | 0.00012618 | −1.24 |
| Lynx1 | | 0.00016848 | −1.17 |
| Mib1 | | 0.00018148 | −1.27 |
| Xpr1 | | 0.00021952 | −1.21 |
| Park7 | | 0.00024821 | −1.14 |
| Sqstm1 | reviewed, evidence | 0.00033996 | −1.16 |
| Deptor | | 0.00036582 | −1.22 |
| Pgm2 | reviewed, no evidence | 0.00053033 | −1.15 |
| Dbp | reviewed, evidence | 0.00063007 | −1.17 |
| Leo1 | | 0.00063577 | −1.24 |
| Msrb1 | | 0.0006502 | −1.21 |
| Myoz3 | | 0.00072273 | −1.17 |
| Setd7 | | 0.00079966 | −1.13 |
| Mgea5 | | 0.00094545 | −1.18 |
| Phkb | | 0.00101605 | −1.14 |
| Tmem106b | | 0.0012225 | −1.17 |
| Slc16a10 | reviewed, evidence | 0.00125807 | −1.18 |
| Hbp1 | | 0.00142136 | −1.18 |
| Bace1 | | 0.00151236 | −1.24 |
| Ogt | | 0.001529 | −1.14 |
| Fzd7 | | 0.00194534 | −1.23 |
| Hnrnph1 | | 0.00220436 | −1.14 |
| Anapc5 | | 0.0022629 | −1.13 |
| Polr1a | | 0.00242984 | −1.34 |
| C1s1 | | 0.00263336 | −1.27 |
| Rtn4 | | 0.00280843 | −1.14 |
| Gadd45b | | 0.00302273 | −1.24 |
| Kctd20 | | 0.00438576 | −1.16 |
| Suclg2 | | 0.00458479 | −1.16 |
| Zxdc | | 0.00505009 | −1.21 |
| Me1 | reviewed, evidence | 0.00509196 | −1.15 |
| Psme4 | | 0.00598738 | −1.12 |
| Amigo1 | | 0.00639044 | −1.17 |
| At12 | | 0.0069287 | −1.12 |
| D1Ertd622e | | 0.00748037 | −1.21 |
| Pcbp1 | | 0.00748037 | −1.13 |
| Igfbp5 | | 0.00791648 | −1.17 |
| Rcl1 | | 0.00902166 | −1.24 |
| Lgalsl | | 0.00947885 | −1.14 |
| Zfp956 | | 0.00981265 | −1.20 |
| B3galnt2 | | 0.01136638 | −1.15 |
| Tnip1 | | 0.01147469 | −1.17 |
| Fnip1 | | 0.01197978 | −1.18 |
| Zfp275 | | 0.01215467 | −1.23 |
| Akap81 | | 0.0123187 | −1.17 |
| Nup210 | | 0.01429501 | −1.15 |
| Srsf5 | | 0.01467077 | −1.14 |
| Fermt2 | | 0.01494555 | −1.11 |
| Strbp | | 0.01595775 | −1.17 |
| Lgals1 | | 0.01649821 | −1.13 |
| Ccdc91 | | 0.01653354 | −1.11 |
| Snx12 | | 0.01653354 | −1.16 |
| Gpcpd1 | reviewed, no evidence | 0.01787158 | −1.16 |
| Slc45a4 | | 0.01956123 | −1.15 |
| Gclc | reviewed, evidence | 0.02063247 | −1.19 |
| Capza2 | | 0.02079322 | −1.11 |
| Ypel3 | | 0.02277742 | −1.14 |
| Celf2 | reviewed, no evidence | 0.02480137 | −1.14 |
| Ksr1 | | 0.02866643 | −1.13 |
| Wnk1 | | 0.02957678 | −1.10 |
| Abcd2 | | 0.03060918 | −1.13 |
| Ctsc | | 0.03082624 | −1.14 |
| Calm2 | reviewed, evidence | 0.03186304 | −1.12 |
| Sccpdh | | 0.03485028 | −1.14 |
| Klhl24 | | 0.03711703 | −1.11 |
| Phc1 | | 0.03711703 | −1.17 |
| Nhs | | 0.03735671 | −1.26 |
| Adcy9 | reviewed, evidence | 0.0392952 | −1.15 |
| Cog6 | | 0.03964867 | −1.12 |
| Acadsb | | 0.04057233 | −1.14 |
| Pabpc1 | | 0.04134837 | −1.15 |
| Eif3a | | 0.04137634 | −1.10 |
| Mb21d2 | | 0.04208464 | −1.20 |
| Pigp | | 0.04267094 | −1.13 |
| Fgd4 | | 0.04278156 | −1.19 |
| Esr1 | reviewed, evidence | 0.04368916 | −1.15 |
| Tmem63a | | 0.04585533 | −1.15 |
| Ttll4 | | 0.04998611 | −1.14 |

TABLE 2
Genes with altered expression in Crym tg
that contain TREs in their promotor regions.

| Gene | padj | Fold Change |
|---|---|---|
| Crym | 0.00E+00 | 32.142 |
| Slc41a3 | 9.40E−11 | −1.287 |
| Ank2 | 7.19E−05 | 1.216 |
| Slc12a4 | 5.92E−04 | 1.277 |
| P2rx5 | 2.03E−02 | 1.202 |
| Mapk12 | 3.00E−02 | 1.109 |
| Abcd2 | 3.06E−02 | −1.133 |

### 3. Summary of Exemplary Results

A. μ-Crystallin (Crym) is expressed specifically in transgenic skeletal muscle at highly elevated levels.
B. T3 is increased ˜190 fold in the Tibialis anterior muscle of Crym tg mice.
C. Small but significant changes were seen in gene and protein expression in tg muscle towards a slow twitch, oxidative phenotype.
D. Metabolic studies show that Crym tg mice increase their use of fat as an energy source.
E. Female Crym tg mice gain weight faster on high fat or simple carbohydrate diets than controls.

### 4. Embodiments of the Invention

Because imbalances in T₃, such as hypothyroidism or hyperthyroidism, can result in a variety of maladies, thyroid hormone levels are carefully controlled under normal conditions. Regulation of thyroid hormone levels are affected by the feedback loop of thyrotropin releasing hormone (TRH), TSH, T₄, somatostatin, monocarboxylate thyroid hormone transporters (especially MCT8/10), and deiodinases (DIO1-3) that convert T₄ transported into the cell into T₃ and other metabolites. μ-Crystallin binds T₃ and its absence has been shown to increase efflux of T₃ from the cell significantly. In addition, CRYM levels can vary greatly in human muscle. Therefore we became interested in what effects μ-crystallin has in those individuals where it is highly expressed. To address this, a skeletal muscle specific transgenic mouse, Crym tg, was generated in which Crym mRNA and protein were expressed in skeletal muscle at high levels comparable to those seen in some humans. When Crym is overexpressed, metabolism shifts away from carbohydrate use toward fat use, with concomitant shifts in the expression of genes from glycolysis toward β-oxidation and from fast contractile toward slow contractile gene products.

Expression of CRYM in human skeletal muscle from 430 individuals in the GTEx database show that about 20% of individuals express CRYM at relatively high levels while the majority of people express little to no CRYM. RT-qPCR and Western blot data show a similar percentage of individuals expressing high levels of CRYM, 30-40 times higher than low expressers. Though a marked variability in CRYM expression has been observed, to the best of our knowledge no data so far correlates human CRYM expression to energy expenditure, locomotive ability, or body composition. This now has been confirmed. Crym tg mice produce approximately 94-fold more Crym mRNA as assayed by RT-qPCR and about 28 times more μ-crystallin protein in their TA muscles compared to control C57BL/6J mice. Thus, the Crym tg mouse model reproduces key features of human high expressers, producing Crym at levels that vastly outstrip control mice or human low expressers.

The enhancer from the TNNI1 gene that was used to increase expression of the Crym transgene is a slow skeletal muscle specific gene, while the promoter, taken from the human skeletal actin gene (ACTA1), shows expression in both fast and slow type fibers. It is believed that muscle-specific differences in gene expression driven by the skeletal actin promoter have not been documented, though it is regulated by a number of factors, including adrenergic signaling. Thyroid hormone itself can reduce skeletal actin expression, but only when it is introduced exogenously at levels several orders of magnitude greater than those reached in either control or Crym tg TA muscles. Studies of skeletal actin protein do not show differences in expression that depend on the muscle or fiber type composition, however, suggesting that differences in the activity of the actin promoter may not account for the results seen here. Less is known about the activity of the TNNI1 enhancer element, but, paradoxically, soleus and diaphragm, which are more slowly contracting in mice than other skeletal muscles have lower levels of Crym expression at the mRNA and protein level compared to other skeletal muscles in Crym tg mice, although they are elevated compared to controls. Thus, although unexpected, the results clearly show that the same promoter/enhancer pair can drive different levels of transgene expression in different muscle groups.

As expected from the high levels of Crym expressed in the muscles of the transgenic mice, a large accumulation of intracellular T₃ (˜190-fold) was observed in the TA muscle compared to controls. μ-Crystallin promotes accumulation of T₃ in the cytoplasm in part by sequestering the hormone. Levels of total T₃ in control mouse serum are unchanged in the transgenic mouse. At these levels, μ-crystallin in the muscle should be close to saturated with bound T₃. The same should be true for the C57BL/6J control muscle. Although the 5-fold higher enrichment in the T₃ level compared to μ-crystallin protein is not explained (the stoichiometry of binding is 1:1), μ-crystallin may promote the influx and slow the efflux of thyroid hormones by interacting directly with the T₃ transporters at the cell surface. This would be consistent with finding μ-crystallin at the sarcolemma of Crym tg muscle (see Example 3, below).

It is also not clear why serum T₄ is decreased in Crym tg mice compared to controls. TSH levels are unchanged between Crym tg and control mice, so T₄ production should be unimpaired. Lower serum T₄ may result from its increased intramuscular conversion into T₃ and its intracellular storage once it is bound to μ-crystallin in the myoplasm. Thus, the higher levels of μ-crystallin are likely to create a “sink” for T₃ within the cell that is filled by mass action of T₄ transported from the serum into the myoplasm, followed by its intracellular deiodination.

No significant differences were noted in the numbers of any fiber type or in the total number of fibers when soleus muscles of Crym tg mice were compared to controls. Similarly, no differences in fiber type were seen between control and Crym tg TA and gastrocnemius muscles. Staining for myosin heavy chains is only one metric used to quantify fiber type as slow twitch or fast twitch, however. Differences in the minimal Feret's diameter and the circularity of particular fiber types were found, among other significantly different morphological characteristics, although no physiological significance can be ascribed to these differences. Because there is a shift towards β-oxidation as determined by RER, and slow twitch fibers preferentially utilize β-oxidation compared to fast twitch fibers while also having a smaller size, soleus myofibers may be shifting towards a slower twitch phenotype. Remarkably, notwithstanding the large changes in the levels of thyroid hormones that were measured, very few other changes were seen in the structure or function of the hindlimb muscles in mice. Like the fiber types, central nucleation and fat deposition were essentially unchanged. Physiologically, overall muscle function and the function of individual muscles and muscle fibers are indistinguishable between transgenic and control samples. Thus, μ-crystallin's effects on muscle structure and function are not readily apparent, despite its massive effect on the distribution of T₃. This is consistent with the observation that most changes in gene expression that were measured in transgenic muscle are quite modest—usually less than 50% higher or lower than controls.

Because T₃ has profound effects on metabolism, the Crym tg mice were studied using metabolic cages. Uunder normal resting conditions, RER, a measure of preference for sources of metabolic energy, was significantly different between the Crym tg mice and controls. The difference in RER corresponds to a 13.7% increase in utilization of fat as an energy source over carbohydrates in Crym tg mice compared to controls.

Consistent with the changes observed in RER, GO analysis of RNA-seq data on Crym tg and controls showed an increase in expression of genes associated with β-oxidation and a decrease in genes associated with glycolysis. As the murine TA muscle is almost entirely fast twitch fibers, which are primarily glycolytic, the 566 significantly differentially expressed genes were compared to a list of 1343 fast and slow fiber type genes generated by Drexler et al. Onew hundred six (about 19%) of the genes altered in expression in the Crym tg mice encode contractile or other proteins related to fiber type, and that the expression of genes associated with fast fiber types significantly decreased while that of genes associated with slow fiber types significantly increased (see Example 9, below).

In contrast, proteomic studies results revealed 26 significantly differentially expressed, fiber type-specific proteins, of which 7 shifted towards a slower phenotype while 19 shifted towards a faster phenotype (see Table 3, below). As reported in the art, high levels of TH have little effect on fast twitch muscles but cause slow twitch muscles to exhibit faster twitch characteristics. Because mice have very few slow twitch fibers and the Crym tg mouse is subject to far less than the thyrotoxic levels of TH used in previous rodent studies, it is not surprising that minimal effects of Crym overexpression were seen. However, the data show for the first time that high levels of T₃ in myocytes may cause curious transcriptional/translational shifts in which slow fiber type genes increase in expression while fast fiber type proteins increase in expression.

TABLE 3
Significant biological GO terms derived from a list of 85 differently
expressed proteins via LC MS/MS proteomics comparing Crym tg
mice to controls.

| Term | Count | P value | Fold Enrichment |
|---|---|---|---|
| Oxidation-reduction | 11 | 0.0004 | 3.9761 |
| process | | | |
| Cardiac muscle | 4 | 0.0010 | 20.3626 |
| contraction | | | |
| Cardiac myofibril | 3 | 0.0010 | 61.0878 |
| assembly | | | |
| Detection of muscle | 2 | 0.0161 | 122.1757 |
| stretch | | | |
| Protein refolding | 2 | 0.0358 | 54.3003 |
| Cardiac muscle | 2 | 0.0358 | 54.3003 |
| hypertrophy | | | |
| Regulation of mitotic | 2 | 0.0474 | 40.7252 |
| spindle assembly | | | |

Coupled with the shift towards a preference for fat utilization and increased expression of genes involved in β-oxidative metabolism, 11 proteins related to the metabolic oxidation-reduction process, and decreased expression of genes involved in carbohydrate metabolism, indicates that the accumulation of T₃ in muscle is associated with a shift towards a slower twitch, more β-oxidative phenotype.

Earlier studies used thyrotoxic doses of T₃ and T₄ in rats to alter skeletal muscles. These doses induced a shift in slow twitch soleus muscle from slow to faster characteristics, but without accompanying changes in contractile velocity. They had no effect on fast twitch extensor digitorum longus (EDL) muscles (23). Consistent with the observations here, these results suggest that the contractile properties of muscle may not always correspond to its histochemical and biochemical properties, at least when thyroid hormones are elevated. The levels reached in Crym tg muscle are considerably below thyrotoxic levels, however. A later study examined the properties of muscle in mice lacking thyroid hormone receptors and, again found changes in soleus but not EDL. In this case, however, soleus muscles in the knockouts showed even slower twitch characteristics than in controls, consistent with changes seen in hypothyroid mice. These results suggest that any elevation in T₃ related to transgenic expression of Crym is more likely to appear in slow twitch than in fast twitch muscles. Given the fact that only about 60% of murine soleus muscles are slow and the effects of Crym overexpression are subtler than either thyrotoxicosis or the complete ablation of the thyroid hormone receptor, significant differences in the physiological properties of Crym tg muscles might not be expected. This is congruent with the observation that the changes in gene expression of the myosin heavy and light chains in TA muscle are relatively small and inconsistent (see Table 4, below). Myosin heavy and light chain, mRNA and protein expression was measured with RNA-seq and LC.MS/MS respectively. In Table 4, * indicates when the transcript or protein was detected, followed by the p value and fold change in expression in Crym tg TA muscles vs controls. While no changes were seen in fiber type as measured by myosin heavy chain presence in soleus muscle, smaller Type I/IIb and Ha fibers were consistent with a shift towards a slower muscle twitch phenotype.

TABLE 4
Myosin Heavy and Light Chain, mRNA and Protein Expression

| | | | RNA-seq | | | LC.MS/MS | | |
|---|---|---|---|---|---|---|---|---|
| | | Heavy/Light | Transcript | p | Fold | Protein | p | Abundance |
| Gene | Function | Chain | Measured | Value | Change | Measured | Value | Ratio |
| Myh2 | Type IIa | heavy | * | 1E−56 | 1.99 | * | | |
| Myl2 | slow | light | * | | | * | 5E−16 | 2.67 |
| | skeletal/cardiac | | | | | | | |
| Myh1 | Type IIx | heavy | * | 7E−14 | 1.44 | * | | |
| Myh4 | Type IIb | heavy | * | 3E−05 | −1.25 | * | | |
| Myl12a | smooth/non-muscle | light | * | 4E−05 | 1.19 | | | |
| Myl3 | slow | light | * | 0.0012 | 1.36 | * | | |
| | skeletal/cardiac | | | | | | | |
| Myh10 | non-muscle | heavy | * | 0.0014 | 1.23 | | | |
| Myh11 | smooth muscle | heavy | * | 0.0049 | 1.22 | * | | |
| Myh7b/14 | slow muscle/non- | heavy | * | | | * | 0.0057 | 1.38 |
| | muscle | | | | | | | |
| Myh7 | Type I | heavy | * | | | * | | |
| Myh8 | neonatal/perinatal | heavy | * | | | * | | |
| Myh6 | cardiac | heavy | * | | | * | | |
| Myh13 | superfast | heavy | * | | | * | | |
| Myh9 | non-muscle | heavy | * | | | * | | |
| Myh3 | embryonic | heavy | * | | | * | | |
| Myh15 | slow skeletal/non- | heavy | | | | * | | |
| | muscle | | | | | | | |
| Myl9 | smooth/non- | light | * | | | * | | |
| | muscle | | | | | | | |
| Myl4 | embryonic/cardiac | light | * | | | * | | |
| Myl1 | fast skeletal | light | * | | | * | | |
| Myl10 | non-muscle | light | | | | | | |
| Myl6 | smooth/non- | light | * | | | * | | |
| | muscle | | | | | | | |
| Myl12b | non-muscle | light | * | | | * | | |
| Mylpf | fast skeletal | light | * | | | * | | |
| Myl6b | slow skeletal/non- | light | * | | | * | | |
| | muscle | | | | | | | |

Significantly greater rates of increase in body weight of female Crym tg compared to control mice on high fat or high simple carbohydrate diets. There may be a sexually dimorphic effect that higher levels of μ-crystallin has on metabolism because a similar significant increase in body weight of Crym tg was not observed in males on these diets. Indeed Chen et al. found that mice with two X chromosomes had “accelerated weight gain on a high fat diet” and up to a 2-fold increase in adiposity compared to XY mice, showing a clear sexual dimorphism for fat storage. Thus, μ-crystallin may affect metabolism in a sexually dimorphic way. See Example 8, below.

The changes discussed here are novel. The only comprehensive studies of thyroid hormone-dependent gene expression in mice to date were performed in liver tissue, though an older study has been reported on skeletal muscle in men. There are 5,129 significantly differentially expressed genes shared across three whole genome microarrays comparing hyperthyroid mouse liver to euthyroid or hypothyroid mouse liver (GEO DataSets ID: 200068803, 200065947, 200021307). Of the 566 significant DEGs identified by RNA-seq, 211 showed changes in these studies of liver. 107 were significantly increased in expression while 104 were significantly decreased in expression (see Table 1, above). Notably, however, only a small subset of these DEGs are known to contain TREs (see Table 2, above). Despite the fact that T₃ is highly elevated in the TA muscles of Crym tg mice, the changes in mRNA and protein levels are modest. This is in keeping with earlier studies of the relatively modest effects of more extreme changes in thyroid hormone signaling in rodent muscle.

The shift in gene expression and metabolism in Crym tg mice also can be relevant to human physiology and perhaps to the pathophysiology of facioscapulohumeral muscular dystrophy (FSHD). CRYM levels in human muscles vary widely, but no distinctive physiological differences have been linked to this variability. Such differences can appear in muscle stressed by disease, however. In particular, CRYM is likely to be linked directly or indirectly to FSHD. DUX4 expressed in FSHD muscle increases CRYM expression, and μcrystallin in mice promotes a shift in RER and gene expression consistent with a shift toward a slower fiber type, which if pronounced enough would generate less force upon contraction. Notably, force generated by fast twitch muscle fibers is significantly reduced in FSHD. Although Crym tg mice do not show a decrease in force, the changes documented in TA muscle could be associated with such a decrease if it manifests over decades, the time course of the disease in man.

In summary, the high levels of μ-crystallin in skeletal muscle greatly increases T₃ levels in muscle, shifts metabolism to favor the use of fat over carbohydrates as an energy source, and enhances the expression of genes typical of slow skeletal muscle. Remarkably, the morphological and physiological characteristics of the muscle are not significantly altered. Therefore the information presented here suggests that the higher levels of μ-crystallin seen in some humans regulate gene expression and metabolism in similar, subtle ways. Individuals showing high levels of μ-crystallin expression in muscle likely will be more resistant to diabetes and obesity, and that mechanisms that up-regulate μ-crystallin will be beneficial in treating these conditions.

The invention is useful for mammalian subjects, including rats, mice, dogs, cats, primates, or any mammal in need of treatment for diabetes or obesity, preferably humans. Such a subject in need of treatment includes any mammal that has or is susceptible to a disease or condition that can be ameliorated by shifting metabolism to favor the use of fats over carbohydrates as an energy source or increased beta oxidation. Preferably, the disease or condition is a lipolytic disorder, a lipogenic disorder, a glycolytic disorder, a gluconeogenic disorder, type 2 diabetes, or obesity and more preferably is type 2 diabetes, obesity or both.

The compounds of the invention include CRYM protein or viral vectors, such as AAV or baculovirus, containing a CRYM transgene, chemical compounds (see Table 5 for a list of chemicals currently known to increase CRYM expression), protein agonists such as transcription factors or targeted overexpression systems. Some method embodiments of the invention involve direct injection with CRYM protein or RNA, transgenesis, addition of CRYM genes through genome editing, addition of an alternate CRYM promoter through genome editing, transfection of stable or transient CRYM overexpression plasmids or artificial chromosomes, genome editing of SNPs that may control CRYM expression, addition of CRYM stabilizing protein complexes or chemicals, addition of stabilizing DNA or RNA sequences provided transiently or produced intracellularly.

TABLE 5
Chemical Compounds that Increase CRYM Expression

| Chemical Name | Interaction |
|---|---|
| pirinixic acid | [pirinixic acid binds to and results in increased activity of |
| | PPARA protein] which results in increased expression of |
| | CRYM mRNA |
| 4-(5-benzo(1,3)dioxol-5-yl- | [NOG protein co-treated with entinostat co-treated with |
| 4-pyridin-2-yl-1H-imidazol- | dorsomorphin co-treated with 4-(5-benzo(1,3)dioxol-5-yl-4- |
| 2-yl)benzamide | pyridin-2-yl-1H-imidazol-2-yl)benzamide] results in increased |
| | expression of CRYM mRNA |
| Choline | [Methionine deficiency co-treated with Choline deficiency co- |
| | treated with Folic Acid deficiency] results in increased |
| | expression of CRYM mRNA |
| dorsomorphin | [NOG protein co-treated with entinostat co-treated with |
| | dorsomorphin co-treated with 4-(5-benzo(1,3)dioxol-5-yl-4- |
| | pyridin-2-yl-1H-imidazol-2-yl)benzamide] results in increased |
| | expression of CRYM mRNA |
| entinostat | [NOG protein co-treated with entinostat co-treated with |
| | dorsomorphin co-treated with 4-(5-benzo(1,3)dioxol-5-yl-4- |
| | pyridin-2-yl-1H-imidazol-2-yl)benzamide] results in increased |
| | expression of CRYM mRNA |
| Estradiol | [estradiol 3-benzoate co-treated with [Testosterone co-treated |
| | with Estradiol]] results in increased expression of CRYM |
| | mRNA |
| estradiol 3-benzoate | [estradiol 3-benzoate co-treated with [Testosterone co-treated |
| | with Estradiol]] results in increased expression of CRYM |
| | mRNA |
| Folic Acid | [Methionine deficiency co-treated with Choline deficiency co- |
| | treated with Folic Acid deficiency] results in increased |
| | expression of CRYM mRNA |
| Methionine | [Methionine deficiency co-treated with Choline deficiency co- |
| | treated with Folic Acid deficiency] results in increased |
| | expression of CRYM mRNA |
| Oxaliplatin | [Oxaliplatin co-treated with Topotecan] results in increased |
| | expression of CRYM mRNA |
| Testosterone | [estradiol 3-benzoate co-treated with [Testosterone co-treated |
| | with Estradiol]] results in increased expression of CRYM |
| | mRNA |
| Topotecan | [Oxaliplatin co-treated with Topotecan] results in increased |
| | expression of CRYM mRNA |
| Phenobarbital | NR1I3 protein affects the reaction [Phenobarbital results in |
| | increased expression of CRYM mRNA] |
| 1,2,5,6-dibenzanthracene | 1,2,5,6-dibenzanthracene results in increased expression of |
| | CRYM mRNA |
| 1,3-butadiene | 1,3-butadiene results in increased expression of CRYM |
| | mRNA |
| 1,4-bis(2-(3,5- | 1,4-bis(2-(3,5-dichloropyridyloxy))benzene results in |
| dichloropyridyloxy))benzene | increased expression of CRYM mRNA |
| abrine | abrine results in increased expression of CRYM mRNA |
| Aldehydes | Aldehydes results in increased expression of CRYM mRNA |
| Amphetamine | Amphetamine results in increased expression of CRYM |
| | mRNA |
| archazolid B | archazolid B results in increased expression of CRYM mRNA |
| Asbestos, Crocidolite | Asbestos, Crocidolite results in increased expression of |
| | CRYM mRNA |
| Benzo(a)pyrene | Benzo(a)pyrene results in increased expression of CRYM |
| | mRNA |
| BEP protocol | BEP protocol results in increased expression of CRYM |
| | mRNA |
| bis(4-hydroxyphenyl)sulfone | bis(4-hydroxyphenyl)sulfone results in increased expression |
| | of CRYM mRNA |
| bisphenol F | bisphenol F results in increased expression of CRYM mRNA |
| butyraldehyde | butyraldehyde results in increased expression of CRYM |
| | mRNA |
| Carbon Tetrachloride | Carbon Tetrachloride results in increased expression of |
| | CRYM mRNA |
| Cyclosporine | Cyclosporine results in increased expression of CRYM |
| | mRNA |
| Dibutyl Phthalate | Dibutyl Phthalate results in increased expression of CRYM |
| | mRNA |
| Dietary Fats | Dietary Fats results in increased expression of CRYM mRNA |
| Dihydrotestosterone | Dihydrotestosterone results in increased expression of CRYM |
| | mRNA |
| entinostat | entinostat results in increased expression of CRYM mRNA |
| Ethanol | Ethanol results in increased expression of CRYM mRNA |
| Fenretinide | Fenretinide results in increased expression of CRYM mRNA |
| Methamphetamine | Methamphetamine results in increased expression of CRYM |
| | protein |
| Methyl Methanesulfonate | Methyl Methanesulfonate results in increased expression of |
| | CRYM mRNA |
| ochratoxin A | ochratoxin A results in increased expression of CRYM protein |
| Oxaliplatin | Oxaliplatin results in increased expression of CRYM mRNA |
| Palm Oil | Palm Oil results in increased expression of CRYM mRNA |
| pentanal | pentanal results in increased expression of CRYM mRNA |
| Phenobarbital | Phenobarbital results in increased expression of CRYM |
| | mRNA |
| Potassium Dichromate | Potassium Dichromate results in increased expression of |
| | CRYM mRNA |
| propionaldehyde | propionaldehyde results in increased expression of CRYM |
| | mRNA |
| Propylthiouracil | Propylthiouracil results in increased expression of CRYM |
| | mRNA |
| Quercetin | Quercetin results in increased expression of CRYM mRNA |
| Sesame Oil | Sesame Oil results in increased expression of CRYM mRNA |
| Silicon Dioxide | Silicon Dioxide results in increased expression of CRYM |
| | mRNA |
| Sunitinib | Sunitinib results in increased expression of CRYM mRNA |
| Tetrachlorodibenzodioxin | Tetrachlorodibenzodioxin results in increased expression of |
| | CRYM mRNA |
| Topotecan | Topotecan results in increased expression of CRYM mRNA |
| Tretinoin | Tretinoin results in increased expression of CRYM mRNA |
| Valproic Acid | Valproic Acid results in increased expression of CRYM |
| | mRNA |

Preferred compounds in Table 5, for use with the inventive methods for treatment of obesity and type 2 diabetes include: pentanal, tretinoin, fenretinide, estradiol 3-benzoate, and dihydrotestosterone.

The compounds listed above can be administered as a base compound, and any pharmaceutically acceptable hydrate, solvate, acid or salt, and can be amorphous or in any crystalline form, or as an oil or wax. Any pharmaceutically acceptable salt can be used, as may be convenient. Generally, these salts are derived from pharmaceutically and biologically acceptable inorganic or organic acids and bases or metals. Examples of such salts include, but are not limited to: acetate, adipate, alginate, ammonium, aspartate, benzoate, benzenesulfonate (besylate), bicarbonate, bisulfate, butyrate, citrate, camphorate, camphorsulfonate, carbonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, magnesium, maleate, malonate, methanesulfonate (mesylate), 2-naphthalenesulfonate, nicotinate, nitrate, oxalate, palmoate, pectinate, persulfate, 3-phenylpropionate, phosphate, picrate, pivalate, potassium, propionate, salicylate, sodium, succinate, sulfate, tartrate, thiocyanate, toluenesulfonate (tosylate) and undecanoate salts.

In certain preferred embodiments, the compounds described herein are formulated and are administered as a pharmaceutical composition that includes a pharmaceutically acceptable carrier and one or more pharmaceutical agent, including one or more of the compounds described herein, and including one or more of the inventive compounds described herein with an additional agent, such as drug of another class.

A pharmaceutically acceptable carrier refers to any convenient compound or group of compounds that is not toxic and that does not destroy or significantly diminish the pharmacological activity of the therapeutic agent with which it is formulated. Such pharmaceutically acceptable carriers or vehicles encompass any of the standard pharmaceutically accepted solid, liquid, or gaseous carriers known in the art. A suitable carrier depends on the route of administration contemplated for the pharmaceutical composition.

Routes of administration are determined by the person of skill according to convenience, the health and condition of the subject to be treated, and the location and stage of the condition to be treated. Such routes can be any route which the practitioner deems to be most effective or convenient using considerations such as the patient, the patient's general condition, and the specific condition to be treated, including local or systemic administration. For example, routes of administration can include, but are not limited to local or parenteral routes, including: oral, intravenous, intraarterial, intrathecal, intramuscular, subcutaneous, intradermal, intraperitoneal, rectal, vaginal, topical, nasal, local injection, buccal, transdermal, sublingual, inhalation, transmucosal, wound covering, direct injection into an area to be treated, and the like. The administration can be given by transfusion or infusion, and can be administered by an implant, an implanted pump, or an external pump, or any device known in the art.

Therefore, the forms which the pharmaceutical composition can take will include, but are not limited to: tablets, capsules, caplets, lozenges, dragees, pills, granules, oral solutions, powders or granules for dilution, powders for inhalation, vapors, gases, sterile solutions or other liquids for injection or infusion, transdermal patches, buccal patches, inserts and implants, rectal suppositories, vaginal suppositories, creams, lotions, oils, ointments, topical coverings (e.g., wound coverings and bandages), suspensions, emulsions, lipid vesicles, and the like.

Treatment regimens of the chemical compounds contemplated for use with the invention include a single administration or a course of administrations lasting two or more days, including a week, two weeks, several weeks, a month, two months, several months, a year, or more, including administration for the remainder of the subject's life. The regimen can include multiple doses per day, one dose per day or per week, for example, or a long infusion administration lasting for an hour, multiple hours, a full day, or longer.

Dosage amounts per administration of these compounds include any amount determined by the practitioner and will depend on the size of the subject to be treated, the state of the health of the subject, the route of administration, the condition to be treated, the severity of the condition, and the like. In general, it is contemplated that for the majority of subjects, a dose in the range of about 0.01 mg/kg to about 100 mg/kg is suitable, preferably about 0.1 mg/kg to about 50 mg/kg, more preferably about 0.1 mg/kg to about 10 mg/kg, and most preferably about 0.2 mg/kg to about 5 mg/kg are useful. This dose can be administered weekly, daily, or multiple times per day. A dose of about 0.1 mg, 0.2 mg, 0.25 mg, 0.5 mg, 1 mg, 5 mg, 10 mg, 20 mg, 40 mg, 80 mg, 100 mg, 250 mg, 500 mg, or 1000 mg can be administered.

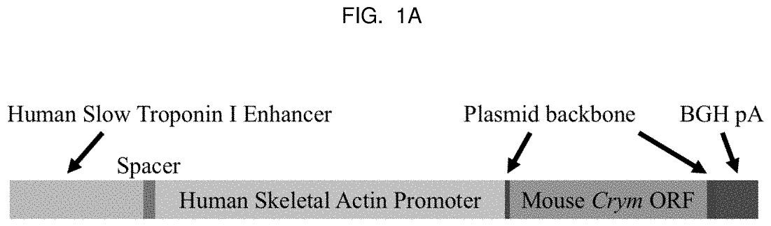

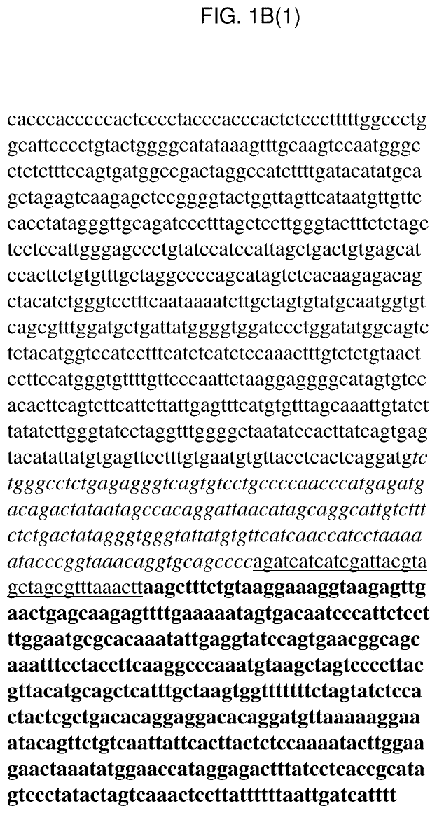

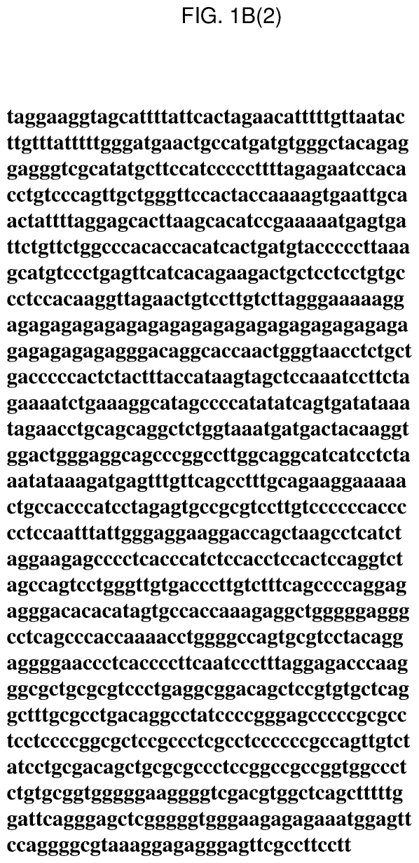

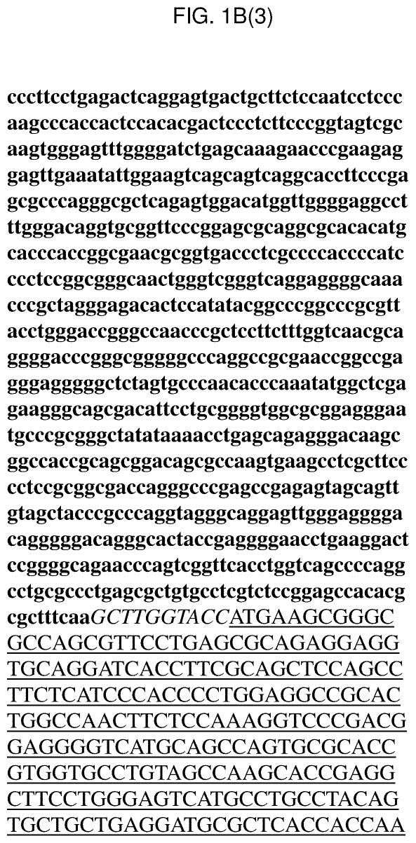

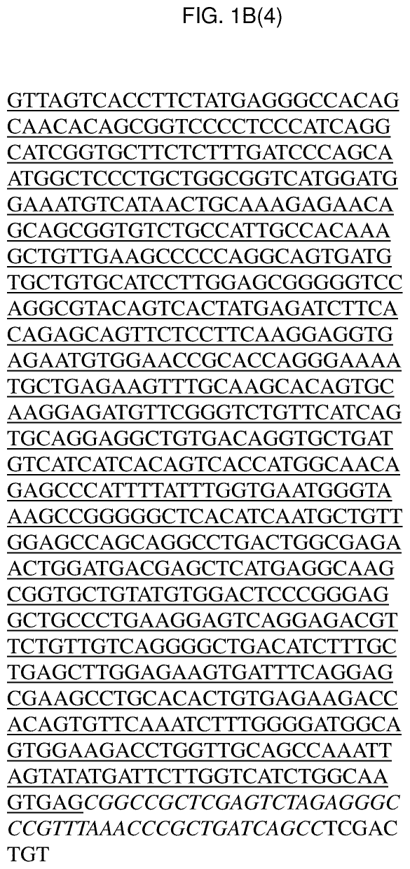

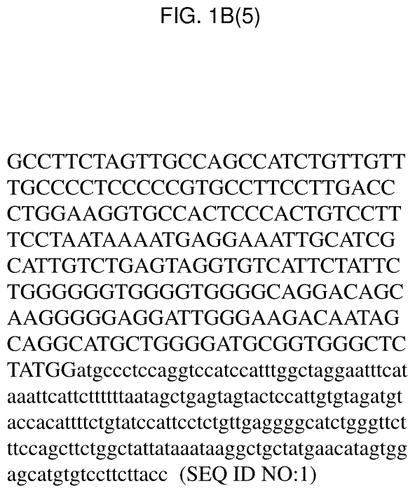

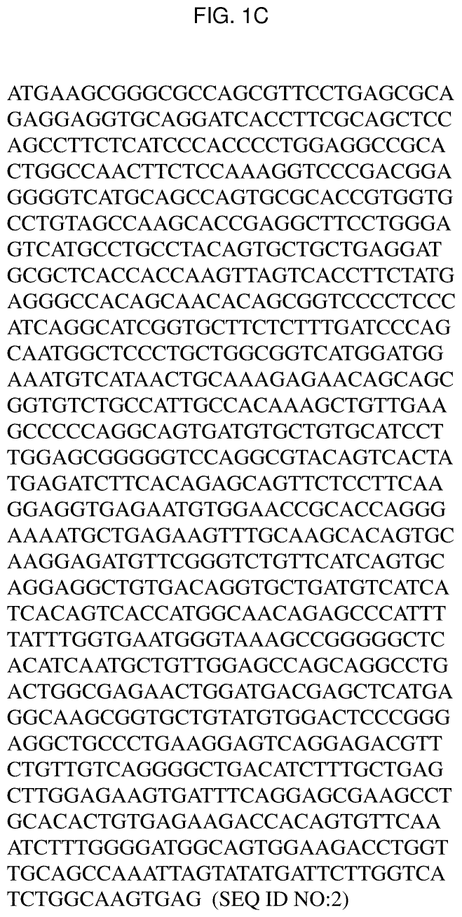

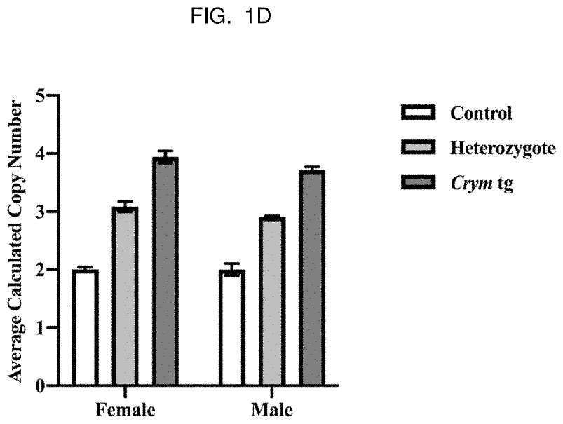

In other embodiments of the invention, subjects in need can be treated with a transgene, preferably inserted in to an AAV vector for administration to a subject. Such techniques are well known to those of skill in the art. The methods of preparing a suitable vector and determining doses for a particular patient is within the skill in the art, but dosages of vector (for example, AAV) will range from about 3×10⁹ vg/kg (vector genomes/kilogram) to about 3×10¹⁴ vg/kg and may be delivered intravenously, intramuscularly, subcutaneously, or retroperitoneally. Preferably the vector contains a transgene that encodes CRYM protein, for example SEQ ID NO:2. See FIG. 1B and FIG. 1C.

Thus, the invention comprises methods of treatment for patients and methods of modulating CRYM expression or content in the body. AAV or small molecules would be delivered to patients at appropriate doses and at a given dosing regimen. Subject inclusion criteria would include individuals with lipolytic disorders, lipogenic disorders, glycolytic disorders, gluconeogenic disorders, type 2 diabetes, obesity, or any combination of the aforementioned disorders. Individuals would not have other major underlying health disorders and may range in age from 18 to 65 years of age. The dose of AAV or of a small molecule may be given one to three times and may be separated by one week up to two month in between doses.

The studies presented here demonstrate that high levels of Crym, expressed specifically in skeletal muscles, result in an increase in T₃ levels in muscle of ˜200-fold. This is accompanied by a change in metabolism from glycolytic towards oxidative pathways, and by small but significant changes in gene and protein expression that correspond with shifts from glycolytic to oxidative and fast to slow twitch phenotypes. Notably, there were no significant changes in muscle structure or function in the whole animal, isolated muscles, or individual myofibers. This study therefore provides further understanding of the roles of Crym and thyroid hormone in regulating metabolism and gene expression in skeletal muscle and provides methods for treatment of diseases and conditions which would benefit from increased CRYM protein function, such as obesity and type 2 diabetes. 4. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: Materials and Methods

### A. Materials

Unless otherwise stated, all biologics were from Sigma-Aldrich™ and all salts were from Thermo Fisher™

### B. Creation of the Crym tg Mouse

Mouse Crym was cloned downstream of the human skeletal actin promoter (ACTA1) and human slow troponin I enhancer element (TNNI1), (kindly provided by Dr. J. Molkentin, Cincinnati Children's Hospital Medical Center), to limit expression to differentiated skeletal muscle. The expression construct was linearized and injected into C57BL/6J mouse embryos at the Genome Modification Facility, Harvard University (Cambridge, Mass.). See FIG. 1. Mice received from the Harvard facility were genotyped by PCR and then rederived by artificial insemination of C57BL/6J mice, due to pinworm infestation. This was performed by Veterinary Resources, University of Maryland School of Medicine. The rederived offspring were bred; all mice were tail snipped at weaning. Genomic DNA was purified from mouse tail snips using the Nucleon Genomic DNA extraction kit (Tepnel Life Sciences™, Scotland). PCR of genomic DNA was used to identify mice positive for the transgene, with the following primers:

forward:
(SEQ ID NO: 3)
TGGCCACGCGTCGACTAGTACG
reverse:
(SEQ ID NO: 4)
AATTCGTACTAGTCGACGCGTGGCC.

As it was initially difficult to differentiate between heterozygotes and homozygotes with PCR methods, qPCR was done on several of the Crym-positive genomic DNA samples. Mice with higher levels of Crym were then bred to controls and the F1 and F2 offspring were crossed and screened to generate probable homozygotes. Mice were identified as tg/tg homozygotes if after crossing with controls they gave at least 12 Crym-positive offspring and no Crym-negative offspring by PCR. Homozygotes were then bred together to establish the line of Crym tg/tg mice.

All histologic and physiologic experiments used approximately three-month-old, male control and Crym tg mice that were anesthetized under isoflurane (2.5%). Euthanasia was by cervical dislocation under anesthesia. All procedures were approved by the Institutional Animal Care and Use Committee, University of Maryland School of Medicine.

### C. Back-Crossing and Genotyping

Crym tg mice were backcrossed with control C57BL/6J mice. Non-littermate F1 heterozygotes were then bred together to generate F2 mice. Tail snips of Crym tg, C57BL/6J, F1 heterozygotes, and F2 mice were then taken at 6 weeks of age or older. Genomic DNA was extracted from the tail snips by following the manufacturers protocol from the PureLink™ Genomic DNA Mini Kit (K182001; Invitrogen™) modified only by substituting DirectPCR Lysis Reagent™ (Tail) (102-T; Viagen Biotech™, Los Angeles, Calif.) in place of PureLink™ Genomic Digestion Buffer. Crym tg, F1 heterozygotes, and C57BL/6J mice were genotyped using a multiplexed probe-based assay from IDT for Crym and Tert. The Crym assay was designed such that it would produce the same amplicon from the native Crym gene as well as the inserted transgene. A synthetic Crym amplicon was used as an interplate control and Tert was used as the control for copy number. RT-qPCR was used to determine copy number and average normalized C_{q} for each mouse. The amplification protocol followed the manufacturer's instructions (IDT). Mice were genotyped to one of the three genotypes when the calculated copy number and average normalized C_{q} was closest to a known genotype (i.e. Crym tg, C57BL/6J, or F1 heterozygotes).

### D. Sequencing the Transgene Insertion Site

Genomic DNA obtained from Crym tg mice was digested with EcoRI. We designed and had synthesized a double stranded short oligonucleotide with overhanging sequences corresponding to an EcoRI digestion that we called Crym-Adaptor. Crym-Adaptor was ligated to the digested genomic DNA. The resulting ligation fragments were amplified using a forward primer in the multiple cloning site of the transgene and a reverse primer in the Crym-Adaptor sequence. Nested primers were used to specifically amplify the sequence. The products of this last reaction were purified by gel purification and sequenced from both ends. The sequence was then evaluated with NCBI BLASTn.

### E. Staining of Longitudinal and Cross Sections for μ-Crystallin

Mice were perfusion-fixed with 2% paraformaldehyde in phosphate-buffered saline (PBS). TA and soleus muscles were collected, snap frozen in a liquid nitrogen slush, mounted in Optimal Cutting Temperature (OCT) (Fisher Healthcare™, Hampton, N.H.) and sectioned at 10-20 μm with a Reichert Jung™ cryostat (Leica™, Buffalo Grove, Ill.). Sections were stained using the Mouse-On-Mouse (M.O.M.) Basic Kit (BMK-2202; Vector™ Labs, Burlingame, Calif.). Sections were incubated for at least 1 hour at room temperature in Mouse-on-Mouse (MOM) blocking reagent followed by a 10 minutes of incubation in MOM diluent. Sections were incubated overnight at 4° C. in mouse monoclonal anti-μ-crystallin antibody (GTX84654; GeneTex™ Inc., Irvine, Calif.) diluted 1:100. Some sections were also incubated with rabbit anti-desmin (PA5-16705; Thermo Fisher™, Waltham, Mass.), also diluted 1:100. Sections were washed in PBS and then stained for 1 hour at room temperature with Alexa Fluor™ 568 goat anti-mouse secondary antibody (A11031) and Alexa Fluor™ 488 goat anti-rabbit (A32731), both from Alexa™ Molecular Probes (Invitrogen™, Carlsbad, Calif.), diluted 1:200. Ali antibodies were made up in MOM diluent. Samples were washed with PBS, mounted in Vectashield+DAPI (H-1500; Vector™ Laboratory), and imaged with a Nikon™ W-1 spinning disc confocal microscope (Nikon™ USA, Melville. N.Y.). We used identical laser power and exposure settings to compare cross sections of TA muscle, of soleus muscle, and of longitudinal sections of TA muscle, but we adjusted them for each set of comparisons to optimize image clarity.

### F. Fiber Type Staining

We used a slightly modified version of Kammoun et al.'s fiber typing protocol as known in the art. Primary murine monoclonal antibodies specific for the myosin isoforms Myh7 (type I; BA-D5, isotype IgG2b), Myh2 (type IIa; SC-71, isotype IgG1), and Myh4 (type IIb; BF-F3, isotype IgM) were from the Developmental Studies Hybridoma Bank, (Iowa City, Iowa). Alexa Fluor™ 647 conjugated wheat germ agglutinin (WGA) was used to stain the myofiber surface (W32466, Thermo Fisher™). The 4 reagents were used on 10 μm thick cross sections of snap frozen soleus and TA muscles (n=5). The following secondary antibodies were used: goat anti-mouse IgG2b Cross-Adsorbed Secondary Antibody Alexa Fluor™ 568 (A-21144, Thermo Fisher™), goat anti-mouse IgG1 Cross-Adsorbed Secondary Antibody Alexa Fluor™ 488 (A-21121, Thermo Fisher), and goat anti-mouse IgM mu chain Alexa Fluor™ 405 (ab175662, Abcam). Slides were imaged on a Nikon™ W-1 spinning disc microscope. Laser power and exposure were identical for comparisons of sections of control and tg TA muscles, and for soleus muscles, but were different between these tissues. We used 5 Crym tg and 4 control mice to study soleus fiber type. Four stacked images were taken at approximately 2 μm intervals and stacked to generate a single image, which were flattened using Aguet et al.'s Model-Based 2.5-D Deconvolution for Extended Depth of Field algorithm. We determined that an η₀=0.2 and a η₁=1.3 were optimal in our images to generate an in-focus image of the flattened z stacks. For soleus muscle sections we used Myosoft™, a Fiji™ macro, to categorize fibers by their myosin heavy chain composition and measure several traits (count, area, perimeter, circularity, minimal Feret's diameter, roundness, and solidity). Statistical analysis used Real Statistics Resource Pack software (Release 6.8) as a plugin in Microsoft Excel™ with a=0.05. Normality of the 7 measured metrics for each fiber type (I, IIa, IIb, IIx, I/IIa, I/IIb, IIa/IIb, and I/IIa/IIb) were determined with the Shapiro-Wilks and d'Agostino-Pearson tests, while scedasticity was determined with the mean, median, and trimmed method in Levene's test. If a dataset failed any individual test for normality or homoscedasticity, it was considered not normal and heteroscedastic. Measurements that were both normal and homoscedastic were tested for significance using Student's t test, while measurements that were normal but heteroscedastic were tested for significance using either Welch's t test or the Brown-Forsythe test. The Kruskal-Wallis test for significance was used on measurements that failed the tests for normality but were homoscedastic. Either the Yuen-Welch or the Mann-Whitney U test were used on measurements that failed tests for both normality and homoscedasticity.

### G. Fat Staining

Cryosections of 5 snap frozen soleus and TA muscles from control and Crym tg mice were stained with BODIPY (493/503) according to Spangenburg et al. and imaged with a Nikon™ spinning disk microscope (see above). Laser power and exposure settings were maintained for each section regardless of mouse strain, but these were altered between TA and soleus tissues. Accordingly, look up table values were also different from TA to soleus samples but the same within a tissue regardless of mouse strain.

### H. μ-Crystallin Staining of Isolated FDB Fibers

FDB muscles were harvested bilaterally and digested in Dulbecco's modified Eagle's medium with 4 mg/mL type II collagenase (Gibco™, Thermo Fisher™ Scientific, Waltham, Mass.) for 3 h at 37° C. Tissue was transferred to FDB medium (Dulbecco's modified Eagle's medium with 2% BSA, 1 μl/mL gentamicin, and μl/ml fungizone). Single myofibers were mechanically separated by trituration and allowed to incubate overnight. Isolated fibers were plated down on coverslips coated with Geltrex™ (A1413201; Thermo Fisher™ Scientific) for 2 hours. Coverslips were fixed in 2% paraformaldehyde at room temperature for 15 minutes. They were then permeabilized with 0.25% Triton™ X-100 in PBS for 10 minutes and stained with antibody to μ-crystallin (H00001428-M03; Abnova, Taiwan), diluted 1:100, followed by Alexa™ Fluor 488, goat anti-mouse secondary antibody (A11029; Alexa™ Molecular Probes, Invitrogen™), diluted 1:200, with the MOM kit, as described above. Each incubation was for 1 hour at room temperature. Samples were imaged on a Zeiss™ 510 Duo microscope (Carl Zeiss™, Thornwood, N.Y.).

### I. CNFs and Minimal Feret's Diameter of TA Cross Sections

Cross sections of TA muscles were used to measure centrally nucleated fibers (CNFs) and minimal Feret's diameter. Sections were stained as above but with rabbit anti-dystrophin (PAS-16734; Merino Fisher™ Scientific), mounted in Vectashield™+DAPI as above and imaged by confocal microscopy with a Zeiss™ 510 Duo microscope (Carl Zeiss™). DAPI labeling was evaluated with ImageJ™ (NIH, Bethesda, Md.), for determination of centrally nucleated fibers. Measurements of minimal Feret's diameter were obtained with Zeiss™ LSM Image Browser (Carl Zeiss™). A total of 312 myofibers from 5 Crym tg mice and 722 fibers from 5 control mice were analyzed.

### J. Measurements of Contractile Force

Nerve-evoked contractile function of gastrocnemius, extensor digitorum longus, or soleus muscles in vivo was evaluated as described. In brief, isofluorane-anesthetized mice were placed supine on a warming pad (37° C.) of an Aurora™ 1300 A system with knee position fixed and paw secured to the foot-plate of the 300C-FP. Percutaneous nerve stimulation was with brief (100 μecond) pulses with current adjusted to achieve maximal isometric force. The force vs frequency relationship was determined with 250 msecond trains of pulses between 1 and 150 Hz and normalized to muscle mass or cross-sectional area (CSA). Fatigability was determined by delivering tetanic trains (250 msecond at 80 Hz) every 2 seconds for 10 minutes for soleus or for 5 minutes for gastrocnemius. Data were analyzed with DMA-HT analysis software (Aurora Scientific™, Ontario, Canada) and evaluated for statistical difference via the Holm-S̆ídák test with GraphPad™ Prism version 8.2.0 for Mac (GraphPad™ Software, La Jolla, Calif.).

### K. Treadmill and Ad Libidum Running

Mice were conditioned to the treadmill over 3 days, as follows. Mice were placed in the treadmill with no belt movement for 10 minutes. The next day they were made to walk at 5 m/min for 10 minutes; this was increased to 10 m/minute the following day. For testing, mice were placed in the treadmill at a 7° incline. The speed was set at 10 m/minute and was increased by 1.5 m/minute every 2 minutes. Mice were considered exhausted when they were unable to run for 30 seconds consecutively. Each mouse was run 3 times with a minimum of 2 days rest between each run.

Additional Crym tg and control mice were housed in cages equipped with running wheels for 7 days. The number of times the wheel spun per minute was recorded every 6 minutes and analyzed to determine total distance run in different time periods, day and night.

### L. Protein Extraction

Tissues of interest (gastrocnemius, TA, soleus, diaphragm, heart, kidney, cerebral cortex and liver) were collected from 3-month old Crym tg and control mice, snap frozen in liquid nitrogen and stored at −80° C. Protein was extracted in RIPA Buffer (R0278-50ML, Sigma-Aldrich™), prepared with cOmplete Mini, EDTA-free protease inhibitor tablets (11836170001, Sigma-Aldrich™; 10 mL of RIPA buffer to every protease inhibitor tablet). Tissue was weighed and 100 μL per 10 mg of tissue of RIPA/protease inhibitor solution was added along with two 5 mm steel beads (69989; Qiagen™, Hilden, Germany). Samples were placed in a TissueLyser LT (Qiagen™) at 50 oscillations/second for 3 minutes, briefly vortexed, put on ice for 2 minutes, followed by a second round of 50 oscillations/second for 3 minutes in the TissueLyser. Samples were sonicated for 10 seconds and subjected to centrifugation at 12,470×g for 30 minutes at 4° C. The supernatant was transferred to a new microfuge tube and protein concentration was determined with BioRad™′s Protein Assay Dye Reagent Concentrate (Bradford Assay) (5000006, Bio-Rad™)

### M. Immunoblotting Protocols

Traditional Western blot: Muscle tissue was combined with an equal volume of sample homogenate and Laemmli sample buffer, containing 5% 2-mercaptoethanol, then further diluted in sample buffer to a final concentration of 1 mg/mL. Samples of 10 μg of Crym tg TA homogenate and 40 μg of control TA homogenate were loaded onto 4-12% Bis-Tris gels (NuPAGE NP0321PK2, Thermo Fisher™), electrophoresed for 1 h at 170 V, and transferred to nitrocellulose for 2 hours at 22 V. Blots were blocked for 3 hours in blocking solution (3% milk in TBS containing 0.1% Tween-20 and 10 mM azide). Monoclonal mouse anti-μ-crystallin (SC-376687, Santa Cruz Biotechnology™, Inc., Dallas, Tex.) and monoclonal rabbit anti-lysyl-tRNA synthetase antibodies (#129080, Abcam™, Cambridge, United Kingdom), used as a loading control, were diluted 1:1000 in blocking buffer and incubated overnight at room temperature. Lysyl-tRNA synthetase, encoded by the Kars gene, was used as the protein loading control because the expression of its mRNA showed minimal variance among mice and across genotypes, unlike many other common control proteins (see Example 9, below). At the time these studies were conducted, proteomic data was not available. After extensive washing, the membranes were incubated for 2 hours with HRP conjugated goat anti-rabbit (3-035-144, Jackson ImmunoResearch, West Grove, Pa.) or goat anti-mouse (47-035-146, Jackson ImmunoResearch) secondary antibody diluted 1:10,000 in blocking solution. After extensive washing, chemiluminescence was visualized with the Super Signal West Femto Maximum Sensitivity Kit (#34095, Thermo Fisher™) and imaged with a BioRad™ ChemiDoc XRS and image lab software (#1708265, BioRad™)

Capillary-based Western blot (Wes): Wes analysis was performed according to the manufacturer's instructions for the 12-230 kDa protein separation module (#SM-W004; Protein Simple, San Jose, Calif.) to provide an automated alternative to the traditional Western Blot. Instrument settings were as follows; stacking and separation time, 30 minutes; separation voltage, 375 V; time for antibody blocking, 30 minutes; incubation with primary antibody time, 60 minutes; incubation with secondary antibody time, 30 minutes; luminol/peroxide chemiluminescence detection time, 15 minutes (HDR exposure). Protein extracted from whole tissue lysates (gastrocnemius, TA, soleus, diaphragm, heart, kidney, cerebral cortex, quadriceps, liver) from 3 male Crym tg and 3 control were prepared with 2× Laemmli Sample Buffer (#1610737; Bio-Rad™, Hercules, Calif.) to generate an overall sample protein concentration of 1.0 mg/mL for each sample generated. The primary antibody to μ-crystallin (see above) was diluted 1:25 in Protein Simple's Antibody Diluent 2. A primary antibody to a control protein, aconitase-2 (ab129069; Abcam™, United Kingdom), was used at 1:200. We chose aconitase-2 as the protein loading control because this protein is abundantly expressed in many tissues, showed very little variability in expression in mice of the same genotype and between Crym tg and controls, as assayed by whole proteome LC.MS/MS (data not shown), and because the molecular mass of this protein is distinct from that of μ-crystallin. The primary and control antibodies were multiplexed. Samples were run as technical duplicates. Compass software (Compass for SW 4.0 Mac Beta; Protein Simple) was used to visualize the electropherograms and to analyze peaks of interest for area under the curve (AUC). The peak signal-to-noise ratio was set at >10 automatically by the software. Due to slight capillary-to-capillary variations in molecular weight readout, identified peaks at a molecular weight of interest were allowed a 10% range in molecular weight (as automatically set by the Wes system). AUC was analyzed with individual t-tests per tissue following the Benjamini, Krieger and Yekutieli FDR approach at Q=1%.

N. RNA and cDNA Preparation

Tissue from 3-month old Crym tg and control mice were removed, snap frozen in liquid nitrogen and stored at −80° C.

RNA was extracted using the following protocol. Tissue was placed in a 2 mL tube with 1 mL of TRIzol Reagent (Ser. No. 15/596,026; Thermo Fisher™ Scientific) and two 5 mm steel beads (69989; Qiagen™, Hilden, Germany), loaded into a TissueLyser LT (Qiagen™) and run at 50 oscillations/second for 2 minutes. Samples were checked for complete homogenization after 2 minutes and, if incompletely homogenized, run again in 30 second cycles with checks for complete homogenization after each cycle. Samples were vortexed briefly, inverted to mix at room temperature for 5 minutes, and subjected to centrifugation at 12,000 g for 10 minutes at 4° C. Supernatant was removed into a new 1.5 mL RNase-free tube and 200 μL of chloroform was added. After vortexing for 15 seconds, the sample remained at room temperature for 3 minutes. After a second centrifugation, at 12,000×g for 15 minutes at 4° C., the top (clear) layer was removed into a new 1.5 mL RNase free tube and 500 μL of ice-cold isopropanol was added. Tubes were vortexed and inverted several times to mix, and then left at room temperature for 10 minutes. Centrifugation at 12,000×g for 10 minutes at 4° C. generated a white pellet of RNA, which was washed with 1 mL of ice-cold 75% ethanol and collected again by centrifugation (7,500×g for 5 minutes at 4° C.). After drying at room temperature, the pellet was dissolved in 25-35 μL of RNase-, DNase-free water at 60° C. for 10 minutes. RNA samples were stored at −80° C. until use.

cDNA was generated with the QuantiTect™ Reverse Transcription Kit (205313; Qiagen). RT-qPCR was performed on a CFX Connect thermal cycler (Bio-Rad™) using the cDNA and PrimeTime Gene Expression Master Mix (1055772; IDT, Coralville, Iowa) in 20 μL reaction volumes in a BioRad™ CFX Connect thermal cycler following the manufacturer's protocol for the PrimeTime qPCR Probe Assays (IDT) except for the extension of the number of amplification cycles to 60. Primers are listed below (IDT PrimeTime RT-qPCR Probe-Based Assay) in Table 6, below. “ZEN” stands for the ZEN quencher and “3IABkFQ” stands for 3′ Iowa Black FQ. These are non-base modifiers that absorbs the light from fluorophores placed proximally to them. “HEX”, “SHEX”, and “FAM” are fluorophores that generate light when excited by particular wavelengths of light.

TABLE 6
Primers and Probes.

| | | | SEQ |
|---|---|---|---|
| | | | ID |
| Name | | Sequence | NO |
| Crym | Forward | 5′-GAGATGTTCGGGTCTGTTC | 5 |
| | | AT-3′ | |
| | Probe | 5′-/FAM/TCATCACAG/ZEN/ | 6 |
| | | TCACCATGGCAACAGA/ | |
| | | 3IABkFQ/-3′ | |
| | Reverse | 5′-GGCTTTACCCATTCACCAA | 7 |
| | | ATAA-3′ | |
| NomI | Forward | 5′-TAAACCCAGAGTTCACTTC | 8 |
| | | CTAC-3′ | |
| | Probe | 5′-/5HEX/TCGTCTTCA/ZEN/ | 9 |
| | | GTTTCCATCAACAGTGCA/ | |
| | | 3IABkFQ/-3′ | |
| | Reverse | 5′-CCTTCTCGCAACATTCCC | 10 |
| | | A-3′ | |
| Tert | Forward | 5′-CTCCTTTCCTCTAGGGCT | 11 |
| | | ATCT-3′ | |
| | Probe | 5′-/HEX/TCTCTGTCT/ZEN/ | 12 |
| | | CCCTTACCCACAGCT/ | |
| | | 3IABkFQ/-3′ | |
| | Reverse | 5′-AGTGCTGACATCTCATTCC | 13 |
| | | TTC-3′ | |

Relative tissue specific expression of Crym, standard deviations, and p values were calculated according to Taylor et al. Nom1 was used as the control gene to measure Crym expression by qPCR.

### P. Proteomic Comparison of Skeletal Muscle of Crym tg and Control Mice

Skeletal muscle samples were harvested from six 3-month-old, male, Crym tg and control littermate mice. All of the mice were perfused with cold PBS prior to muscle collection except for one of the 6 control samples. Samples, each comprising half of the left TA muscle, divided longitudinally, were homogenized by bead beating (see above). Sample preparation for proteomics and nano ultra-performance liquid chromatography-tandem mass spectrometry were performed as described in the art. Spectra were searched against a UniProt mouse reference proteome using Sequest HT algorithm described by Eng et al. and MS Amanda algorithm developed by Dorfer et al. Search configuration, false discovery control, and quantitation were as described.

### Q. RNA-seq

RNA extraction and RNA-seq was performed by Genewiz (South Plainfield, N.J.) on 20 mg samples of TA muscles from 3-month-old mice from 3 Crym tg littermate and 3 control littermate mice. Genewiz generated FASTQ files of the raw RNA-seq data, after removing transcripts that mapped to introns and transcripts that mapped to multiple genes. p-Values for comparisons of genes expressed in Crym tg and control mice were also from Genewiz.

### R. Ca²⁺ Transients

The amplitude of Ca²⁺ transients was recorded as described. In brief, isolated FDB fibers were loaded with Rhod-2AM (Thermo Fisher Scientific, Waltham, Mass.). Trains of voltage-induced Ca²⁺ transients were induced by field stimulation (A-M Systems, Carlsborg, Wash.). Rhod-2 was visualized with the Zeiss 510 Duo confocal microscope (Carl Zeiss). Image J 1.31v (NIH) was used for image analysis. The values reported were measured as the difference between maximal fluorescence intensity (Fₘₐₓ) and background fluorescence (F₀), normalized to Fₒ. Quantitative data are shown as mean±SE. A Student's t-test was used to compare the data with p<0.05 considered statistically significant.

### S. Metabolic Chambers

Data were from two different cohorts of 4 Crym tg and 4 C57BL/6J control mice housed in an Oxymax/CLAMS (Columbus Instruments, Columbus, Ohio) open circuit indirect calorimeter, in two separate experiments. Measurements for RER, Accumulated CO₂, Delta CO₂, CO₂ Out, Heat, Delta 02, Feed Weight, Volume CO₂, Accumulated 02, Feed Accumulation, Z Total, Volume 02, 02 Out, X Total, X Ambulatory, Flow, 02 In, and CO₂ In were taken directly or calculated every 18 minutes for approximately 92 hours.

Z Total and X Total refer to the total number of times an animal disrupted the infrared (IR) beam in the Z and X axis, respectively, while X Ambulatory refers to the number of times an animal disrupted at least two consecutive IR beams. Complete measurements were compiled and 24 hours' worth of measurements were analyzed, starting at the first dark cycle after a 24 hour period of acclimation. Values were normalized to total body weight and analyzed separately for light and dark periods. Outliers were identified with the ROUT method at Q=1%, with GraphPad™ Prism version 6.0e for Mac (GraphPad™ Software, La Jolla, Calif.). Averages were then calculated for all measurements except for Feed Weight, Feed Accumulation, Z Total, X Total, and X Ambulatory, for which the sums were determined. Each average or sum for each mouse was tested for significance, grouped as a genotype with an unpaired, parametric t-test with Welch's Correction.

### T. Diet Studies

Crym tg and control male and female mice at approximately 14 weeks old were placed on one of five diets. The number of mice on a particular diet varied from 5 to 16, due to the occasional death of a mouse, unrelated to the experiment, or to inability to weigh them on a particular date. The diets were “normal” diet (2018SX, Envigo Madison, Wis.), high fat diet (D12492), low fat diet (D12450J), high simple carbohydrate diet (D15040205), or high complex carbohydrate diet (D12450K), all from Research Diets Inc. (New Brunswick, N.J.) unless otherwise specified. Mouse weight (anaesthetized with 2.5% isoflurane) and food weight were recorded every Monday, Wednesday, and Friday for approximately 60 days. Water was provided ad libitum.

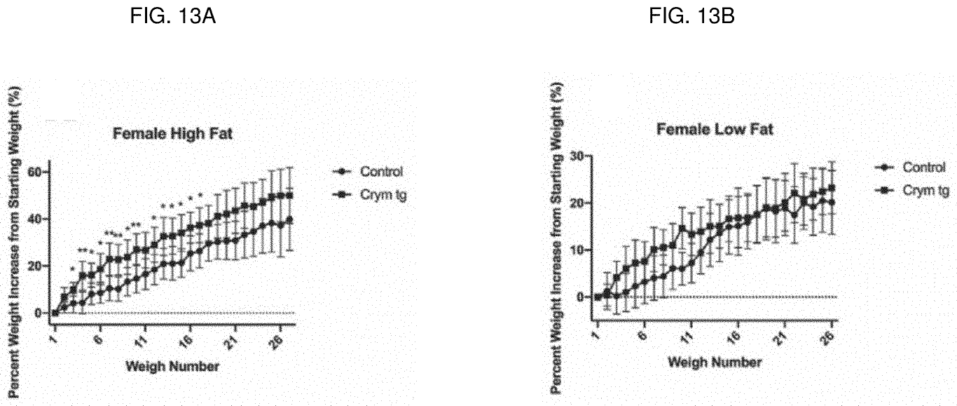

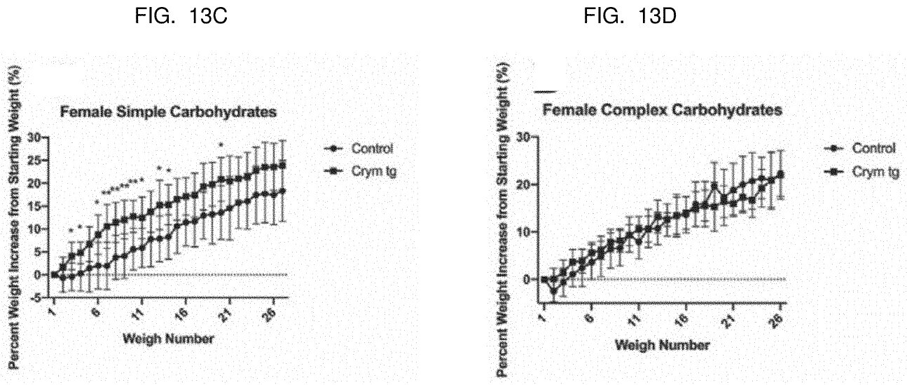

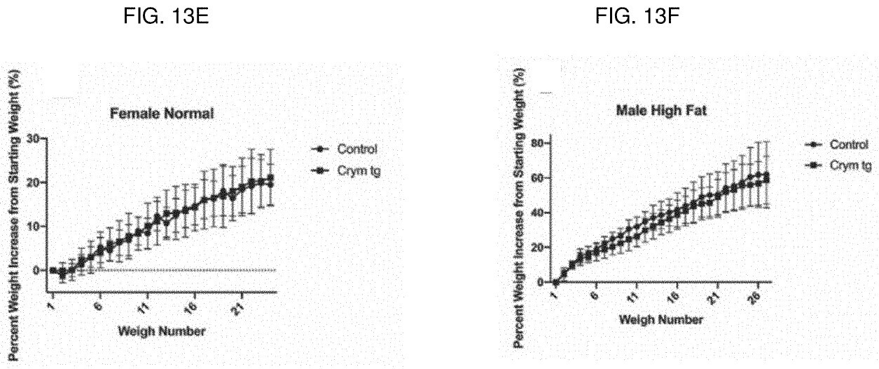

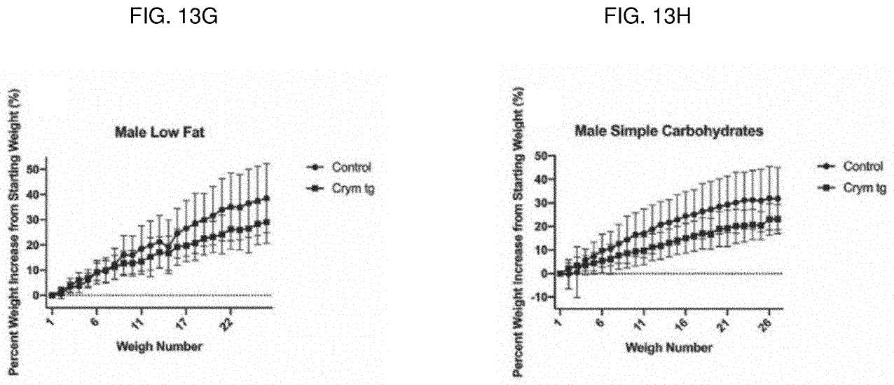

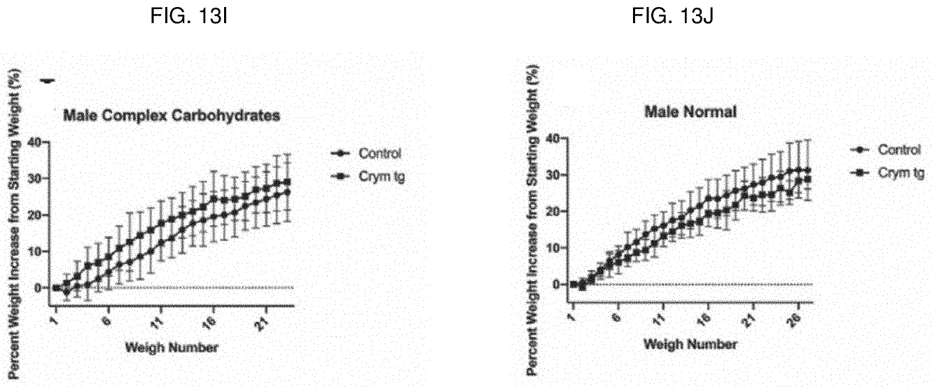

The results of this experiment show that female Crym tg mice placed on high fat or high simple carbohydrate diet gain weight faster than controls placed on the same diet. Male Crym tg mice placed on low fat or high simple carbohydrate diet trend towards gain weight more slowly compared to controls placed on the same diet. See FIG. 13. Mouse weights were normalized to their starting body weight. Using the Real Statistics Resource Pack software (Release 6.8) to run the Mauchly and John-Nagao-Sugiura tests. All of the groups violated at least one test for sphericity (α=0.05). Therefore, GraphPad™ Prism 8.2.0 for Mac was used to apply the Greenhouse and Geisser epsilon correction factor to either a two-way repeated measures (RM) ANOVA or a mixed-effects model, specifically a compound symmetry covariance matrix fit with Restricted Maximum Likelihood (REML) if data were missing for certain dates (α=0.05). When we observed significance with the two-way RM ANOVA or mixed-effects model (REML), we performed multiple post-hoc unpaired, two-tailed t tests assuming heteroscedasticity at α=0.05 to determine which data points showed significant differences between Crym tg and control mice.

U. T₃, T₄, TSH Levels

Homogenates of TA muscles and serum from four Crym tg and four control mice were sent to the clinical diagnostic service at Vanderbilt University (Nashville, Tenn.) for analysis of T₃, T₄ and TSH.

### V. Statistics

T₃, T₄ and TSH levels were analyzed by a two-tailed t-test with Welch's correction. The thyroid hormone mass (in ng/mL) was divided by total protein (in ng/mL) and used to determine the percent of total thyroid hormone per total protein in a milliliter of serum or homogenized muscle. The negative log base 10 of the percent of total protein for each value was determined and used to calculate the average and standard deviation.

### Example 2: Crym Transgenic Mice

A transgenic mouse in which murine Crym is expressed at high levels in skeletal muscle, comparable to those seen in some human muscle biopsies, was used to assess the effects an abundance of μ-crystallin would have on muscle structure and function, and on metabolism. The Crym transgene was encoded in a plasmid under the control of the human slow troponin I enhancer and the human skeletal actin promoter with the polyadenylation sequence of bovine growth hormone mRNA. (see FIG. 1A). After oocyte injection, the plasmid inserted randomly into intron 12 of the Cntn6 gene on murine chromosome 6 (see FIG. 1B), as determined by sequencing. The mice were bred to homozygosity and genotyping of the mice showed that there was a single insertion in the genome resulting in two copies of the Crym transgene in the diploid mouse genome. Chi-square statistics showed that observed counts of both male and female mice did not diverge from Mendelian expected counts in both genotype and sex for F1 mice that resulted from crossing Crym tg with control mice. Backcrossing Crym tg with control mice yielded the expected Mendelian inheritance pattern (see Table 7, below, which shows observed and expected control, heterozygote, and Crym tg mice counts according to a Mendelian inheritance pattern after backcrossing to generate F2 mice. The expected breakdown of mouse genotypes occurs in a classic 25%, 50%, 25% ratio for control, heterozygote, and Crym tg mice respectively in a total of 72 mice. Cntn6 was not significantly differentially expressed in control and Crym tg mice and showed little to no expression as assayed by RNA-seq in both. This indicates that the gene was not disrupted by the transgene's insertion.

TABLE 7
Mendelian Ratios of Offspring of Crym tg Heterozygotic Parents.

| | Observed | Expected |
|---|---|---|
| Control | 15 | 18 |
| Heterozygote | 40 | 36 |
| Crym tg | 16 | 18 |

### Example 3: RT-qPCR, Western Blot, and Immunofluorescence

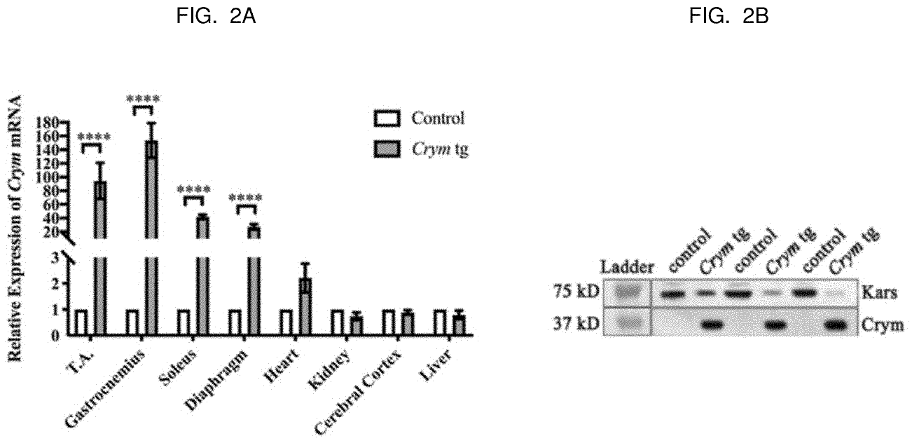

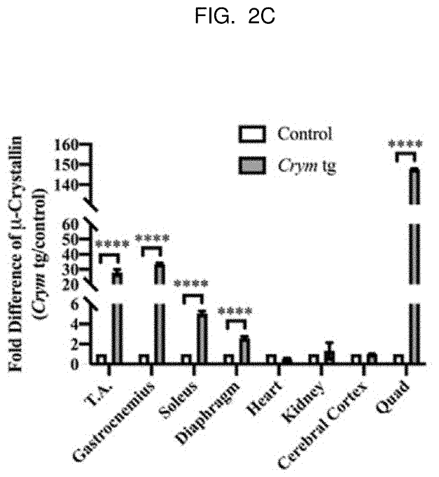

RT-qPCR of several skeletal muscles showed moderate to large increases in Crym mRNA in male Crym tg mice compared to male controls (see FIG. 2A). Briefly, Crym expression was measured in several skeletal muscles and other tissues from 3 Crym tg and 3 control mice. All analyses were in technical triplicate, except for soleus and diaphragm. Because many canonical control genes were significantly differentially expressed (DEG), RNA-seq and LC.MS/MS data were compared (Examples 9 and 10, respectively) to identify gene products with the smallest coefficient of variation. RefFinder™ was used to evaluate the most promising control genes among biological and technical replicates of various tissues (data not shown).

Nom1 was found to be optimal, showing the most stability. Using Nom1 mRNA as a standard, large increases in the Crym mRNA levels were observed in skeletal muscles of the Crym tg mice, compared both to controls and to heart muscle and non-muscle tissues. Notably, different skeletal muscles in the tg mice differed significantly in their relative expression of Crym. There were no significant differences in Crym mRNA levels in non-skeletal muscle tissues in tg and controls (see FIG. 2A).

Western blots were performed of μ-crystallin in Crym tg and control TA muscle extracts. Control mice had 40 μg of TA homogenate per lane, and Crym tg lanes had 10 μg of TA homogenates per lane. μ-crystallin was not detected reliably in controls in these blots. Immunoblotting confirmed that μ-crystallin (Crym) was present in elevated to highly elevated amounts in transgenic skeletal muscles compared to controls (see results in FIG. 2B).

Capillary-based immunoblotting in a Wes apparatus (FIG. 2C) was used to quantitate the differences, with aconitase 2 as a loading control. μ-Crystallin was quantitated with Protein Simple's Wes instrument. Areas under the curve, generated from electropherograms of μcrystallin expression, were normalized to aconitase-2 expression (n=3, in technical duplicate) in several muscles and other tissues. Multiple individual t-tests using the Benjamini, Krieger and Yekutieli FDR approach at Q=1% determined statistical significance (****=p<0.0001). The results show that Crym tg skeletal muscle express high levels of Crym mRNA and protein compared to controls and to other tissues analyzed. Specifically, the results showed that transgenic skeletal muscles contain 2.6 to 147.5-fold more μ-crystallin than controls, depending on the muscle. Remarkably, although all skeletal muscles we assayed showed increased levels of μ-crystallin, their relative amounts varied considerably (see FIG. 2C), consistent with the qRT-PCR results. These differences were highly significant (p<0.0001).

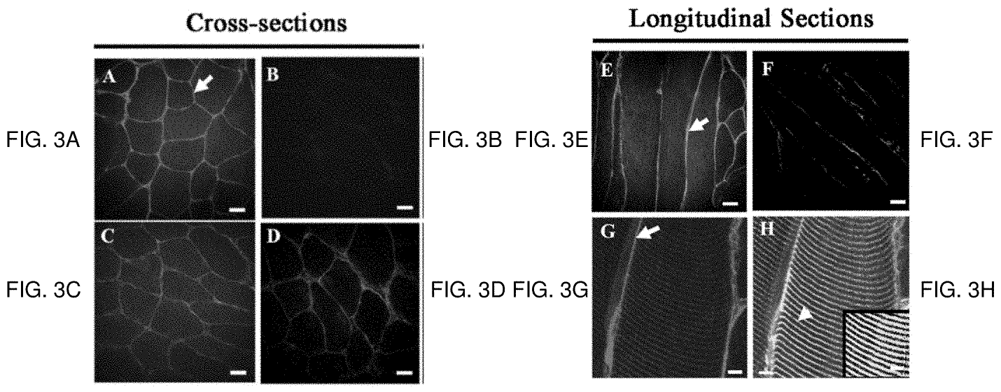

We also examined the distribution of μ-crystallin in transgenic muscle. Cross sections (FIG. 3A-FIG. 3D), longitudinal sections (FIG. 3E-FIG. 3H), of Crym tg (FIG. 3A, FIG. 3C, FIG. 3E, FIG. 3G, FIG. 3H) and control (FIG. 3B, FIG. 3D, FIG. 3F) TA (FIG. 3A, FIG. 3B, FIG. 3E-FIG. 3H) and soleus muscles (FIG. 3C, FIG. 3D) were stained with anti μ-crystallin antibody and Alexa Fluor-568-conjugated secondary antibody. In FIG. 3G-FIG. 3H, longitudinal sections of Crym tg TA muscle (FIG. 3G, μ-crystallin only) were colabeled with anti-desmin and Alexa Fluor-488-conjugated secondary antibody (FIG. 3H, yellow color shows colabeled structures). FIG. 3I and FIG. 3J: Flexor digitorum brevis (FDB) myofibers in culture from Crym tg (FIG. 3I) and control mice (FIG. 3J) were labeled with anti-μ-crystallin antibody and Alexa-Fluor 488-conjugated secondary antibody.

The results show that μ-crystallin was detected at higher levels in tg muscles than in controls. In TA muscle and FDB myofibers it was enriched at the levels of the sarcolemma (arrows, FIG. 3A, FIG. 3E, FIG. 3G, FIG. 3I) and Z-disks, colabeled with desmin (arrowhead, FIG. 3H) or shown without desmin colabel (arrowhead, FIG. 3I). Inset panels (FIG. 3H-FIG. 3J) are brightened and magnified. Crym tg (FIG. 3I) and control (FIG. 3J) FDB images were brightened and magnified equivalently. FIG. 3A-FIG. 3F, scale bars=20 μm; FIG. 3G, FIG. 3H, scale bars=5 μm; FIG. 3I, FIG. 3J, scale bars=10 μm.

In cross-sections of TA and flexor digitorum brevis (FDB) muscle, immunolabeling for μ-crystallin was concentrated near the sarcolemma (FIG. 3-3, arrows) but was also present in the myoplasm. Soleus cross sections did not show a similar concentration of μ-crystallin near the sarcolemma compared to controls (FIG. 3C, FIG. 3D), perhaps because they express lower amounts of the protein. Longitudinal sections of TA muscle and isolated FDB myofibers also showed high levels of μ-crystallin at or near the sarcolemma (FIG. 3E, FIG. 3G, FIG. 3I), but in addition revealed a faint striated distribution in the myoplasm (FIG. 3H, FIG. 3I), which in TA muscles colocalized with desmin at the level of the Z-disk (FIG. 3H, arrowhead). Immunolabeling for μ-crystallin was not observed in the capillaries or connective tissue surrounding myofibers. These results as well as results from qPCR and Wes data (FIG. 2A and FIG. 2C respectively) indicate that μ-crystallin is expressed at elevated levels in myofibers of the transgenic mice but not in other cell types in muscle or other tissues.

### Example 4: Hormone Levels

The levels of different hormones involved in thyroid hormone signaling in Crym tg and control mice were compared. Significantly, more T₃ in the TA muscle (about 190 fold more) of Crym tg mice and significantly less T₄ in the serum (˜1.2 fold less) compared to controls (p<0.001 and p<0.05, respectively). Thyroid stimulating hormone (TSH) was not significantly different in serum (see Table 1) of Crym tg and control mice and, as expected, was not detected in muscle. Intramuscular T₄ and serum T₃ also did not differ significantly between Crym tg and controls. Thus, the large increase in μ-crystallin in murine skeletal muscle is associated with an even larger increase in muscle T₃.

### Example 5: Morphology and Physiology

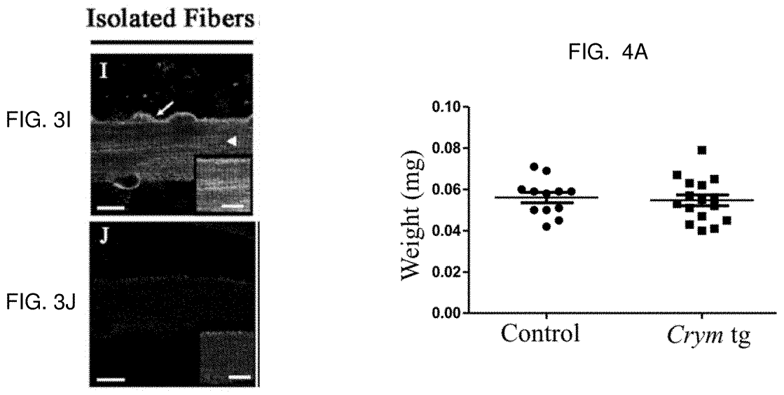

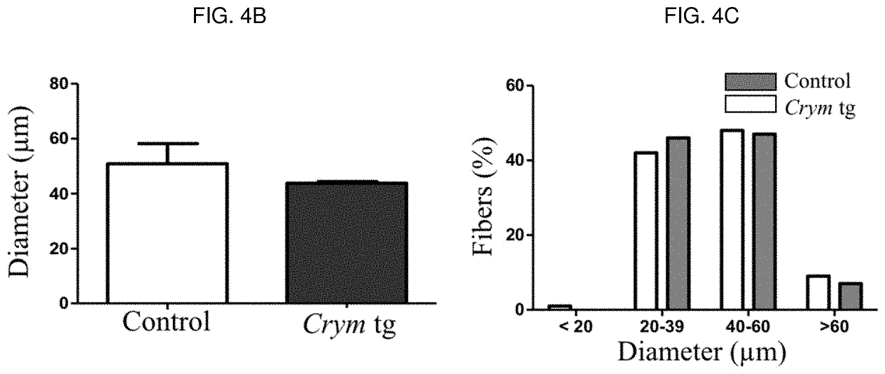

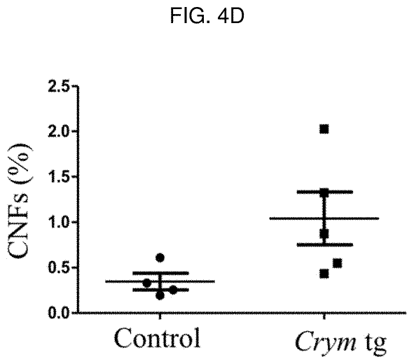

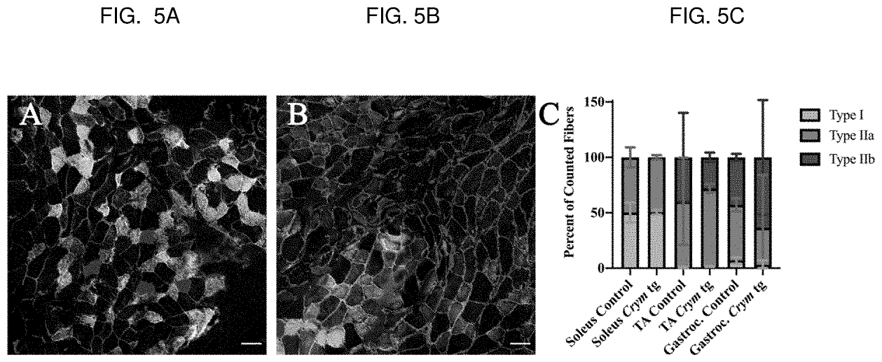

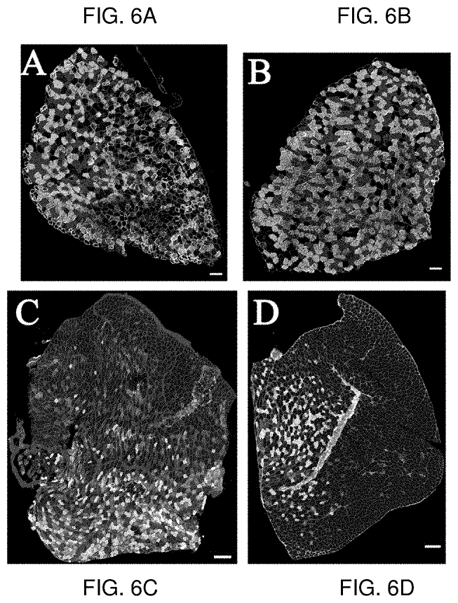

Different morphological and physiological assays of the structure and function of the transgenic mice showed no significant differences between Crym tgs and controls. The fiber sizes, distribution of fiber sizes, fiber types by immunohistochemistry of myosin heavy chains, weights, and the frequency of centrally nucleated fibers (CNFs) were not significantly altered in the TA muscles of Crym tgs (see FIG. 4, FIG. 5, and FIG. 6).

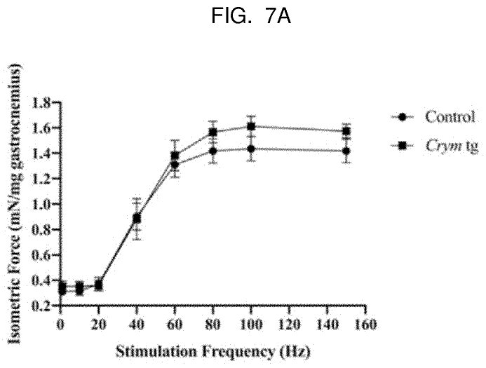

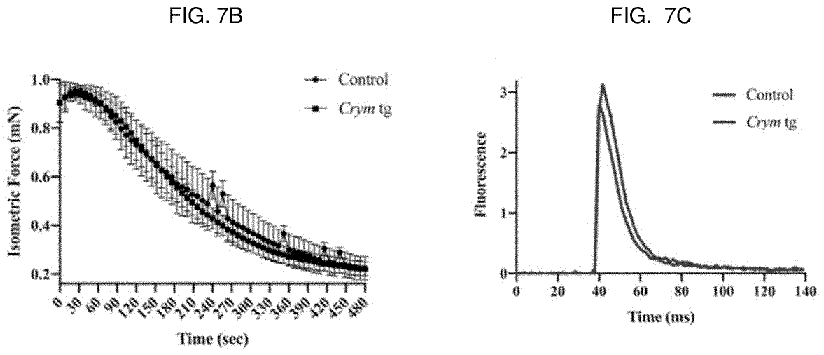

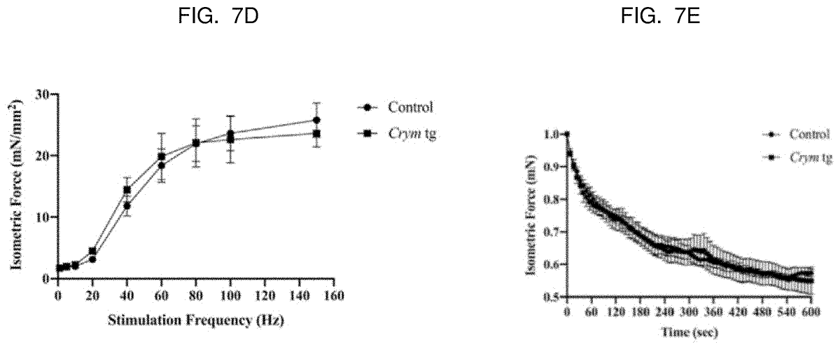

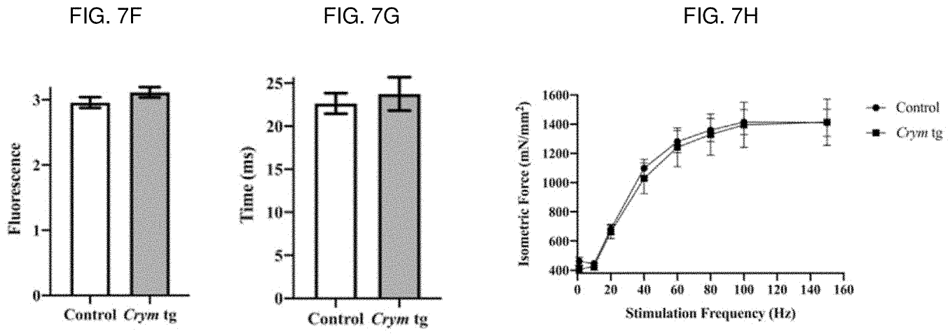

FIG. 7 presents data concerning contractile properties of Crym tg muscle, in particular force frequency curves (FIG. 7A, FIG. 7B, FIG. 7D, FIG. 7E, FIG. 7H) of gastrocnemius (FIG. 7A, FIG. 7B), soleus (FIG. 7D, FIG. 7E), and extensor digitorum longus (EDL) (FIG. 7H) muscle. Force was normalized to gastrocnemius muscle weight (FIG. 7A) or to soleus or EDL cross-sectional area (FIG. 7D and FIG. 7H). For fatigue, force was normalized to peak force (FIG. 7B and FIG. 7E). Ca²⁺ transients in isolated myofibers (FIG. 7C, FIG. 7F, and FIG. 7G). FIG. 7C shows representative Ca²⁺ transient evoked by a voltage pulse, visualized with Rhod2 (control and Crym tg, as indicated). FIG. 7F shows the maximal amplitudes of the Ca²⁺ transients. (n=100). FIG. 7G shows mean time constants for the decay of the transients (n=100). Statistics utilized the Student's t-test for Ca²⁺ measurements (FIG. 7F and FIG. 7G) or the Holm-S̆ídák test on the gastrocnemius (n=5; FIG. 7A, and FIG. 7B), soleus (control n=4, Crym tg n=5; FIG. 7D and FIG. 7E), or EDL (control n=6, Crym tg n=5; FIG. 7H) at α=0.05. All error bars show standard error.

The results show no statistically significant differences in any of these measurements. FIG. 7H shows force-frequency curves of isolated EDL muscles, normalized to cross sectional area. FIG. 7A shows force-frequency curves normalized to muscle weight in mg. FIG. 7B and FIG. 7E shows fatigue of contractile force over time. Specific force (measured in different ways in FIG. 7A vs. FIG. 7D and FIG. 7H) were not significantly different. Fatigue trended towards being slower in Crym tg gastrocnemius muscle (p<0.05, by Holm-S̆ídák). The results show no statistically significant differences in any of these measurements.

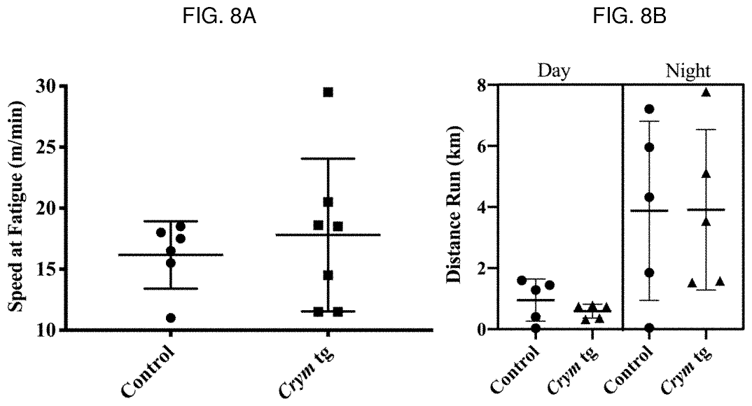

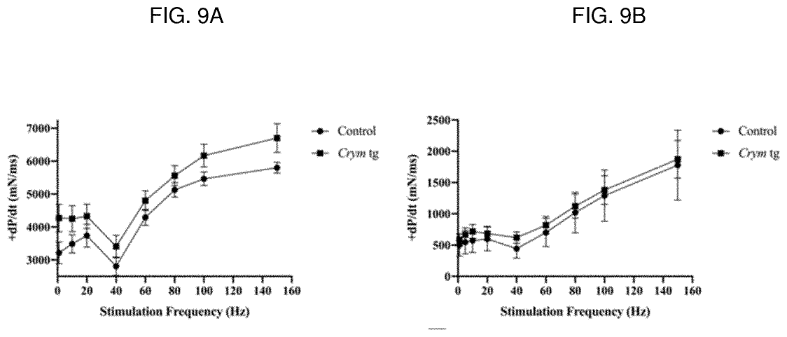

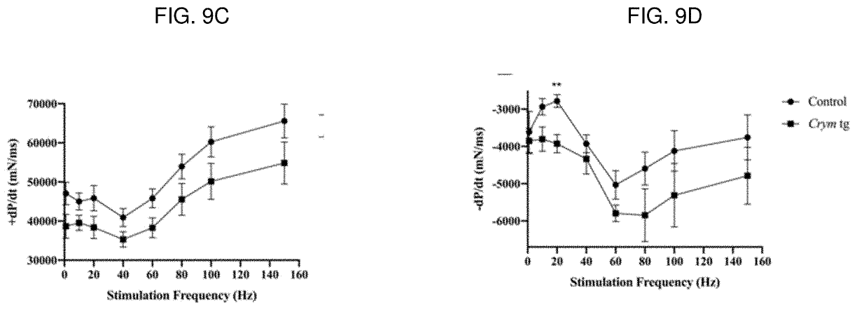

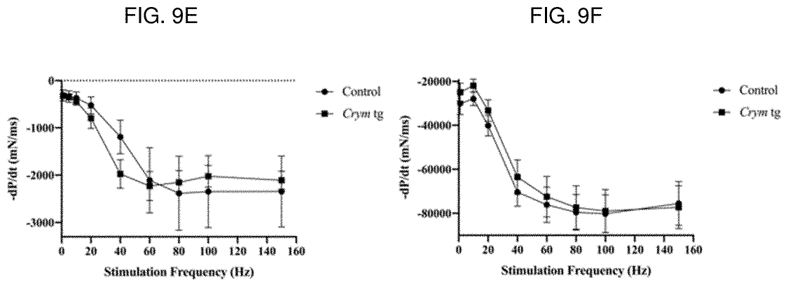

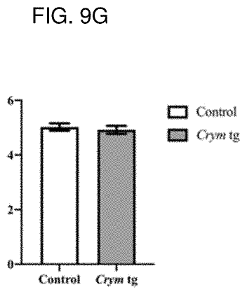

Specific isometric force of contraction, maximal rate of twitch force contraction/relaxation, grip strength, maximum treadmill running speed, and distance run also were indistinguishable between Crym tg and controls (FIG. 7A, FIG. 7B; FIG. 8; FIG. 9).

Fatiguability in the Crym tg mice showed a trend towards being slower than in controls, but this did not rise to the level of significance (FIG. 7B). At the single myofiber level, voltage-induced Ca²⁺ transients, maximal amplitudes of transients and transient decay rates, all measured with the Ca²⁺-sensitive fluorescent indicator, Rhod2, under conditions that measure changes in cytoplasmic and not mitochondrial Ca²⁺ (26), were identical in the two strains of mice (FIG. 7C, FIG. 7F, FIG. 7G).

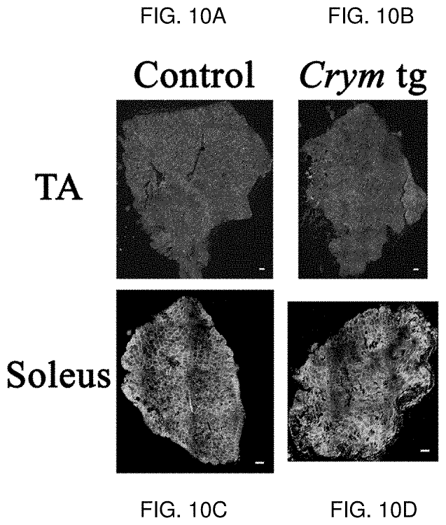

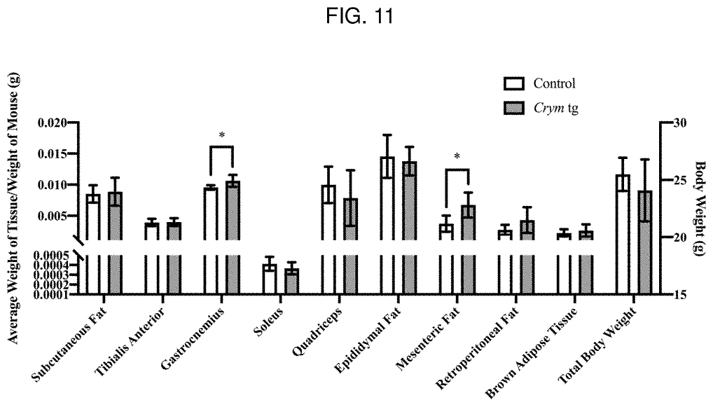

Fat staining with BODIPY (493/503) was also the same (FIG. 10). Tissues were dissected bilaterally and weighed together (except for subcutaneous fat and total body weight). Tissue weights were normalized to body weights. FIG. 11 shows the average weight various tissues and the total body weight of control mice and Crym tgs. Mesenteric fat and gastrocnemius muscle weighed significantly more in Crym tg compared to control mice as assayed by t test (*=p<0.05). All tissues (tibialis anterior, quadriceps, epididymal fat, mesenteric fat, brown adipose tissue, and total body weight) had a n=6 Crym tg and n=5 control except for subcutaneous fat (n=4 for both), soleus (control n=5, Crym tg n=6), and gastrocnemius and retroperitoneal fat (n=5 for both). Crym tg gastrocnemius and mesenteric fat showed small but significant increases compared to controls. See FIG. 11. No apparent differences were seen between Crym tg and control mice in intramuscular lipid content or distribution (n=5). However, Crym tg gastrocnemius muscles and mesenteric fat pads weighed slightly more than their control counterparts (p<0.05; FIG. 11).

The average weight of tibialis anterior, gastrocnemius, soleus, and quadriceps muscle, subcutaneous, epididymal, mesenteric, retroperitoneal, and brown adipose fat and the total body weight of control and Crym tg mice is shown in FIG. 11. See also FIG. 4B, which shows the fiber diameter; FIG. 4C, which shows the distribution of fiber diameters (%); FIG. 4D which shows centrally nucleated fibers (%); FIG. 5C which shows Type I, IIa, and IIb fibers (%); and FIG. 7F which shows fluorescence due to Ca⁺⁺ release stimulated by a single electrical pulse, measured with Rhod-2. For FIG. 4A, TA weight at 3 months age (Crym tg, n=8; control, n=6) is shown in FIG. 4A (two tailed Student's t-test with Welch's Correction (α=0.05)). FIG. 4B shows the fiber diameter (μm; mean±SD; n=3; two-tailed unpaired t-test). FIG. 4C shows the distribution of fiber diameters (%; two-tailed Fisher's Exact test (Crym tg n=259 fibers; Control n=720 fibers). In FIG. 4D, centrally nucleated fibers (CNF; %) are shown (a two-tailed unpaired t-test was performed (Crym tg n=5; Control n=4)). No significant differences were identified. None of the results show statistically significant differences between Crym tg and control mice. By these measures, the overexpression of Crym and the resulting accumulation of T₃ in myofibers has minimal effects on the structure or function of murine skeletal muscle.

### Example 6: Fiber Types

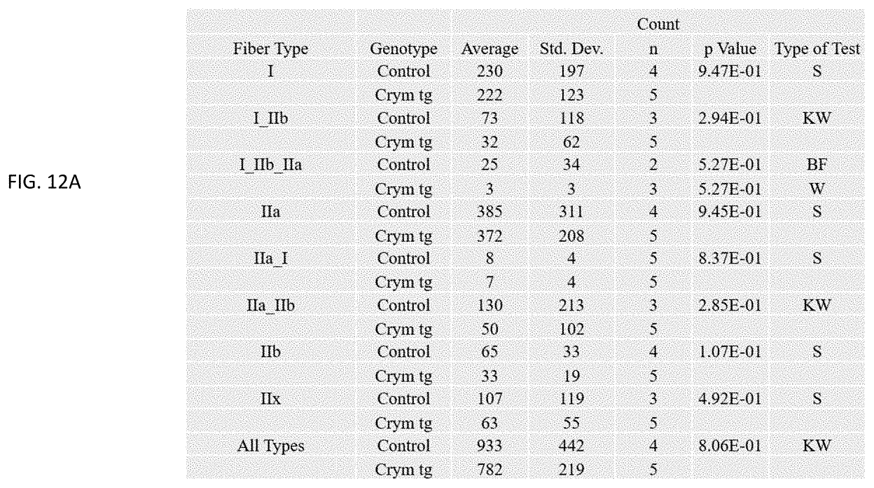

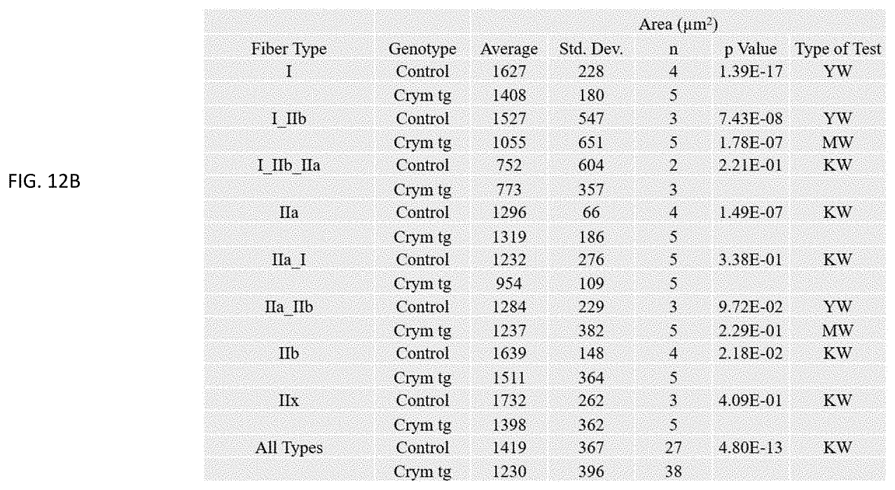

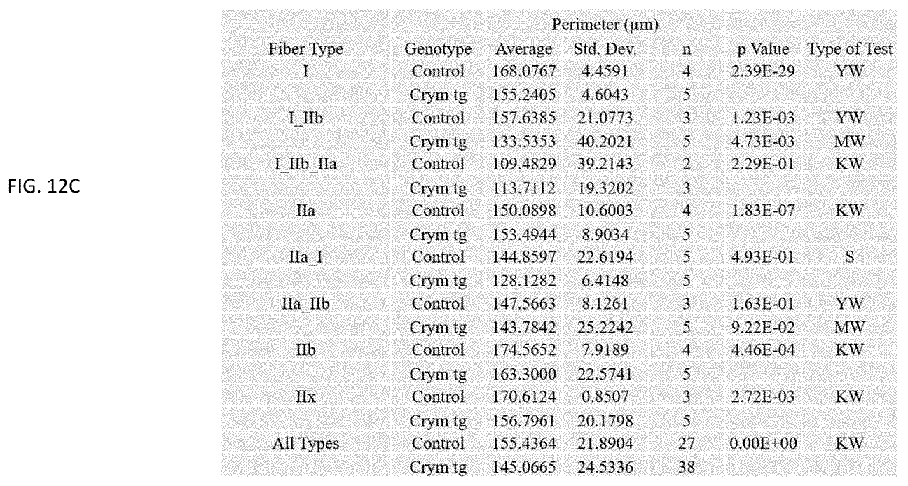

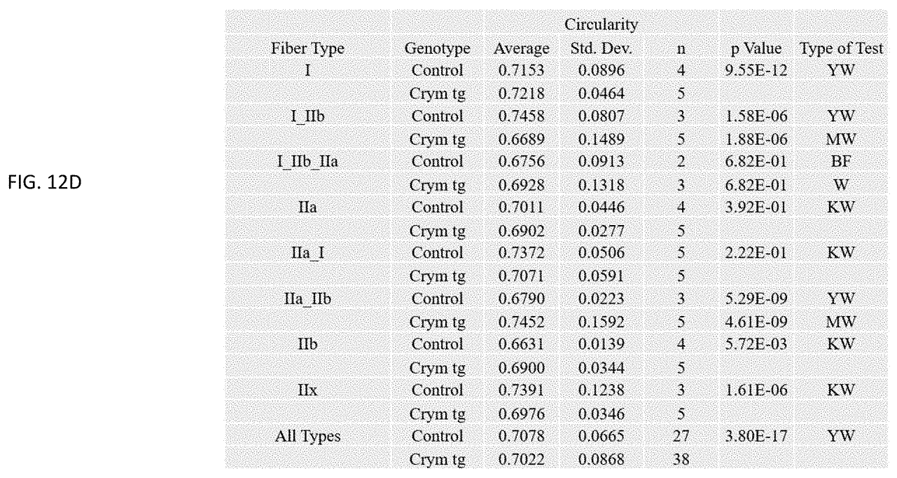

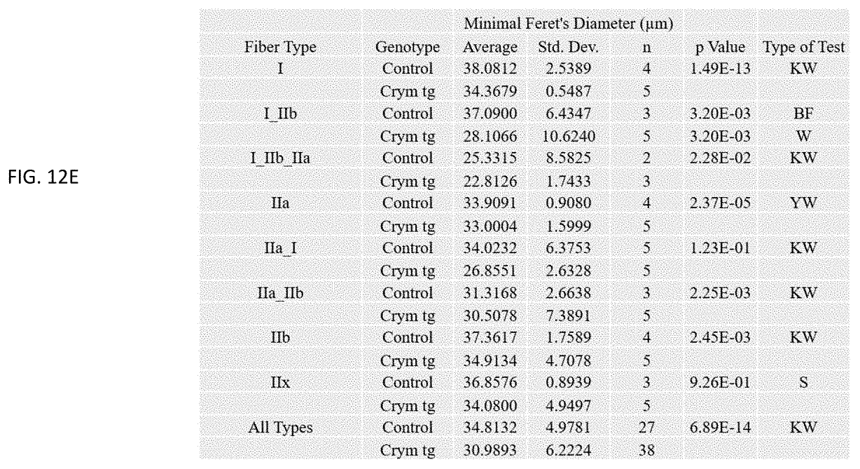

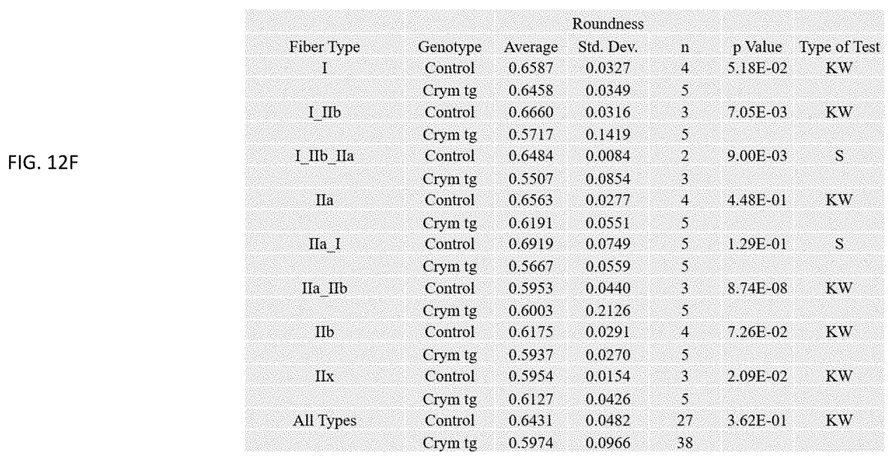

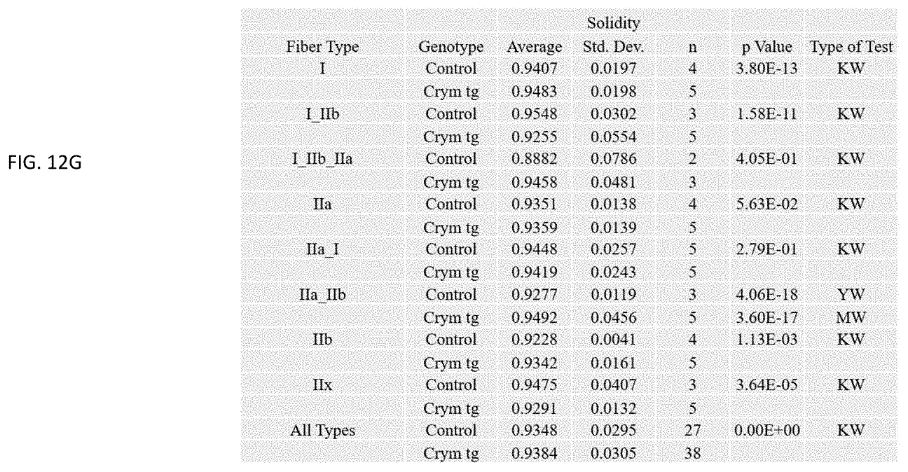

A machine learning approach was applied to obtain more quantitative information about the soleus muscles in the Crym tg mouse. No significant differences in the fiber type populations or total number of fibers of Crym tg soleus muscles (782±219 fibers) compared to controls (933±442). Furthermore, the results obtained by determining fiber type visually closely matched the results as determined computationally (FIG. 4C; FIG. 5C; FIG. 6A; FIG. 6B; FIG. 12). Significant differences in a few metrics were noted, however. In particular, the minimal Feret's diameter of Crym tg Type I, IIIb, IIIb/IIa, IIa, IIa/IIb, IIb, and all fiber types in aggregate were significantly smaller than control fibers of the same types while Type I/IIa and IIx fibers were not significantly altered in minimal Feret's diameter. Crym tg Type I, IIb, and IIa/IIb fibers were significantly more circular than controls, whereas Type IIIb, IIx, and all fiber types in the Crym tg in aggregate were less circular than controls (FIG. 12).

Using visual evaluation only, no significant differences in myosin-specific fiber type labeling in TA or gastrocnemius muscle cross sections were observed between Crym tg mice and controls (FIG. 5 and FIG. 6; the sizes of these muscles were too large to analyze by machine learning with our equipment). The results indicated that, although the fiber types in Crym tg and control soleus muscles are indistinguishable, several properties differ significantly between the two genotypes.

For FIG. 5 (Fiber types of Crym tg and control muscles), FIG. 5A shows a cross section of a control gastrocnemius stained with primary antibodies against various myosin heavy chain fiber type markers and laminin to visualize the cell membranes. Type I, Type IIa, Type IIb, and laminin are visible. FIG. 5B was prepared as in panel A, but with tg gastrocnemius muscle. Scale bars in FIG. 5A and FIG. 5B are 50 μm. FIG. 5C shows the percentage of fiber type distribution in three muscles, soleus, tibialis anterior (TA), and gastrocnemius (Gastroc.). A two tailed Fisher's Exact test was performed (Crym tg n=424, 323, 259; Control n=417, 318, 282; soleus, TA, and gastrocnemius fibers respectively). No statistically significant differences were found.

For FIG. 6 (soleus and TA muscle sections stained for three myosin heavy chain isoforms), Crym tg soleus (FIG. 6B) and TA (FIG. 6D) and control soleus (FIG. 6A) and TA (FIG. 6C) muscles were stained for Myh7 (Type I; BA-D5, isotype IgG2b, red), Myh2 (Type IIa; SC-71, isotype IgG1; green), and Myh4 (Type IIb; BF-F3, isotype IgM; blue). Wheat germ agglutinin (WGA; magenta) was used to stain the plasma membrane. No differences in myosin heavy chain staining were apparent in either soleus or TA cross sections comparing Crym tg and control mice (n=5).

STRING analysis of genes with altered expression in the Crym tg was performed. STRING was used to generate clusters of related genes. Disconnected nodes were removed and the interaction score was set to 0.90. MCL clustering with an inflation parameter of 3.0 was used to discern more similar gene clusters. The STRING network analysis showed large clusters of muscle-related genes and metabolically involved genes as well as smaller clusters of genes involved in ubiquitination, filament associated genes, the innate immune system.

### Example 7: Metabolism

Because T₃ can have a profound effect on metabolism, Crym tg and control mice were subjected to metabolic studies in Comprehensive Lab Monitoring System (CLAMS) cages. Eighteen metabolic traits were determined. Six and 4 traits were significantly different between Crym tg and control mice during the light and dark cycles, respectively. See Table 8, below. For this table, traits were measured in OSYMAX-CLAMS cages every 18 minutes for approximately 92 hours in 4 Crym tg and 4 control mice. Twenty-four hours of measurements were retained and segregated by light and dark cycle. The ROUT outlier method was used to remove obviously inaccurate measurement. A t test with Welch's Correction was used to compare Crym tg and control mice (*=p<0.05; **=p<0.005).

The respiratory exchange ratio (RER), a measure of the oxidative preference for carbohydrates vs fats, was significantly different during both the light and dark cycles, with essentially no change in the overall energy expenditure (Table 8). Table 8 shows the mean RER of the Crym tg and control mice, compared to standardized values. The results show that the oxidative metabolism in the transgenic mice has partially shifted away from the use of carbohydrates as an energy source and towards fats. The difference from controls was 13.7%. Other traits that also differed significantly between Crym tg and control mice in both light and dark periods were Delta CO₂, and Accumulated CO₂ (Table 8). Thus, overexpression of Crym in skeletal muscle has a significant effect on murine preference for fat as a fuel source for energy metabolism.

TABLE 8
Measured Metabolic Traits.

| | Dark Cycle | | Light Cycle | |
|---|---|---|---|---|
| | | P value | | P value |
| Measurement | P value | summary | P value | summary |
| RER | 0.0107 | * | 0.0019 | ** |
| Delta CO₂ | 0.0483 | * | 0.0100 | * |
| Accumulated CO₂ | 0.0489 | * | 0.0305 | * |
| CO₂ out | 0.0633 | ns | 0.0125 | * |
| Heat | 0.1244 | ns | 0.0480 | * |
| Delta O₂ | 0.1302 | ns | 0.0449 | * |
| Feed weight | 0.0305 | * | 0.0931 | ns |
| Accumulated O₂ | 0.1522 | ns | 0.1300 | ns |
| Volume CO₂ | 0.2580 | ns | 0.1002 | ns |
| O₂ out | 0.3313 | ns | 0.4596 | ns |
| Feed accumulation | 0.4068 | ns | 0.2860 | ns |
| Volume O₂ | 0.7370 | ns | 0.3824 | ns |
| X total | 0.8813 | ns | 0.4485 | ns |
| Flow | 0.8909 | ns | 0.9456 | ns |
| CO₂ in | 0.9796 | ns | 0.9963 | ns |
| X ambulatory | 0.9850 | ns | 0.1120 | ns |
| O₂ in | 0.9878 | ns | 0.9961 | ns |
| Z total | 0.9919 | ns | 0.9789 | ns |

### Example 8: Diet Study

Because the Crym tg mice prefer fats to carbohydrates as an energy source, Crym tg and control mice were fed high fat, low fat, high simple carbohydrate, high complex carbohydrate, and normal control diets. Their weight gain was measured over a period of 2 months. Specifically, Crym tg and control mice were placed on the different diets and weighed over the course of 60 days. See results in FIG. 13. Female (FIG. 13A-FIG. 13D) and male (FIG. 13F-FIG. 13I) Crym tg and control mice were placed on diet containing high fat (FIG. 13A, FIG. 13F), low fat (FIG. 13B, FIG. 13G), high simple carbohydrates (FIG. 13C, FIG. 13H), high complex carbohydrates (FIG. 13D, FIG. 13I), or on a normal diet (FIG. 13E, FIG. 13J) for 60 days. Mice were weighed three times a week and the percent weight increase from their starting weight was determined.

Significant increases in normalized body weight were seen for female Crym tg mice on the high simple carbohydrate and high fat diets, compared to controls. Female Crym tg mice on the other diets and male Crym tg mice on all 5 of the diets did not gain significantly more or less weight than controls (FIG. 13). There were no other significant results using a two-way repeated measures ANOVA or mixed-effects model (*=p<0.05, **=p<0.001, n=5-16).

### Example 9: Global Gene Expression (RNA-Seq)

The expression of a large number of genes can be affected by T₃ (see Table 1, above, for a list of genes with altered expression in Crym tg muscle that are also altered in hypothyroid or hyperthyroid conditions), which is thought to act largely through thyroid responsive elements (TREs). Since T₃ is present in much higher levels in Crym tg mice, RNA-seq was performed on TA muscle samples from 3 Crym tg and 3 control mice. More than 13,000 transcripts from approximately 8.4×10⁸ reads were measured. The samples had an average Q score>37 with approximately 87% of bases having a Q score greater than 30. After removing transcripts that aligned to introns or that mapped to multiple genes, and after Benjamini-Hochberg False Discovery Rate (FDR) correction, 566 genes were significantly different between Crym tg and controls at α=0.05 (see Table 9, below, a list of genes with altered expression in Crym tg muscle, determined by RNA-seq).

Gene ontology (GO) analysis revealed a relative decrease in the expression of genes encoding proteins involved in glycolysis and glycogen metabolism and in fast contractile speeds, and an increase in the expression of genes associated with oxidative metabolism and slower contraction (Table 3, Table 10). Ontological terms in Table 10 were derived from a list of 566 DEGs discovered via RNA-seq comparing Crym tg mice to controls. STRING network analysis also showed clusters of genes involved in ubiquitination, filament associated genes, and the innate immune system. After Benjamini-Hochberg FDR correction, DAVID chromosome annotation showed significantly more (p=5.7E-7) differentially expressed transcripts arising from chromosome 6 than would be expected. Notably, only 7 of the 566 DEGs contained putative TREs. See Table 2, above. For this table, a list of genes containing thyroid responsive elements (TREs) was generated from two computed TRE searches and one literatures review. This list of genes containing TREs which can act to promote or suppress the transcription of downstream genes in response to thyroid receptor complexes was cross referenced with 566 significant DEGs found via RNA-seq comparing the TA of Crym tg to the TA of control mice.

TABLE 9
Genes with Altered Expression in Crym tg Muscle, Determined by RNA-seq.

| Gene | | Gene | |
|---|---|---|---|
| Symbol | Gene Name | Symbol | Gene Name |
| Crym | crystallin, mu(Crym) | Lmcd1 | LIM and cysteine-rich domains 1(Lmcd1) |
| Gapdh | glyceraldehyde-3-phosphate | Dnaja4 | DnaJ heat shock protein family (Hsp40) member |
| | dehydrogenase(Gapdh) | | A4(Dnaja4) |
| Fhl1 | four and a half LIM domains 1(Fhl1) | Atp1b1 | ATPase, Na+/K+ transporting, beta 1 |
| | | | polypeptide(Atp1b1) |
| Lrrnl | leucine rich repeat protein 1, | Hspb7 | heat shock protein family, member 7 |
| | neuronal(Lrrn1) | | (cardiovascular)(Hspb7) |
| Actn2 | actinin alpha 2(Actn2) | H2-Q7 | histocompatibility 2, Q region locus 7(H2-Q7) |
| Myh2 | myosin, heavy polypeptide 2, skeletal | Mettl21c | methyltransferase like 21C(Mettl21c) |
| | muscle, adult(Myh2) | | |
| Gbe1 | glucan (1,4-alpha-), branching enzyme | Vldlr | very low density lipoprotein receptor(Vldlr) |
| | 1(Gbe1) | | |
| Cryab | crystallin, alpha B(Cryab) | Hspb6 | heat shock protein, alpha-crystallin-related, |
| | | | B6(Hspb6) |
| Myoz2 | myozenin 2(Myoz2) | Slc16a1 | solute carrier family 16 (monocarboxylic acid |
| | | | transporters), member 1(Slc16a1) |
| Amd1 | S-adenosylmethionine decarboxylase | Kbtbd12 | kelch repeat and BTB (POZ) domain containing |
| | 1(Amd1) | | 12(Kbtbd12) |
| Car14 | carbonic anhydrase 14(Car14) | Tspan12 | tetraspanin 12(Tspan12) |
| H2-Q6 | histocompatibility 2, Q region locus 6(H2- | Ybx3 | Y box protein 3(Ybx3) |
| | Q6) | | |
| Eepd1 | endonuclease/exonuclease/phosphatase | Nrep | neuronal regeneration related protein(Nrep) |
| | family domain containing 1(Eepd1) | | |
| Slc40a1 | solute carrier family 40 (iron-regulated | Cops7a | COP9 signalosome subunit 7A(Cops7a) |
| | transporter), member 1(Slc40a1) | | |
| Pik3r1 | phosphatidylinositol 3-kinase, regulatory | Padi2 | peptidyl arginine deiminase, type II(Padi2) |
| | subunit, polypeptide 1 (p85 alpha)(Pik3r1) | | |
| Ehd4 | EH-domain containing 4(Ehd4) | Tbc1d1 | TBC1 domain family, member 1(Tbc1d1) |
| Slc41a3 | solute carrier family 41, member | Myh1 | myosin, heavy polypeptide 1, skeletal muscle, |
| | 3(Slc41a3) | | adult(Myh1) |
| Smox | spermine oxidase(Smox) | Itpr1 | inositol 1,4,5-trisphosphate receptor 1(Itpr1) |
| Slc15a5 | solute carrier family 15, member | Dynit1b | dynein light chain Tctex-type 1B(Dynit1b) |
| | 5(Slc15a5) | | |
| Actr3b | ARP3 actin-related protein 3B(Actr3b) | Abra | actin-binding Rho activating protein(Abra) |
| Myoz1 | myozenin 1(Myoz1) | Ptpn3 | protein tyrosine phosphatase, non-receptor type |
| | | | 3(Ptpn3) |
| Stbd1 | starch binding domain 1(Stbd1) | Htra4 | HtrA serine peptidase 4(Htra4) |
| Wbscr17 | Williams-Beuren syndrome chromosome | Amot | angiomotin(Amot) |
| | region 17 homolog (human)(Wbscr17) | | |
| Ckmt2 | creatine kinase, mitochondrial 2(Ckmt2) | Cdk19 | cyclin-dependent kinase 19(Cdk19) |
| Ube2d1 | ubiquitin-conjugating enzyme E2D1 | Flnc | filamin C, gamma(Flnc) |
| | (Ube2d1) | | |
| 9330159F19Rik | RIKEN cDNA 9330159F19 | Egln3 | egl-9 family hypoxia-inducible factor 3(Egln3) |
| | gene(9330159F19Rik) | | |
| Aldh1a1 | aldehyde dehydrogenase family 1, | Tmem233 | transmembrane protein 233(Tmem233) |
| | subfamily A1(Aldh1a1) | | |
| Pde8a | phosphodiesterase 8A(Pde8a) | Slc30a2 | solute carrier family 30 (zinc transporter), |
| | | | member 2(Slc30a2) |
| Spns2 | spinster homolog 2(Spns2) | G0s2 | G0/G1 switch gene 2(G0s2) |
| Ip6k3 | inositol hexaphosphate kinase 3(Ip6k3) | Cpeb1 | cytoplasmic polyadenylation element binding |
| | | | protein 1(Cpeb1) |
| Pvalb | parvalbumin(Pvalb) | Cacna2d4 | calcium channel, voltage-dependent, alpha |
| | | | 2/delta subunit 4(Cacna2d4) |
| Ankrd2 | ankyrin repeat domain 2 (stretch | Stom | stomatin(Stom) |
| | responsive muscle)(Ankrd2) | | |
| Eci1 | enoyl-Coenzyme A delta isomerase | Casp12 | caspase 12(Casp12) |
| | 1(Eci1) | | |
| Plcd4 | phospholipase C, delta 4(Plcd4) | H2-Q4 | histocompatibility 2, Q region locus 4(H2-Q4) |
| A930018M24Rik | | Slc25a25 | solute carrier family 25 (mitochondrial carrier, |
| | | | phosphate carrier), member 25(Slc25a25) |
| H2-D1 | histocompatibility 2, D region locus 1(H2- | Col7a1 | collagen, type VII, alpha 1(Col7a1) |
| | D1) | | |
| Calm3 | calmodulin 3(Calm3) | Stat5b | signal transducer and activator of transcription |
| | | | 5B(Stat5b) |
| Mgst1 | microsomal glutathione S-transferase | Adck3 | coenzyme Q8A(Coq8a) |
| | 1(Mgst1) | | |
| Ociad2 | OCIA domain containing 2(Ociad2) | Klhl33 | kelch-like 33(Klhl33) |
| Mlf1 | myeloid leukemia factor 1(M1f1) | Sbk2 | SH3-binding domain kinase family, member |
| | | | 2(Sbk2) |
| Tmem65 | transmembrane protein 65(Tmem65) | Ankrd23 | ankyrin repeat domain 23(Ankrd23) |
| Slc38a10 | solute carrier family 38, member | Plekhb1 | pleckstrin homology domain containing, family |
| | 10(Slc38a10) | | B (evectins) member 1(Plekhb1) |
| Myod1 | myogenic differentiation 1(Myod1) | Abhd18 | abhydrolase domain containing 18(Abhd18) |
| Trim63 | tripartite motif-containing 63(Trim63) | Adamtsl4 | ADAMTS-like 4(Adamtsl4) |
| Zfand5 | zinc finger, AN1-type domain 5(Zfand5) | Dgat2 | diacylglycerol O-acyltransferase 2(Dgat2) |
| Kcnn3 | potassium intermediate/small conductance | | |
| | calcium-activated channel, subfamily N, | Npc1 | Niemann-Pick type C1(Npc1) |
| | member 3(Kcnn3) | | |
| Adh1 | alcohol dehydrogenase 1 (class I)(Adh1) | Kcnc1 | potassium voltage gated channel, Shaw-related |
| | | | subfamily, member 1(Kcnc1) |
| Nr1d1 | nuclear receptor subfamily 1, group D, | P2ry2 | purinergic receptor P2Y, G-protein coupled |
| | member 1(Nr1d1) | | 2(P2ry2) |
| Smim10l1 | small integral membrane protein 10 like | Ugp2 | UDP-glucose pyrophosphorylase 2(Ugp2) |
| | 1(Smim10l1) | | |
| Rbfox1 | RNA binding protein, fox-1 homolog (C. | Homer2 | homer scaffolding protein 2(Homer2) |
| | elegans) 1(Rbfox1) | | |
| Slc47a1 | solute carrier family 47, member | Ak4 | adenylate kinase 4(Ak4) |
| | 1(Slc47a1) | | |
| Sorbs2 | sorbin and SH3 domain containing | Mdh1 | malate dehydrogenase 1, NAD (soluble)(Mdh1) |
| | 2(Sorbs2) | | |
| Apol6 | apolipoprotein L 6(Apol6) | Ltbr | lymphotoxin B receptor(Ltbr) |
| Gpr19 | G protein-coupled receptor 19(Gpr19) | Plpp1 | phospholipid phosphatase 1(Plpp1) |
| Emp1 | epithelial membrane protein 1(Emp1) | Klhl31 | kelch-like 31(Klhl31) |
| Tmod4 | tropomodulin 4(Tmod4) | Pcx | pyruvate carboxylase(Pcx) |
| Mustn1 | musculoskeletal, embryonic nuclear | Slc43a1 | solute carrier family 43, member 1(Slc43a1) |
| | protein 1(Mustn1) | | |
| Slc2a3 | solute carrier family 2 (facilitated glucose | Bpgm | 2,3-bisphosphoglycerate mutase(Bpgm) |
| | transporter), member 3(Slc2a3) | | |
| Irf5 | interferon regulatory factor 5(Irf5) | Neto2 | neuropilin (NRP) and tolloid (TLL)-like |
| | | | 2(Neto2) |
| Spryd7 | SPRY domain containing 7(Spryd7) | Actn3 | actinin alpha 3(Actn3) |
| Myot | myotilin(Myot) | Srpr | signal recognition particle receptor (‘docking |
| | | | protein’)(Srpr) |
| Vim | vimentin(Vim) | Ssx2ip | synovial sarcoma, X breakpoint 2 interacting |
| | | | protein(Ssx2ip) |
| P2ry1 | purinergic receptor P2Y, G-protein | Dusp18 | dual specificity phosphatase 18(Dusp18) |
| | coupled 1(P2ry1) | | |
| Cbfb | core binding factor beta(Cbfb) | Ralgapa2 | Ral GTPase activating protein, alpha subunit 2 |
| | | | (catalytic)(Ralgapa2) |
| Nos1 | nitric oxide synthase 1, neuronal(Nos1) | Asb15 | ankyrin repeat and SOCS box-containing |
| | | | 15(Asb15) |
| Gm44386 | | Nmrk2 | nicotinamide riboside kinase 2(Nmrk2) |
| Rad52 | RAD52 homolog, DNA repair | Myh4 | myosin, heavy polypeptide 4, skeletal |
| | protein(Rad52) | | muscle(Myh4) |
| Hfe2 | hemochromatosis type 2 (juvenile)(Hfe2) | Acss2 | acyl-CoA synthetase short-chain family member |
| | | | 2(Acss2) |
| Ddit41 | DNA-damage-inducible transcript 4- | Trafd1 | TRAF type zinc finger domain containing |
| | like(Ddit41) | | 1(Trafd1) |
| Klhl34 | kelch-like 34(Klhl34) | Got2 | glutamatic-oxaloacetic transaminase 2, |
| | | | mitochondrial(Got2) |
| Fmo2 | flavin containing monooxygenase 2(Fmo2) | Myl12a | myosin, light chain 12A, regulatory, non- |
| | | | sarcomeric(Myl12a) |
| Tmod1 | tropomodulin 1(Tmod1) | Csrp3 | cysteine and glycine-rich protein 3(Csrp3) |
| Esr1 | estrogen receptor 1 (alpha)(Esr1) | Pla2g12a | phospholipase A2, group XIIA(Pla2g12a) |
| Timm44 | translocase of inner mitochondrial | Prkag3 | protein kinase, AMP-activated, gamma 3 non- |
| | membrane 44(Timm44) | | catatlytic subunit(Prkag3) |
| Tiparp | TCDD-inducible poly(ADP-ribose) | Dlst | dihydrolipoamide S-succinyltransferase (E2 |
| | polymerase(Tiparp) | | component of 2-oxo-glutarate complex)(Dlst) |
| Dapp1 | dual adaptor for phosphotyrosine and 3- | Tead4 | TEA domain family member 4(Tead4) |
| | phosphoinositides 1(Dapp1) | | |
| Btg2 | B cell translocation gene 2, anti- | Sar1b | secretion associated Ras related GTPase |
| | proliferative(Btg2) | | 1B(Sar1b) |
| Rps6ka5 | ribosomal protein S6 kinase, polypeptide | Ntng2 | netrin G2(Ntng2) |
| | 5(Rps6ka5) | | |
| Tmem63a | transmembrane protein 63a(Tmem63a) | Iqsec2 | IQ motif and Sec7 domain 2(Iqsec2) |
| Rgcc | regulator of cell cycle(Rgcc) | Mospd1 | motile sperm domain containing 1(Mospd1) |
| Ndufa9 | NADH dehydrogenase (ubiquinone) 1 | Cyp27a1 | cytochrome P450, family 27, subfamily a, |
| | alpha subcomplex, 9(Ndufa9) | | polypeptide 1(Cyp27a1) |
| Sec24a | Sec24 related gene family, member A (S. | Rab12 | RAB12, member RAS oncogene family(Rab12) |
| | cerevisiae)(Sec24a) | | |
| Fhod1 | formin homology 2 domain containing | Ank2 | ankyrin 2, brain(Ank2) |
| | 1(Fhod1) | | |
| Nid1 | nidogen 1(Nid1) | 2310040G24Rik | RIKEN cDNA 2310040G24 |
| | | | gene(2310040G24Rik) |
| Scn4b | sodium channel, type IV, beta(Scn4b) | Maf | avian musculoaponeurotic fibrosarcoma |
| | | | oncogene homolog(Maf) |
| Ttll4 | tubulin tyrosine ligase-like family, member | Wfs1 | Wolfram syndrome 1 homolog (human)(Wfs1) |
| | 4(Ttll4) | | |
| Thnsl2 | threonine synthase-like 2 | Alpl | alkaline phosphatase, liver/bone/kidney(Alpl) |
| | (bacterial)(Thnsl2) | | |
| 9830004L10Rik | | Zfp703 | zinc finger protein 703(Zfp703) |
| Ddx47 | DEAD (Asp-Glu-Ala-Asp) box | Ppargc1a | peroxisome proliferative activated receptor, |
| | polypeptide 47(Ddx47) | | gamma, coactivator 1 alpha(Ppargc1a) |
| B2m | beta-2 microglobulin(B2m) | Rhot2 | ras homolog family member T2(Rhot2) |
| Zfp9 | zinc finger protein 9(Zfp9) | Rsrp1 | arginine/serine rich protein 1(Rsrp1) |
| Mcm2 | minichromosome maintenance complex | Alas1 | aminolevulinic acid synthase 1(Alas1) |
| | component 2(Mcm2) | | |
| Npnt | nephronectin(Npnt) | Sqstm1 | sequestosome 1(Sqstm1) |
| Lsmem1 | leucine-rich single-pass membrane protein | Tmem246 | transmembrane protein 246(Tmem246) |
| | 1(Lsmem1) | | |
| Borcs5 | BLOC-1 related complex subunit | Myom3 | myomesin family, member 3(Myom3) |
| | 5(Borcs5) | | |
| Lynx1 | Ly6/neurotoxin 1(Lynx1) | Deptor | DEP domain containing MTOR-interacting |
| | | | protein(Deptor) |
| Fam131a | family with sequence similarity 131, | Amd2 | S-adenosylmethionine decarboxylase 2(Amd2) |
| | member A(Fam131a) | | |
| Chmp4c | charged multivesicular body protein | Slc22a4 | solute carrier family 22 (organic cation |
| | 4C(Chmp4c) | | transporter), member 4(Slc22a4) |
| Pim1 | proviral integration site 1(Pim1) | Mlec | malectin(Mlec) |
| Wnt5b | wingless-type MMTV integration site | Tulp3 | tubby-like protein 3(Tulp3) |
| | family, member 5B(Wnt5b) | | |
| Mrpl35 | mitochondrial ribosomal protein | Mrln | myoregulin(Mrln) |
| | L35(Mrpl35) | | |
| Mib1 | mindbomb E3 ubiquitin protein ligase | Tmem140 | transmembrane protein 140(Tmem140) |
| | 1(Mib1) | | |
| Bhlhe41 | basic helix-loop-helix family, member | Lpl | lipoprotein lipase(Lpl) |
| | e41(Bhlhe41) | | |
| Raver2 | ribonucleoprotein, PTB-binding 2(Raver2) | Tln1 | talin 1(Tln1) |
| Xpr1 | xenotropic and polytropic retrovirus | Hk2 | hexokinase 2(Hk2) |
| | receptor 1(Xpr1) | | |
| Acadvl | acyl-Coenzyme A dehydrogenase, very | Lamb2 | laminin, beta 2(Lamb2) |
| | long chain(Acadvl) | | |
| Park7 | Parkinson disease (autosomal recessive, | Trim24 | tripartite motif-containing 24(Trim24) |
| | early onset) 7(Park7) | | |
| Slc25a11 | solute carrier family 25 (mitochondrial | Osbpl3 | oxysterol binding protein-like 3(Osbpl3) |
| | carrier oxoglutarate carrier), member | | |
| | 11(Slc25a11) | | |
| Vgll2 | vestigial like family member 2(Vgll2) | Eif4ebp1 | eukaryotic translation initiation factor 4E |
| | | | binding protein 1(Eif4ebp1) |
| Gpt2 | glutamic pyruvate transaminase (alanine | Pgm2 | phosphoglucomutase 2(Pgm2) |
| | aminotransferase) 2(Gpt2) | | |
| Ldhb | lactate dehydrogenase B(Ldhb) | Kcnq5 | potassium voltage-gated channel, subfamily Q, |
| | | | member 5(Kcnq5) |
| Abcg2 | ATP-binding cassette, sub-family G | Rnf114 | ring finger protein 114(Rnf114) |
| | (WHITE), member 2(Abcg2) | | |
| Dbp | D site albumin promoter binding | Sub1 | SUB1 homolog (S. cerevisiae)(Sub1) |
| | protein(Dbp) | | |
| Tmtc1 | transmembrane and tetratricopeptide repeat | Slc12a4 | solute carrier family 12, member 4(Slc12a4) |
| | containing 1(Tmtc1) | | |
| Leo1 | Leo1, Paf1/RNA polymerase II complex | Etl4 | enhancer trap locus 4(Etl4) |
| | component(Leo1) | | |
| Msrb1 | methionine sulfoxide reductase B1(Msrb1) | Slc37a4 | solute carrier family 37 (glucose-6-phosphate |
| | | | transporter), member 4(Slc37a4) |
| Igfn1 | immunoglobulin-like and fibronectin type | Stac3 | SH3 and cysteine rich domain 3(Stac3) |
| | III domain containing 1(Igfnl) | | |
| Ctsf | cathepsin F(Ctsf) | Adamtsl5 | ADAMTS-like 5(Adamtsl5) |
| Atp1a1 | ATPase, Na+/K+ transporting, alpha 1 | Mgea5 | meningioma expressed antigen 5 |
| | polypeptide(Atp1a1) | | (hyaluronidase)(Mgea5) |
| Gbp10 | guanylate-binding protein 10(Gbp10) | Phkb | phosphorylase kinase beta(Phkb) |
| H19 | H19, imprinted maternally expressed | Lrp6 | low density lipoprotein receptor-related protein |
| | transcript(H19) | | 6(Lrp6) |
| A330023F24Rik | RIKEN cDNA A330023F24 | Hist1h4i | histone cluster 1, H4i(Hist1h4i) |
| | gene(A330023F24Rik) | | |
| Anks1 | ankyrin repeat and SAM domain | Pnck | pregnancy upregulated non-ubiquitously |
| | containing 1(Anks1) | | expressed CaM kinase(Pnck) |
| Gm5113 | predicted gene 5113(Gm5113) | Slc43a3 | solute carrier family 43, member 3(Slc43a3) |
| Myoz3 | myozenin 3(Myoz3) | Nmt2 | N-myristoyltransferase 2(Nmt2) |
| Ldb3 | LIM domain binding 3(Ldb3) | Tmem106b | transmembrane protein 106B(Tmem106b) |
| Pygm | muscle glycogen phosphorylase(Pygm) | Myl3 | myosin, light polypeptide 3(Myl3) |
| Setd7 | SET domain containing (lysine | Asb14 | ankyrin repeat and SOCS box-containing |
| | methyltransferase) 7(Setd7) | | 14(Asb14) |
| Aak1 | AP2 associated kinase 1(Aak1) | Tagln | transgelin(Tagln) |
| Fzd5 | frizzled class receptor 5(Fzd5) | Slc16a10 | solute carrier family 16 (monocarboxylic acid |
| | | | transporters), member 10(Slc16a10) |
| Fry | FRY microtubule binding protein(Fry) | Tead1 | TEA domain family member 1(Tead1) |
| Cbr2 | carbonyl reductase 2(Cbr2) | Ppara | peroxisome proliferator activated receptor |
| | | | alpha(Ppara) |
| Irs2 | insulin receptor substrate 2(Irs2) | Myh10 | myosin, heavy polypeptide 10, non- |
| | | | muscle(Myh10) |
| Hnrnph1 | heterogeneous nuclear ribonucleoprotein | Epas1 | endothelial PAS domain protein 1(Epas1) |
| | H1 (Hnrnph1) | | |
| Anapc5 | anaphase-promoting complex subunit | Tfcp2l1 | transcription factor CP2-like 1(Tfcp2l1) |
| | 5(Anapc5) | | |
| Col9a1 | collagen, type IX, alpha 1(Col9a1) | Hbp1 | high mobility group box transcription factor |
| | | | 1(Hbp1) |
| Rnf150 | ring finger protein 150(Rnf150) | Fmo1 | flavin containing monooxygenase 1(Fmo1) |
| Tiam1 | T cell lymphoma invasion and metastasis | Bace1 | beta-site APP cleaving enzyme 1(Bace1) |
| | 1(Tiam1) | | |
| Got1 | glutamic-oxaloacetic transaminase 1, | Ogt | O-linked N-acetylglucosamine (GlcNAc) |
| | soluble(Got1) | | transferase (UDP-N- |
| | | | acetylglucosamine:polypeptide-N- |
| | | | acetylglucosaminyl transferase)(Ogt) |
| Car3 | carbonic anhydrase 3(Car3) | Tigar | Trp53 induced glycolysis repulatory |
| | | | phosphatase(Tigar) |
| Polr1a | polymerase (RNA) I polypeptide | Ciapin1 | cytokine induced apoptosis inhibitor 1(Ciapin1) |
| | A(Polr1a) | | |
| Mgll | monoglyceride lipase(Mgll) | Fam234b | family with sequence similarity 234, member |
| | | | B(Fam234b) |
| Atp2b3 | ATPase, Ca++ transporting, plasma | Lrrc38 | leucine rich repeat containing 38(Lrrc38) |
| | membrane 3(Atp2b3) | | |
| Ndufb5 | NADH dehydrogenase (ubiquinone) 1 beta | March7 | membrane-associated ring finger (C3HC4) |
| | subcomplex, 5(Ndufb5) | | 7(March7) |
| Rtn4 | reticulon 4(Rtn4) | Slc16a3 | solute carrier family 16 (monocarboxylic acid |
| | | | transporters), member 3(Slc16a3) |
| Perm1 | PPARGC1 and ESRR induced regulator, | Cux2 | cut-like homeobox 2(Cux2) |
| | muscle 1(Perm1) | | |
| Mtx2 | metaxin 2(Mtx2) | Fzd7 | frizzled class receptor 7(Fzd7) |
| Smyd1 | SET and MYND domain containing | Gadd45b | growth arrest and DNA-damage-inducible 45 |
| | 1(Smyd1) | | beta(Gadd45b) |
| Plpp7 | phospholipid phosphatase 7 | Lum | lumican(Lum) |
| | (inactive)(Plpp7) | | |
| C1s1 | complement component 1, s subcomponent | Fhl3 | four and a half LIM domains 3(Fhl3) |
| | 1(C1s1) | | |
| Bag3 | BCL2-associated athanogene 3(Bag3) | Oxnad1 | oxidoreductase NAD-binding domain containing |
| | | | 1(Oxnad1) |
| Ttc19 | tetratricopeptide repeat domain 19(Ttc19) | Mga | MAX gene associated(Mga) |
| Fam213b | family with sequence similarity 213, | Slc38a3 | solute carrier family 38, member 3(Slc38a3) |
| | member B(Fam213b) | | |
| Neu2 | neuraminidase 2(Neu2) | Kctd20 | potassium channel tetramerisation domain |
| | | | containing 20(Kctd20) |
| Akap11 | A kinase (PRKA) anchor protein | Mybpc2 | myosin binding protein C, fast-type(Mybpc2) |
| | 11(Akap11) | | |
| Myh11 | myosin, heavy polypeptide 11, smooth | Sfxn3 | sideroflexin 3(Sfxn3) |
| | muscle(Myh11) | | |
| Lmod2 | leiomodin 2 (cardiac)(Lmod2) | Smarca2 | SWI/SNF related, matrix associated, actin |
| | | | dependent regulator of chromatin, subfamily a, |
| | | | member 2(Smarca2) |
| Pdlim1 | PDZ and LIM domain 1 (elfin)(Pdlim1) | Suclg2 | succinate-Coenzyme A ligase, GDP-forming, |
| | | | beta subunit(Suclg2) |
| Zxdc | ZXD family zinc finger C(Zxdc) | Slc16a2 | solute carrier family 16 (monocarboxylic acid |
| | | | transporters), member 2(Slc16a2) |
| Tmem52 | transmembrane protein 52(Tmem52) | Cfd | complement factor D (adipsin)(Cfd) |
| Ndufs8 | NADH dehydrogenase (ubiquinone) Fe-S | Sorl1 | sortilin-related receptor, LDLR class A repeats- |
| | protein 8(Ndufs8) | | containing(Sorl1) |
| Me1 | malic enzyme 1, NADP(+)-dependent, | Dok7 | docking protein 7(Dok7) |
| | cytosolic(Me1) | | |
| Nqo1 | NAD(P)H dehydrogenase, quinone | Ly6c1 | lymphocyte antigen 6 complex, locus C1(Ly6c1) |
| | 1(Nqo1l) | | |
| Fam195a | family with sequence similarity 195, | Atp5g1 | ATP synthase, H+ transporting, mitochondrial |
| | member A(Fam195a) | | F0 complex, subunit C1 (subunit 9)(Atp5g1) |
| Psmd8 | proteasome (prosome, macropain) 26S | Gramd1b | GRAM domain containing 1B(Gramd1b) |
| | subunit, non-ATPase, 8(Psmd8) | | |
| Cd36 | CD36 antigen(Cd36) | Carnmt1 | carnosine N-methyltransferase 1(Carnmt1) |
| Vps72 | vacuolar protein sorting 72(Vps72) | Apbb2 | amyloid beta (A4) precursor protein-binding, |
| | | | family B, member 2(Apbb2) |
| D930015E06Rik | RIKEN cDNA D930015E06 | Chd2 | chromodomain helicase DNA binding protein |
| | gene(D930015E06Rik) | | 2(Chd2) |
| H2-K1 | histocompatibility 2, K1, K region(H2-K1) | Ttll7 | tubulin tyrosine ligase-like family, member |
| | | | 7(Ttll7) |
| Kalrn | kalirin, RhoGEF kinase(Kalrn) | Pde7a | phosphodiesterase 7A(Pde7a) |
| Hebp1 | heme binding protein 1(Hebp1) | Psme4 | proteasome (prosome, macropain) activator |
| | | | subunit 4(Psme4) |
| Amigo1 | adhesion molecule with Ig like domain | Chrnb1 | cholinergic receptor, nicotinic, beta polypeptide |
| | 1(Amigo1) | | 1 (muscle)(Chrnb1) |
| Tbc1d16 | TBC1 domain family, member | Nfkbia | nuclear factor of kappa light polypeptide gene |
| | 16(Tbc1d16) | | enhancer in B cells inhibitor, alpha(Nfkbia) |
| Gpr157 | G protein-coupled receptor 157(Gpr157) | Mylk4 | myosin light chain kinase family, member |
| | | | 4(Mylk4) |
| Actg1 | actin, gamma, cytoplasmic 1(Actg1l) | Mt1 | metallothionein 1(Mt1) |
| Slc25a26 | solute carrier family 25 (mitochondrial | Adamts8 | a disintegrin-like and metallopeptidase |
| | carrier, phosphate carrier), member | | (reprolysin type) with thrombospondin type 1 |
| | 26(Slc25a26) | | motif, 8(Adamts8) |
| Zranb1 | zinc finger, RAN-binding domain | Lgalsl | lectin, galactoside binding-like(Lgalsl) |
| | containing 1(Zranb1) | | |
| Adck1 | aarF domain containing kinase 1(Adck1) | Ppif | peptidylprolyl isomerase F (cyclophilin F)(Ppif) |
| | solute carrier family 41, member | Col8a1 | collagen, type VIII, alpha 1(Col8a1) |
| Slc41a1 | 1(Slc41a1) | | |
| Atl2 | atlastin GTPase 2(Atl2) | Ptprk | protein tyrosine phosphatase, receptor type, |
| | | | K(Ptprk) |
| Acaca | acetyl-Coenzyme A carboxylase | Gpt | glutamic pyruvic transaminase, soluble(Gpt) |
| | alpha(Acaca) | | |
| Murc | muscle-related coiled-coil protein(Murc) | Klhl38 | kelch-like 38(Klhl38) |
| Ddi2 | DNA-damage inducible protein 2(Ddi2) | Zfp956 | zinc finger protein 956(Zfp956) |
| Sorbs1 | sorbin and SH3 domain containing | Rhobtb3 | Rho-related BTB domain containing 3(Rhobtb3) |
| | 1 (Sorbs1) | | |
| Serpinb6a | serine (or cysteine) peptidase inhibitor, | Sema3b | sema domain, immunoglobulin domain (Ig), |
| | clade B, member 6a(Serpinb6a) | | short basic domain, secreted, (semaphorin) |
| | | | 3B(Sema3b) |
| Hhatl | hedgehog acyltransferase-like(Hhatl) | Tuba8 | tubulin, alpha 8(Tuba8) |
| D1Ertd622e | DNA segment, Chr 1, ERATO Doi 622, | B3galnt2 | UDP-GalNAc:betaGlcNAc beta 1,3- |
| | expressed(D1Ertd622e) | | galactosaminyltransferase, polypeptide |
| | | | 2(B3galnt2) |
| Pcbp1 | poly(rC) binding protein 1(Pcbp1) | Ccrl2 | chemokine (C-C motif) receptor-like 2(Ccrl2) |
| Macf1 | microtubule-actin crosslinking factor | Itga7 | integrin alpha 7(Itga7) |
| | 1(Macf1) | | |
| Fbxo40 | F-box protein 40(Fbxo40) | Ky | kyphoscoliosis peptidase(Ky) |
| Ccdc50 | coiled-coil domain containing 50(Ccdc50) | Mapre2 | microtubule-associated protein, RP/EB family, |
| | | | member 2(Mapre2) |
| Rcl1 | RNA terminal phosphate cyclase-like | Rhbdl1 | rhomboid, veinlet-like 1 (Drosophila)(Rhbdl1) |
| | 1(Rcl1) | | |
| Myo1e | myosin IE(Myo1e) | Ptges2 | prostaglandin E synthase 2(Ptges2) |
| Wnt4 | wingless-type MMTV integration site | Plekhb2 | pleckstrin homology domain containing, family |
| | family, member 4(Wnt4) | | B (evectins) member 2(Plekhb2) |
| Dusp13 | dual specificity phosphatase 13(Dusp13) | Mtfp1 | mitochondrial fission process 1(Mtfp1) |
| Gid4 | GID complex subunit 4, VID24 | Rnf126 | ring finger protein 126(Rnf126) |
| | homolog(Gid4) | | |
| Tnip1 | TNFAIP3 interacting protein 1(Tnip1) | Igfbp5 | insulin-like growth factor binding protein |
| | | | 5(Igfbp5) |
| Pick1 | protein interacting with C kinase 1(Pick1) | Gpx3 | glutathione peroxidase 3(Gpx3) |
| Sdhaf4 | succinate dehydrogenase complex | Pik3ip1 | phosphoinositide-3-kinase interacting protein |
| | assembly factor 4(Sdhaf4) | | 1(Pik3ip1) |
| Fnip1 | folliculin interacting protein 1(Fnip1) | Prdx2 | peroxiredoxin 2(Prdx2) |
| Vegfb | vascular endothelial growth factor | Nudt8 | nudix (nucleoside diphosphate linked moiety |
| | B(Vegfb) | | X)-type motif 8(Nudt8) |
| Spsb1 | splA/ryanodine receptor domain and | Paqr7 | progestin and adipoQ receptor family member |
| | SOCS box containing 1(Spsb1) | | VII(Paqr7) |
| Cnnm4 | cyclin M4(Cnnm4) | Lrg1 | leucine-rich alpha-2-glycoprotein 1(Lrg1) |
| Zfp275 | zinc finger protein 275(Zfp275) | Esrrb | estrogen related receptor, beta(Esrrb) |
| Aldh2 | aldehyde dehydrogenase 2, | Ddr1 | discoidin domain receptor family, member |
| | mitochondrial(Aldh2) | | 1(Ddr1) |
| Akap8l | A kinase (PRKA) anchor protein 8- | Nup210 | nucleoporin 210(Nup210) |
| | like(Akap8l) | | |
| Agl | amylo-1,6-glucosidase, 4-alpha- | Prdx5 | peroxiredoxin 5(Prdx5) |
| | glucanotransferase(Agl) | | |
| Rhob | ras homolog family member B(Rhob) | Srsf5 | serine/arginine-rich splicing factor 5(Srsf5) |
| Strbp | spermatid perinuclear RNA binding | Ogdh | oxoglutarate (alpha-ketoglutarate) |
| | protein(Strbp) | | dehydrogenase (lipoamide)(Ogdh) |
| Rbm24 | RNA binding motif protein 24(Rbm24) | Fam57b | family with sequence similarity 57, member |
| | | | B(Fam57b) |
| Fgf1 | fibroblast growth factor 1(Fgf1) | Fermt2 | fermitin family member 2(Fermt2) |
| Noct | nocturnin(Noct) | Rtn4ip1 | reticulon 4 interacting protein 1(Rtn4ip1) |
| Camta1 | calmodulin binding transcription activator | Marveld1 | MARVEL (membrane-associating) domain |
| | 1(Camta1) | | containing 1(Marveld1) |
| Sema6c | sema domain, transmembrane domain | Pnpla2 | patatin-like phospholipase domain containing |
| | (TM), and cytoplasmic domain, | | 2(Pnpla2) |
| | (semaphorin) 6C(Sema6c) | | |
| Lgals1 | lectin, galactose binding, soluble 1(Lgals1) | Ogg1 | 8-oxoguanine DNA-glycosylase 1(Ogg1) |
| Snx12 | sorting nexin 12(Snx12) | Phka1 | phosphorylase kinase alpha 1(Phka1) |
| Ccdc91 | coiled-coil domain containing 91(Ccdc91) | Cdc42ep2 | CDC42 effector protein (Rho GTPase binding) |
| | | | 2(Cdc42ep2) |
| Dlat | dihydrolipoamide S-acetyltransferase (E2 | Dnase1l1 | deoxyribonuclease 1-like 1(Dnase1l1) |
| | component of pyruvate dehydrogenase | | |
| | complex)(Dlat) | | |
| Atf7ip | activating transcription factor 7 interacting | Adprhl1 | ADP-ribosylhydrolase like 1(Adprhl1) |
| | protein(Atf7ip) | | |
| Cobll1 | Cobl-like 1(Cobll1) | Ctsb | cathepsin B(Ctsb) |
| Atp1a2 | ATPase Na+/K+ transporting, alpha 2 | Pdha1 | pyruvate dehydrogenase E1 alpha 1(Pdha1) |
| | polypeptide(Atp1a2) | | |
| Sdhd | succinate dehydrogenase complex, subunit | Tns1 | tensin 1(Tns1) |
| | D, integral membrane protein(Sdhd) | | |
| Hmcn2 | hemicentin 2(Hmcn2) | Ttc9 | tetratricopeptide repeat domain 9(Ttc9) |
| Fat3 | FAT atypical cadherin 3(Fat3) | Rgs5 | regulator of G-protein signaling 5(Rgs5) |
| Sdhb | succinate dehydrogenase complex, subunit | Gpcpd1 | glycerophosphocholine phosphodiesterase |
| | B, iron sulfur (Ip)(Sdhb) | | 1(Gpcpd1) |
| Gpd1 | glycerol-3-phosphate dehydrogenase 1 | Rmi2 | RecQ mediated genome instability 2(Rmi2) |
| | (soluble)(Gpd1) | | |
| Ccng1 | cyclin G1(Ccng1) | Fam21 | family with sequence similarity 21(Fam21) |
| Notch3 | notch 3(Notch3) | Slc45a4 | solute carrier family 45, member 4(Slc45a4) |
| Amotl1 | angiomotin-like 1(Amotl1) | Amer1 | APC membrane recruitment 1(Amer1) |
| Casq1 | calsequestrin 1(Casq1) | Phkg1 | phosphorylase kinase gamma 1(Phkg1) |
| Pdhb | pyruvate dehydrogenase (lipoamide) | Lgmn | legumain(Lgmn) |
| | beta(Pdhb) | | |
| 2310015K22Rik | | P2rx5 | purinergic receptor P2X, ligand-gated ion |
| | | | channel, 5(P2rx5) |
| Celf2 | CUGBP, Elav-like family member | Dennd5a | DENN/MADD domain containing 5A(Dennd5a) |
| | 2(Celf2) | | |
| Rian | maternally Expressed S. Small Nucleolar | Gclc | glutamate-cysteine ligase, catalytic |
| | RNA Host Gene(Rian) | | subunit(Gclc) |
| Atp2b4 | ATPase, Ca++ transporting, plasma | Capza2 | capping protein (actin filament) muscle Z-line, |
| | membrane 4(Atp2b4) | | alpha 2(Capza2) |
| Dusp26 | dual specificity phosphatase 26 | Sesn1 | sestrin 1(Sesn1) |
| | (putative)(Dusp26) | | |
| Abcb9 | ATP-binding cassette, sub-family B | Zbtb38 | zinc finger and BTB domain containing |
| | (MDR/TAP), member 9(Abcb9) | | 38(Zbtb38) |
| Tuba4a | tubulin, alpha 4A(Tuba4a) | Ppp1r15a | protein phosphatase 1, regulatory (inhibitor) |
| | | | subunit 15A(Ppp1r15a) |
| Rom1 | rod outer segment membrane protein | Nnt | nicotinamide nucleotide transhydrogenase(Nnt) |
| | 1(Rom1) | | |
| Sirt3 | sirtuin 3(Sirt3) | Ypel3 | yippee-like 3 (Drosophila)(Ypel3) |
| Plbd1 | phospholipase B domain containing | Rhno1 | RAD9-HUS1-RAD1 interacting nuclear orphan |
| | 1(Plbd1) | | 1(Rhno1) |
| Ctsc | cathepsin C(Ctsc) | Mrps36 | mitochondrial ribosomal protein S36(Mrps36) |
| Inha | inhibin alpha(Inha) | Fam234a | family with sequence similarity 234, member |
| | | | A(Fam234a) |
| Camk2a | calcium/calmodulin-dependent protein | 2310001H17Rik | RIKEN cDNA 2310001H17 |
| | kinase II alpha(Camk2a) | | gene(2310001H17Rik) |
| Synm | synemin, intermediate filament | Ksr1 | kinase suppressor of ras 1(Ksr1) |
| | protein(Synm) | | |
| Hivep3 | human immunodeficiency virus type I | Tcp11l2 | t-complex 11 (mouse) like 2(Tcp11l2) |
| | enhancer binding protein 3(Hivep3) | | |
| Pecam1 | platelet/endothelial cell adhesion molecule | B4galt1 | UDP-Gal:betaGlcNAc beta 1,4- |
| | 1(Pecam1) | | galactosyltransferase, polypeptide 1 (B4galt1) |
| Tbrg4 | transforming growth factor beta regulated | Wnk1 | WNK lysine deficient protein kinase 1(Wnk1) |
| | gene 4(Tbrg4) | | |
| Chid1 | chitinase domain containing 1(Chid1) | Hccs | holocytochrome c synthetase(Hccs) |
| Gm14698 | | Zranb2 | zinc finger, RAN-binding domain containing |
| | | | 2(Zranb2) |
| Mrm1 | mitochondrial rRNA methyltransferase | Atp2a3 | ATPase, Ca++ transporting, ubiquitous(Atp2a3) |
| | 1(Mrm1) | | |
| Calm2 | calmodulin 2(Calm2) | Mapk12 | mitogen-activated protein kinase 12(Mapk12) |
| Nrap | nebulin-related anchoring protein(Nrap) | Smtnl2 | smoothelin-like 2(Smtnl2) |
| Acaa2 | acetyl-Coenzyme A acyltransferase 2 | Mybpc1 | myosin binding protein C, slow-type(Mybpc1) |
| | (mitochondrial 3-oxoacyl-Coenzyme A | | |
| | thiolase)(Acaa2) | | |
| Gys1 | glycogen synthase 1, muscle(Gys1) | Abcd2 | ATP-binding cassette, sub-family D (ALD), |
| | | | member 2(Abcd2) |
| Plekhh3 | pleckstrin homology domain containing, | Mb21d2 | Mab-21 domain containing 2(Mb21d2) |
| | family H (with MyTH4 domain) member | | |
| | 3(Plekhh3) | | |
| Atp5g3 | ATP synthase, H+transporting, | Tmem25 | transmembrane protein 25(Tmem25) |
| | mitochondrial F0 complex, subunit C3 | | |
| | (subunit 9)(Atp5g3) | | |
| Lrp4 | low density lipoprotein receptor-related | Abca9 | ATP-bindingcassette, sub-family A (ABC1), |
| | protein 4(Lrp4) | | member 9(Abca9) |
| Mrpl47 | mitochondrial ribosomal protein | Tgfb2 | transforming growth factor, beta 2(Tgfb2) |
| | L47(Mrpl47) | | |
| Coq10a | coenzyme Q10A(Coq10a) | Fam210a | family with sequence similarity 210, member |
| | | | A(Fam210a) |
| Mb | myoglobin(Mb) | Lace1 | lactation elevated 1(Lace1) |
| Sccpdh | saccharopine dehydrogenase | Pigp | phosphatidylinositol glycan anchor biosynthesis, |
| | (putative)(Sccpdh) | | class P(Pigp) |
| Khdrbs3 | KH domain containing, RNA binding, | Fgd4 | FYVE, RhoGEF and PH domain containing |
| | signal transduction associated 3(Khdrbs3) | | 4(Fgd4) |
| Dhrs7c | dehydrogenase/reductase (SDR family) | Htatsf1 | HIV TAT specific factor 1(Htatsf1) |
| | member 7C(Dhrs7c) | | |
| Cebpb | CCAAT/enhancer binding protein | Ablim1 | actin-binding LIM protein 1(Ablim1) |
| | (C/EBP), beta(Cebpb) | | |
| Epb41l4b | erythrocyte membrane protein band 4.1 | Cul3 | cullin 3(Cul3) |
| | like 4b(Epb41l4b) | | |
| Txnrd1 | thioredoxin reductase 1(Txnrd1) | Acta2 | actin, alpha 2, smooth muscle, aorta(Acta2) |
| Sel1l3 | sel-1 suppressor of lin-12-like 3 (C. | Scn4a | sodium channel, voltage-gated, type IV, |
| | elegans)(Sel1l3) | | alpha(Scn4a) |
| Ar | androgen receptor(Ar) | Kcnab1 | potassium voltage-gated channel, shaker-related |
| | | | subfamily, beta member 1(Kcnab1) |
| Pam | peptidylglycine alpha-amidating | Acadsb | acyl-Coenzyme A dehydrogenase, |
| | monooxygenase(Pam) | | short/branched chain(Acadsb) |
| Dcun1d5 | DCN1, defective in cullin neddylation 1, | Adipor2 | adiponectin receptor 2(Adipor2) |
| | domain containing 5 (S. | | |
| | cerevisiae)(Dcun1d5) | | |
| Dcakd | dephospho-CoA kinase domain | Kremen1 | kringle containing transmembrane protein |
| | containing(Dcakd) | | 1(Kremen1) |
| Cdk18 | cyclin-dependent kinase 18(Cdk18) | Glb1l2 | galactosidase, beta l-like 2(Glb1l2) |
| Pias1 | protein inhibitor of activated STAT | Pabpc1 | poly(A) binding protein, cytoplasmic 1(Pabpc1) |
| | 1(Pias1) | | |
| Phc1 | polyhomeotic-like 1 (Drosophila)(Phc1) | Eif3a | eukaryotic translation initiation factor 3, subunit |
| | | | A(Eif3a) |
| Klhl24 | kelch-like 24(Klhl24) | Rhou | ras homolog family member U(Rhou) |
| Slc38a4 | solute carrier family 38, member | Tet2 | tet methylcytosine dioxygenase 2(Tet2) |
| | 4(Slc38a4) | | |
| Nhs | Nance-Horan syndrome (human)(Nhs) | Cog6 | component of oligomeric golgi complex |
| | | | 6(Cog6) |
| Gatad2b | GATA zinc finger domain containing | Ccdc80 | coiled-coil domain containing 80(Ccdc80) |
| | 2B(Gatad2b) | | |
| Tnfrsf21 | tumor necrosis factor receptor superfamily, | Cox7a1 | cytochrome c oxidase subunit VIIa 1(Cox7a1) |
| | member 21(Tnfrsf21) | | |
| Cited4 | Cbp/p300-interacting transactivator, with | Slmo2 | slowmo homolog 2 (Drosophila)(Slmo2) |
| | Glu/Asp-rich carboxy-terminal domain, | | |
| | 4(Cited4) | | |
| Ltbp4 | latent transforming growth factor beta | Adcy9 | adenylate cyclase 9(Adcy9) |
| | binding protein 4(Ltbp4) | | |
| 0610012G03Rik | RIKEN cDNA 0610012G03 | Atad1 | ATPase family, AAA domain containing |
| | gene(0610012G03Rik) | | 1(Atad1) |

In addition to its ability to bind T₃, μ-crystallin also is a ketimine reductase, with its activity inhibited by T₃ binding. Although this activity is likely inhibited by the high intracellular levels of T₃ in Crym tg muscle, five genes involved in lysine degradation (a pathway including substrates that μ-crystallin acts on in its capacity as a ketimine reductase), compared to controls, with Aldh2, Ogdh and Dist increasing in expression and Sccpdh and Setd7 decreasing in expression.

TABLE 10
Significant Biological GO Terms

| | | FDR |
|---|---|---|
| | Fold | adj. p |
| GO biological process complete | Enrichment | Value |
| sensory perception of smell (GO: 0007608) | <0.01 | 1.81E−09 |
| Unclassified (UNCLASSIFIED) | 0.32 | 2.46E−05 |
| G-protein coupled receptor signaling pathway (GO: 0007186) | 0.35 | 6.14E−05 |
| tricarboxylic acid cycle (GO: 0006099) | 12.19 | 6.47E−05 |
| glucose metabolic process (GO: 0006006) | 6.04 | 7.16E−05 |
| pyruvate metabolic process (GO: 0006090) | 7.2 | 2.43E−04 |
| 2-oxoglutarate metabolic process (GO: 0006103) | 14.47 | 3.98E−04 |
| cellular response to oxygen-containing compound (GO: 1901701) | 2.17 | 7.89E−04 |
| NADPH oxidation (GO: 0070995) | 28.04 | 9.17E−04 |
| glycogen biosynthetic process (GO: 0005978) | 13.86 | 2.02E−03 |
| monocarboxylic acid biosynthetic process (GO: 0072330) | 3.72 | 2.02E−03 |
| striated muscle contraction (GO: 0006941) | 4.71 | 2.61E−03 |
| regulation of cellular component organization (GO: 0051128) | 1.56 | 2.79E−03 |
| fatty acid metabolic process (GO: 0006631) | 2.9 | 3.11E−03 |
| regulation of actin filament-based process (GO: 0032970) | 2.58 | 3.48E−03 |
| response to mechanical stimulus (GO: 0009612) | 3.9 | 3.51E−03 |
| response to hypoxia (GO: 0001666) | 3.47 | 3.66E−03 |
| positive regulation of small molecule metabolic process (GO: 0062013) | 3.41 | 4.28E−03 |
| carbohydrate catabolic process (GO: 0016052) | 5.24 | 4.74E−03 |
| negative regulation of cell death (GO: 0060548) | 1.86 | 4.98E−03 |
| oxaloacetate metabolic process (GO: 0006107) | 14.02 | 7.47E−03 |
| regulation of glycolytic process (GO: 0006110) | 7.63 | 7.60E−03 |

### Example 10: Global Protein Expression (LC.MS/MS)

Proteomic analysis was performed using liquid chromatography-tandem mass spectrometry (LC.MS/MS) followed by bioinformatic assessment (see Table 11). GO analysis was performed on the 85 significantly differentially expressed proteins using the PANTHER Classification System, which resulted in 7 statistically significant ontological classes. Four of the terms related to muscle while one was for oxidation-reduction process (see Table 12). Eleven proteins that were significantly differentially expressed in Crym tg mice compared to controls were associated with the “oxidation-reduction process” gene ontology. Five of those proteins increased in expression in Crym tg mice compared to controls while 6 decreased. See Table 13. The second column lists the abundance ratio of the proteins expressed as the average abundance of Crym tg/control protein. The false discovery rate corrected p value for each protein is listed in the third column (n=6; α=0.05). Although the gene products revealed by LC.MS/MS and RNA-seq showed minimal overlap, the significant ontological terms in the proteomic study were similar to those found in the transcriptomic study.

TABLE 11
Significantly Differentially Expressed Proteins in Crym tg Muscle

| | | Abundance | Abundance |
|---|---|---|---|
| | | Ratio: | Ratio Adj. P- |
| | | (Crym)/ | Value: |
| Accession | Description | (BL6) | (Crym)/(BL6) |
| Q8K339 | DNA/RNA-binding protein KIN17 OS = Mus | 1000 | 5.06415E−16 |
| | musculus GN = Kin PE = 1 SV = 1 | | |
| Q8CG76 | Aflatoxin B1 aldehyde reductase member 2 | 1000 | 5.06415E−16 |
| | OS = Mus musculus GN = Akr7a2 PE = 1 SV = 3 | | |
| Q3TJZ6 | Protein FAM98A OS = Mus musculus | 1000 | 5.06415E−16 |
| | GN = Fam98a PE = 1 SV = 1 | | |
| Q8QZR5 | Alanine aminotransferase 1 OS = Mus musculus | 1000 | 5.06415E−16 |
| | GN = Gpt PE = 1 SV = 3 | | |
| P00416 | Cytochrome c oxidase subunit 3 OS = Mus | 1000 | 5.06415E−16 |
| | musculus GN = mt-Co3 PE = 1 SV = 2 | | |
| P06728 | Apolipoprotein A-IV OS = Mus musculus | 1000 | 5.06415E−16 |
| | GN = Apoa4 PE = 1 SV = 3 | | |
| Q99PE2 | Ankyrin repeat family A protein 2 OS = Mus | 1000 | 5.06415E−16 |
| | musculus GN = Ankra2 PE = 1 SV = 1 | | |
| Q8BP92 | Reticulocalbin-2 OS = Mus musculus GN = Rcn2 | 1000 | 5.06415E−16 |
| | PE = 1 SV = 1 | | |
| P24527 | Leukotriene A-4 hydrolase OS = Mus musculus | 1000 | 5.06415E−16 |
| | GN = Lta4h PE = 1 SV = 4 | | |
| E9Q1Y9 | Keratin 83 OS = Mus musculus GN = Krt83 PE = 1 | 0.001 | 5.06415E−16 |
| | SV = 1 | | |
| Q9DCF9 | Translocon-associated protein subunit gamma | 0.001 | 5.06415E−16 |
| | OS = Mus musculus GN = Ssr3 PE = 1 SV = 1 | | |
| Q61897 | Keratin, type I cuticular Ha3-II OS = Mus | 0.001 | 5.06415E−16 |
| | musculus GN = Krt33b PE = 1 SV = 2 | | |
| P16627 | Heat shock 70 kDa protein l-like OS = Mus | 0.001 | 5.06415E−16 |
| | musculus GN = Hspa1l PE = 1 SV = 4 | | |
| Q9D638 | Keratin-associated protein 3-2 OS = Mus musculus | 0.001 | 5.06415E−16 |
| | GN = Krtap3-2 PE = 3 SV = 2 | | |
| Q5FW53 | Myosin-binding protein H-like OS = Mus musculus | 0.001 | 5.06415E−16 |
| | GN = Mybphl PE = 1 SV = 1 | | |
| Q8VDQ1 | Prostaglandin reductase 2 OS = Mus musculus | 0.001 | 5.06415E−16 |
| | GN = Ptgr2 PE = 1 SV = 2 | | |
| A0A140T8T7 | Collagen alpha-5(VI) chain OS = Mus musculus | 0.001 | 5.06415E−16 |
| | GN = Col6a5 PE = 1 SV = 1 | | |
| O54983 | Ketimine reductase mu-crystallin OS = Mus | 13.205 | 5.06415E−16 |
| | musculus GN = Crym PE = 1 SV = 1 | | |
| A3KGU9 | Spectrin alpha chain, non-erythrocytic 1 OS = Mus | 0.304 | 5.06415E−16 |
| | musculus GN = Sptanl PE = 1 SV = 2 | | |
| Q80XN0 | D-beta-hydroxybutyrate dehydrogenase, | 0.314 | 5.06415E−16 |
| | mitochondrial OS = Mus musculus GN = Bdhl | | |
| | PE = 1 SV = 2 | | |
| Q91V41 | Ras-related protein Rab-14 OS = Mus musculus | 0.352 | 5.06415E−16 |
| | GN = Rab14 PE = 1 SV = 3 | | |
| E9Q8Y0 | Myosin regulatory light chain 2, | 2.672 | 5.06415E−16 |
| | ventricular/cardiac muscle isoform OS = Mus | | |
| | musculus GN = Myl2 PE = 1 SV = 1 | | |
| P50462 | Cysteine and glycine-rich protein 3 OS = Mus | 0.435 | 5.06415E−16 |
| | musculus GN = Csrp3 PE = 1 SV = 1 | | |
| Q9WV06 | Ankyrin repeat domain-containing protein 2 | 0.388 | 3.08626E−12 |
| | OS = Mus musculus GN = Ankrd2 PE = 1 SV = 3 | | |
| P21812 | Mast cell protease 4 OS = Mus musculus | 2.127 | 3.08626E−12 |
| | GN = Mcpt4 PE = 1 SV = 1 | | |
| P29391 | Ferritin light chain 1 OS = Mus musculus GN = Ftl1 | 0.531 | 1.76191E−09 |
| | PE = 1 SV = 2 | | |
| P97861 | Keratin, type II cuticular Hb6 OS = Mus musculus | 0.541 | 6.35461E−09 |
| | GN = Krt86 PE = 2 SV = 2 | | |
| Q61033 | Lamina-associated polypeptide 2, isoforms | 2.298 | 1.32401E−08 |
| | alpha/zeta OS = Mus musculus GN = Tmpo PE = 1 | | |
| | SV = 4 | | |
| P70695 | Fructose-1,6-bisphosphatase isozyme 2 OS = Mus | 1.724 | 9.85824E−08 |
| | musculus GN = Fbp2 PE = 1 SV = 2 | | |
| A0A087WRF2 | Arf-GAP with GTPase, ANK repeat and PH | 0.565 | 1.12072E−07 |
| | domain-containing protein 1 OS = Mus musculus | | |
| | GN = Agap1 PE = 1 SV = 1 | | |
| Q9DCL9 | Multifunctional protein ADE2 OS = Mus musculus | 0.427 | 1.68913E−07 |
| | GN = Paics PE = 1 SV = 4 | | |
| Q640L5 | Coiled-coil domain-containing protein 18 | 1.689 | 3.43509E−07 |
| | OS = Mus musculus GN = Ccdc18 PE = 1 SV = 1 | | |
| P23927 | Alpha-crystallin B chain OS = Mus musculus | 0.576 | 3.48308E−07 |
| | GN = Cryab PE = 1 SV = 2 | | |
| Q3U381 | Zinc finger protein 692 OS = Mus musculus | 0.508 | 2.38708E−06 |
| | GN = Znf692 PE = 2 SV = 1 | | |
| Q9D8B3 | Charged multivesicular body protein 4b OS = Mus | 0.539 | 3.0525E−05 |
| | musculus GN = Chmp4b PE = 1 SV = 2 | | |
| Q05722 | Collagen alpha-1(IX) chain OS = Mus musculus | 0.55 | 3.7709E−05 |
| | GN = Col9a1 PE = 2 SV = 2 | | |
| Q8JZN5 | Acyl-CoA dehydrogenase family member 9, | 1.574 | 5.20719E−05 |
| | mitochondrial OS = Mus musculus GN = Acad9 | | |
| | PE = 1 SV = 2 | | |
| F6ZDS4 | Nucleoprotein TPR OS = Mus musculus GN = Tpr | 1.526 | 9.5752E−05 |
| | PE = 1 SV = 1 | | |
| F6ZHD8 | 1,4-alpha-glucan-branching enzyme OS = Mus | 0.639 | 0.000109695 |
| | musculus GN = Gbe1 PE = 1 SV = 2 | | |
| P00329 | Alcohol dehydrogenase 1 OS = Mus musculus | 0.577 | 0.000163926 |
| | GN = Adh1 PE = 1 SV = 2 | | |
| Q6W8Q3 | Purkinje cell protein 4-like protein 1 OS = Mus | 0.511 | 0.000166742 |
| | musculus GN = Pcp4l1 PE = 1 SV = 1 | | |
| A0A1Y7VM56 | NAD-dependent protein deacylase sirtuin-5, | 1.773 | 0.000270852 |
| | mitochondrial OS = Mus musculus GN = Sirt5 PE = 1 | | |
| | SV = 1 | | |
| Q4VAE3 | Transmembrane protein 65 OS = Mus musculus | 0.563 | 0.000503491 |
| | GN = Tmem65 PE = 1 SV = 1 | | |
| P26039 | Talin-1 OS = Mus musculus GN = Tln1 PE = 1 SV = 2 | 0.54 | 0.00078635 |
| A2AEX6 | Four and a half LIM domains protein 1 OS = Mus | 0.672 | 0.001101028 |
| | musculus GN = Fhl1 PE = 1 SV = 1 | | |
| O35367 | Keratocan OS = Mus musculus GN = Kera PE = 2 | 1.445 | 0.00111612 |
| | SV = 1 | | |
| Q8OVH0 | B-cell scaffold protein with ankyrin repeats | 0.546 | 0.001363127 |
| | OS = Mus musculus GN = Bank1 PE = 1 SV = 3 | | |
| A0A0R4J0J0 | Palmdelphin OS = Mus musculus GN = Palmd PE = 1 | 0.676 | 0.001363127 |
| | SV = 1 | | |
| Q8VCT4 | Carboxylesterase 1D OS = Mus musculus | 1.538 | 0.00136542 |
| | GN = Cesld PE = 1 SV = 1 | | |
| Q9QXT0 | Protein canopy homolog 2 OS = Mus musculus | 1.634 | 0.002602884 |
| | GN = Cnpy2 PE = 1 SV = 1 | | |
| E9QQ57 | Periaxin OS = Mus musculus GN = Prx PE = 1 SV = 1 | 0.656 | 0.002609724 |
| Q9JMC3 | DnaJ homolog subfamily A member 4 OS = Mus | 0.605 | 0.002781199 |
| | musculus GN = Dnaja4 PE = 1 SV = 1 | | |
| A0A0A6YVU8 | MCG119397 OS = Mus musculus GN = Gm9774 | | |
| | PE = 4 SV = 1 | 0.576 | 0.002855412 |
| A0A0R4J135 | Selenium-binding protein 2 OS = Mus musculus | 1.403 | 0.003127834 |
| | GN = Selenbp2 PE = 1 SV = 1 | | |
| P54071 | Isocitrate dehydrogenase [NADP], mitochondrial | 1.389 | 0.00459371 |
| | OS = Mus musculus GN = Idh2 PE = 1 SV = 3 | | |
| A2AQP0 | Myosin-7B OS = Mus musculus GN = Myh7b PE = 3 | 1.381 | 0.005681633 |
| | SV = 1 | | |
| A2AT68 | Titin (Fragment) OS = Mus musculus GN = Ttn | 1.381 | 0.005681633 |
| | PE = 1 SV = 1 | | |
| Q9CZB0 | Succinate dehydrogenase cytochrome b560 | 0.705 | 0.006105325 |
| | subunit, mitochondrial OS = Mus musculus | | |
| | GN = Sdhc PE = 1 SV = 1 | | |
| A0A0R4J0I1 | MCG1051009 OS = Mus musculus GN = Serpina3k | 0.707 | 0.006580056 |
| | PE = 1 SV = 1 | | |
| Q9D0M5 | Dynein light chain 2, cytoplasmic OS = Mus | 0.709 | 0.007407379 |
| | musculus GN = Dynll2 PE = 1 SV = 1 | | |
| Q5EBG6 | Heat shock protein beta-6 OS = Mus musculus | 0.711 | 0.008191236 |
| | GN = Hspb6 PE = 1 SV = 1 | | |
| P30412 | Peptidyl-prolyl cis-trans isomerase C OS = Mus | 0.616 | 0.008459428 |
| | musculus GN = Ppic PE = 1 SV = 1 | | |
| F6QYE1 | Calsequestrin OS = Mus musculus GN = Casq2 | 1.363 | 0.008616749 |
| | PE = 1 SV = 1 | | |
| P04938 | Major urinary protein 11 OS = Mus musculus | 0.61 | 0.008847624 |
| | GN = Mup11 PE = 1 SV = 2 | | |
| P16045 | Galectin-1 OS = Mus musculus GN = Lgals1 PE = 1 | 0.715 | 0.008847624 |
| | SV = 3 | | |
| P15626 | Glutathione S-transferase Mu 2 OS = Mus | 1.351 | 0.010822634 |
| | musculus GN = Gstm2 PE = 1 SV = 2 | | |
| Q60668 | Heterogeneous nuclear ribonucleoprotein D0 | 0.72 | 0.010964372 |
| | OS = Mus musculus GN = Hnrnpd PE = 1 SV = 2 | | |
| P46656 | Adrenodoxin, mitochondrial OS = Mus musculus | 1.466 | 0.011591329 |
| | GN = Fdx1 PE = 1 SV = 1 | | |
| P21981 | Protein-glutamine gamma-glutamyltransferase 2 | 0.722 | 0.011862729 |
| | OS = Mus musculus GN = Tgm2 PE = 1 SV = 4 | | |
| Q80WJ7 | Protein LYRIC OS = Mus musculus GN = Mtdh | 1.484 | 0.016201938 |
| | PE = 1 SV = 1 | | |
| P14602 | Heat shock protein beta-1 OS = Mus musculus | 0.732 | 0.01962496 |
| | GN = Hspb1 PE = 1 SV = 3 | | |
| F8WIV2 | Serine (or cysteine) peptidase inhibitor, clade B, | 0.733 | 0.019861099 |
| | member 6a OS = Mus musculus GN = Serpinb6a | | |
| | PE = 1 SV = 1 | | |
| Q9D358 | Low molecular weight phosphotyrosine protein | 1.322 | 0.022273123 |
| | phosphatase OS = Mus musculus GN = Acp1 PE = 1 | | |
| | SV = 3 | | |
| D3Z6H8 | S-adenosylmethionine decarboxylase proenzyme | 0.625 | 0.02364725 |
| | 2 OS = Mus musculus GN = Amd2 PE = 1 SV = 1 | | |
| Q9DCD0 | 6-phosphogluconate dehydrogenase, | 0.625 | 0.02432232 |
| | decarboxylating OS = Mus musculus GN = Pgd | | |
| | PE = 1 SV = 3 | | |
| P14148 | 60S ribosomal protein L7 OS = Mus musculus | 1.386 | 0.024879354 |
| | GN = Rpl7 PE = 1 SV = 2 | | |
| P14069 | Protein S100-A6 OS = Mus musculus GN = S100a6 | 0.741 | 0.027720305 |
| | PE = 1 SV = 3 | | |
| Q04447 | Creatine kinase B-type OS = Mus musculus | 0.631 | 0.030605959 |
| | GN = Ckb PE = 1 SV = 1 | | |
| P62962 | Profilin-1 OS = Mus musculus GN = Pfnl PE = 1 | 0.747 | 0.030745961 |
| | SV = 2 | | |
| P20152 | Vimentin OS = Mus musculus GN = Vim PE = 1 | 0.748 | 0.031738217 |
| | SV = 3 | | |
| P97927 | Laminin subunit alpha-4 OS = Mus musculus | 0.637 | 0.03202606 |
| | GN = Lama4 PE = 1 SV = 2 | | |
| A2CG35 | Ras-related protein Rab-12 OS = Mus musculus | 1.413 | 0.033333527 |
| | GN = Rab12 PE = 1 SV = 1 | | |
| E9PXQ7 | Protocadherin 10 OS = Mus musculus GN = Pcdh10 | 0.752 | 0.036907598 |
| | PE = 1 SV = 1 | | |
| Q3UIU2 | NADH dehydrogenase [ubiquinone] 1 beta | 0.754 | 0.039259058 |
| | subcomplex subunit 6 OS = Mus musculus | | |
| | GN = Ndufb6 PE = 1 SV = 3 | | |
| Q9DA97 | Septin-14 OS = Mus musculus GN = Sept14 PE = 1 | 1.304 | 0.042479617 |
| | SV = 3 | | |

TABLE 12
Effects on glycolytic and oxidative patheway genes.

| Glycolytic Pathway | | Oxidative Pathway | | Fast Contractile | | Slow Contractile | |
|---|---|---|---|---|---|---|---|
| Genes | | Genes | | Genes | | Genes | |
| # | # | # | # | # | # | # | # |
| increased | decreased | increased | decreased | increased | decreased | increased | decreased |
| 5 | 11 | 29 | 20 | 10 | 43 | 54 | 8 |

TABLE 13
Proteins Involved in Oxidation-Reduction Process Differentially
Expressed in Crym tg TA Muscle.

| | Abundance Ratio: | Abundance Ratio Adj. |
|---|---|---|
| | (Crym tg)/ | P-Value: (Crym tg) / |
| Gene | (control) | (control) |
| Akr7a2 | 1000 | 5.064E−16 |
| Crym | 13.205 | 5.064E−16 |
| Bdh1 | 0.314 | 5.064E−16 |
| Ptgr2 | 0.001 | 5.064E−16 |
| Acad9 | 1.574 | 5.207E−05 |
| Adh1 | 0.577 | 1.639E−04 |
| Idh2 | 1.389 | 4.594E−03 |
| Sdhc | 0.705 | 6.105E−03 |
| Fdx1 | 1.466 | 1.159E−02 |
| Pgd | 0.625 | 2.432E−02 |
| Ndufb6 | 0.754 | 3.926E−02 |

### Example 11: Method of Treatment

AAV or small molecules are delivered to patients at appropriate doses and at a given dosing regimen as determined by the practitioner. Subject inclusion criteria include individuals with lipolytic disorders, lipogenic disorders, glycolytic disorders, gluconeogenic disorders, type 2 diabetes, obesity, or any combination thereof. Individuals are human patients and preferably do not have other major underlying health disorders. The patients will primarily be adults of any age, preferably from about 18 to about 65 years of age. The dose of AAV or of a small molecule may be given one to three times and may be separated by one week up to two month in between doses.

### REFERENCES

All references listed below and throughout the specification are hereby incorporated by reference in their entirety.
1. Abe S et al. Identification of CRYM as a candidate responsible for nonsyndromic deafness, through cDNA microarray analysis of human cochlear and vestibular tissues. The American Journal of Human Genetics 72: 73-82, 2003.
2. Aguet F et al., Model-based 2.5-D deconvolution for extended depth of field in brightfield microscopy. IEEE Transactions on Image Processing 17: 1144-1153, 2008.
3. Anion D et al., Molecular evidence for increased expression of genes related to immune and chaperone function in the prefrontal cortex in schizophrenia. Biological psychiatry 62: 711-721, 2007.
4. Ashburner M et al., Gene ontology: tool for the unification of biology. Nature genetics, 2000.25(1): p. 25.
5. Augusto V et al., Skeletal muscle fiber types in C57BL6J mice. Braz J Morphol Sci, 2004. 21(2): p. 89-94.
6. Bancroft J D and Gamble M, Theory and practice of histological techniques. Elsevier health sciences, 2008.
7. Beslin A et al., Identification by photoaffinity labelling of a pyridine nucleotide-dependent tri-iodothyronine-binding protein in the cytosol of cultured astroglial cells. Biochemical Journal 305: 729-737, 1995.
8. Bianco A C and Kim B W, Deiodinases: implications of the local control of thyroid hormone action. The Journal of clinical investigation 116: 2571-2579, 2006.
9. Bishopric N H and Kedes L, Adrenergic regulation of the skeletal alpha-actin gene promoter during myocardial cell hypertrophy. Proceedings of the National Academy of Sciences 88: 2132-2136, 1991.
10. Borel F et al., Crystal structure of mouse mu-crystallin complexed with NADPH and the T₃ thyroid hormone. The FEBS Journal 281: 1598-1612, 2014.
11. Briguet A et al., Histological parameters for the quantitative assessment of muscular dystrophy in the mdx-mouse. Neuromuscular disorders 14: 675-682, 2004.
12. Brooke M H and Kaiser K K, Three “myosin adenosine triphosphatase” systems: the nature of their pH lability and sulfhydryl dependence. Journal of Histochemistry & Cytochemistry 18: 670-672, 1970.
13. Burks T N et al., Losartan restores skeletal muscle remodeling and protects against disuse atrophy in sarcopenia. Science translational medicine 3: 82ra37-82ra37, 2011.
14. Chen X et al., The number of x chromosomes causes sex differences in adiposity in mice. PLoS Genet 8: e1002709, 2012.
15. Ciciliot S et al., Muscle type and fiber type specificity in muscle wasting. The international journal of biochemistry & cell biology 45: 2191-2199, 2013.
16. Clement K et al., In vivo regulation of human skeletal muscle gene expression by thyroid hormone. Genome research 12: 281-291, 2002.
17. Collie E and Muscat G E, The human skeletal alpha-actin promoter is regulated by thyroid hormone: identification of a thyroid hormone response element. Cell growth & differentiation: the molecular biology journal of the American Association for Cancer Research 3: 31-42, 1992.
18. Consortium GO, The Gene Ontology resource: 20 years and still GOing strong. Nucleic Acids Research, 2018. 47(D1): p. D330-D338.
19. Dixit M et al., DUX4, a candidate gene of facioscapulohumeral muscular dystrophy, encodes a transcriptional activator of PITX1. Proceedings of the National Academy of Sciences 104: 18157-18162, 2007.
20. Dorfer V et al., M S Amanda, a universal identification algorithm optimized for high accuracy tandem mass spectra. Journal of proteome research 13: 3679-3684, 2014.
21. Drexler H C et al., On marathons and Sprints: an integrated quantitative proteomics and transcriptomics analysis of differences between slow and fast muscle fibers. Molecular & Cellular Proteomics 11: M111. 010801, 2012.
22. Dubowitz V et al., Muscle biopsy: a practical approach: expert consult; online and print. Elsevier Health Sciences, 2013.
23. Ebashi S et al., Control of muscle contraction. Quarterly reviews of biophysics 2: 351-384, 1969.
24. Encarnacion-Rivera L et al., Myosoft: An automated muscle histology analysis tool using machine learning algorithm utilizing FIJI/ImageJ software. PloS one 15: e0229041, 2020.
25. Eng J K et al., A fast SEQUEST cross correlation algorithm. Journal of proteome research 7: 4598-4602, 2008.
26. Fitts R H et al., Contractile and fatigue properties of thyrotoxic rat skeletal muscle. Muscle & Nerve: Official Journal of the American Association of Electrodiagnostic Medicine 7: 470-477, 1984.
27. Friesema E C et al., Effective cellular uptake and efflux of thyroid hormone by human monocarboxylate transporter 10. Molecular endocrinology 22: 1357-1369, 2008.
28. Gagne R et al., Identification of thyroid hormone receptor binding sites in developing mouse cerebellum. BMC genomics 14: 341, 2013.
29. Giger J M et al., Rapid muscle atrophy response to unloading: pretranslational processes involving MHC and actin. Journal of Applied Physiology 107: 1204-1212, 2009.
30. Gunning P et al., Alpha-skeletal and alpha-cardiac actin genes are coexpressed in adult human skeletal muscle and heart. Molecular and cellular biology 3: 1985-1995, 1983.
31. Gustafson T A et al., Effects of thyroid hormone on alpha-actin and myosin heavy chain gene expression in cardiac and skeletal muscles of the rat: measurement of mRNA content using synthetic oligonucleotide probes. Circulation research 59: 194-201, 1986.
32. Hallen A and Cooper A J, Reciprocal control of thyroid binding and the pipecolate pathway in the brain. Neurochemical research 42: 217-243, 2017.
33. Hallen A et al., Mammalian forebrain ketimine reductase identified as μ-crystallin; potential regulation by thyroid hormones. Journal of neurochemistry 118: 379-387, 2011.
34. Hallen A et al., Insights into enzyme catalysis and thyroid hormone regulation of cerebral ketimine reductase/μ-crystallin under physiological conditions. Neurochemical research 40: 1252-1266, 2015.
35. Hashizume K et al., Evidence for the presence of two active forms of cytosolic 3, 5, 3′-triiodo-L-thyronine (T₃)-binding protein (CTBP) in rat kidney. Specialized functions of two CTBPs in intracellular T₃ translocation. Journal of Biological Chemistry 264: 4864-4871, 1989.
36. Homma S et al., A unique library of myogenic cells from facioscapulohumeral muscular dystrophy subjects and unaffected relatives: family, disease and cell function. European Journal of Human Genetics 20: 404-410, 2012.
37. Huang D W et al., Systematic and integrative analysis of large gene lists using DAVID bioinformatics resources. Nature protocols, 2008. 4(1): 44.
38. Huang D W et al., Bioinformatics enrichment tools: paths toward the comprehensive functional analysis of large gene lists. Nucleic acids research, 2008. 37(1):1-13.
39. Johansson C et al., Isometric force and endurance in skeletal muscle of mice devoid of all known thyroid hormone receptors. The Journal of physiology 547: 789-796, 2003.
40. Kalmar B et al., Determination of muscle fiber type in rodents. Current protocols in mouse biology 2: 231-243, 2012.
41. Kammoun M et al., A simplified immunohistochemical classification of skeletal muscle fibres in mouse. European journal of histochemistry: EJH 58: 2014.
42. Kim D et al., Noncoding dsRNA induces retinoic acid synthesis to stimulate hair follicle regeneration via TLR3. Nature Communications 10: 2811, 2019.
43. Kim R Y et al., mu-crystallin is a mammalian homologue of Agrobacterium ornithine cyclodeaminase and is expressed in human retina. Proceedings of the National Academy of Sciences 89: 9292-9296, 1992.
44. Klooster R et al., Comprehensive expression analysis of FSHD candidate genes at the mRNA and protein level. European Journal of Human Genetics 17: 1615, 2009.
45. Kobayashi M et al., A Novel NADPH-Dependent Cytosolic 3, 5, 3′-Triiodo-LThyronine-Binding Protein (CTBP; 5. IS) in Rat Liver: A Comparison with 4.7 S NADPH-Dependent CTBP. Endocrinology 129: 1701-1708, 1991.
46. Laemmli U K, Cleavage of structural proteins during the assembly of the head of bacteriophage T₄. Nature 227: 680-685, 1970.
47. Larsen P R et al., Relationships between circulating and intracellular thyroid hormones: physiological and clinical implications. Endocrine Reviews 2: 87-102, 1981.
48. Lassche S et al., Sarcomeric dysfunction contributes to muscle weakness in facioscapulohumeral muscular dystrophy. Neurology 80: 733-737, 2013.
49. Luff A, Dynamic properties of the inferior rectus, extensor digitorum longus, diaphragm and soleus muscles of the mouse. The Journal of Physiology 313: 161-171, 1981.
50. Lukyanenko V et al., Coupling of excitation to Ca2+ release is modulated by dysferlin. The Journal of Physiology 595: 5191-5207, 2017.
51. Lusk G, Animal calorimetry twenty-fourth paper. Analysis of the oxidation of mixtures of carbohydrate and fat. Journal of Biological Chemistry 59: 41-42, 1924.
52. Martins-de-Souza D et al., Proteome analysis of the thalamus and cerebrospinal fluid reveals glycolysis dysfunction and potential biomarkers candidates for schizophrenia. Journal of psychiatric research 44: 1176-1189, 2010.
53. Mi H et al., PANTHER version 11: expanded annotation datafrom Gene Ontology and Reactome pathways, and data analysis tool enhancements. Nucleic acids research, 2016. 45(01): p. D183-D189.
54. Mori J-i et al., icotinamide adenine dinucleotide phosphate-dependent cytosolic T₃ binding protein as a regulator for T₃-mediated transactivation. Endocrinology 143: 1538-1544, 2002.
55. Motulsky H J and Brown R E, Detecting outliers when fitting data with nonlinear regression—a new method based on robust nonlinear regression and the false discovery rate. BMC bioinformatics 7: 123, 2006.
56. Ohkubo Y et al., Loss of μ-crystallin causes PPARγ activation and obesity in high-fat diet-fed mice. Biochemical and biophysical research communications 508: 914-920, 2019.
57. Olojo R O et al., Mice null for calsequestrin 1 exhibit deficits in functional performance and sarcoplasmic reticulum calcium handling. PLoS One 6: e27036, 2011.
58. Peter J B et al., Metabolic profiles of three fiber types of skeletal muscle inguinea pigs and rabbits. Biochemistry, 1972. 11(14): p. 2627-2633.
59. Pette D and Staron R S, Mammalian skeletal muscle fiber type transitions. In: International Review of Cytology Elsevier, 1997, p. 143-223.
60. Pirahanchi Y and Jialal I, Physiology, Thyroid Stimulating Hormone (TSH). In: StatPearls. Treasure Island (FL): StatPearls Publishing [interna], StatPearls Publishing LLC., 2019.
61. Reed P W et al., Abnormal expression of mu-crystallin in facioscapulohumeral muscular dystrophy. Experimental neurology 205: 583-586, 2007.
62. Reichmann H and De D V, Coordinate enzymatic activity of beta-oxidation and purine nucleotide cycle in a diversity of muscle and other organs of rat. Comparative biochemistry and physiology. B, Comparative biochemistry, 1991. 98(2-3): p. 327-331.
63. Sandler B et al., Thyroxine-thyroid hormone receptor interactions. Journal of Biological Chemistry 279: 55801-55808, 2004.
64. Schiaffino S and Reggiani C, Fiber types in mammalian skeletal muscles. Physiological reviews 91: 1447-1531, 2011.
65. Schindelin J et al., Fiji: an open-source platform for biological-image analysis. Nature methods 9: 676-682, 2012.
66. Seko D et al., μ-Crystallin controls muscle function through thyroid hormone action. The FASEB Journal 30: 1733-1740, 2015.
67. Sheng J-J and Jin J-P, TNNI1, TNNI2 and TNNI3: Evolution, regulation, and protein structure-function relationships. Gene 576: 385-394, 2016.
68. Siler T et al., Inhibition by somatostatin on the release of TSH induced in man by thyrotropin-releasing factor. The Journal of Clinical Endocrinology & Metabolism 38: 742-745, 1974.
69. Spangenburg E E et al., Use of BODIPY (493/503) to visualize intramuscular lipid droplets in skeletal muscle. Journal of Biomedicine and Biotechnology 2011: 2011.
70. Suzuki S et al., μ-Crystallin as an intracellular 3, 5, 3′-triiodothyronine holder in vivo. Molecular endocrinology 21: 885-894, 2007.
71. Tata J R, A cellular thyroxine-binding protein fraction. Biochimica et biophysica acta 28: 91-94, 1958.
72. Tawil R et al., Facioscapulohumeral dystrophy: the path to consensus on pathophysiology. Skeletal muscle 4: 12, 2014.
73. Taylor S C et al., The ultimate qPCR experiment: producing publication quality, reproducible data the first time. Trends in biotechnology 37: 761-774, 2019.
74. van Mullem A A et al., Effects of thyroid hormone transporters MCT8 and MCT10 on nuclear activity of T₃. Molecular and cellular endocrinology 437: 252-260, 2016.
75. Vanderplanck C et al., The FSHD atrophic myotube phenotype is caused by DUX4 expression. PloS one 6: e26820, 2011.
76. Xie F et al., miRDeepFinder: a miRNA analysis tool for deep sequencing of plant small RNAs. Plant molecular biology 80: 75-84, 2012.
77. Zaiontz C, Real Statistics Resource Pack software (Release 6.8). real-statistics.com: 2020.

1. A method of increasing expression of CRYM protein expression in a subject in need thereof, comprising administering a compound to the subject, wherein the compound is selected from the group consisting of (a) one or more chemical compounds that increases CRYM expression as described herein; and (b) a vector that expresses a CRYM protein.

2. A method of claim 1, wherein the vector comprises SEQ ID NO:2.

3. A method of claim 1, wherein the one or more chemical compounds that increase CRYM expression are selected from the group consisting of pentanal, tretinoin, fenretinide, estradiol 3-benzoate, dihydrotestosterone and any combination thereof.

4. A method of claim 1, wherein the subject in need suffers from a condition selected from the group consisting of a lipolytic disorder, a lipogenic disorder, a glycolytic disorder, a gluconeogenic disorder, type 2 diabetes, obesity, and a combination thereof.

5. A method of claim 4, wherein the subject suffers from obesity, type 2 diabetes, or a combination thereof.

6. A method of claim 1, wherein the administering is by intravenous, subcutaneous, or intramuscular injection.

7. A method of claim 1, wherein the administering is oral.

8. A method of shifting energy usage from glycolytic to oxidative pathways in muscle in a subject in need thereof, comprising administering a compound to the subject, wherein the compound is selected from the group consisting of (a) one or more chemical compounds that increases CRYM expression as described herein; and (b) an AAV vector that expresses a CRYM protein.

9. A method of treatment of obesity and type 2 diabetes in a subject in need thereof, comprising administering a compound to the subject, wherein the compound is selected from the group consisting of (a) one or more chemical compounds that increases CRYM expression as described herein; and (b) an AAV vector that expresses a CRYM protein.


SEQUENCE LISTING (13 sequences)

SEQ ID NO: 1 - DNA, 4669 bp, Artificial Sequence
  Synthetic
cacccacccccactcccctacccacccactctccctttttggccctggcattcccctgtactggggcatataaagtttgcaagtccaatgggcctctctttccagtgatggccgactaggccatcttttgatacatatgcagctagagtcaagagctccggggtactggttagttcataatgttgttccacctatagggttgcagatccctttagctccttgggtactttctctagctcctccattgggagccctgtatccatccattagctgactgtgagcatccacttctgtgtttgctaggccccagcatagtctcacaagagacagctacatctgggtcctttcaataaaatcttgctagtgtatgcaatggtgtcagcgtttggatgctgattatggggtggatccctggatatggcagtctctacatggtccatcctttcatctcatctccaaactttgtctctgtaactccttccatgggtgttttgttcccaattctaaggaggggcatagtgtccacacttcagtcttcattcttattgagtttcatgtgtttagcaaattgtatcttatatcttgggtatcctaggtttggggctaatatccacttatcagtgagtacatattatgtgagttcctttgtgaatgtgttacctcactcaggatgtctgggcctctgagagggtcagtgtcctgccccaacccatgagatgacagactataatagccacaggattaacatagcaggcattgtctttctctgactatagggtgggtattatgtgttcatcaaccatcctaaaaatacccggtaaacaggtgcagccccagatcatcatcgattacgtagctagcgtttaaacttaagctttctgtaaggaaaggtaagagttgaactgagcaagagttttgaaaaatagtgacaatcccattctcctttggaatgcgcacaaatattgaggtatccagtgaacggcagcaaatttcctaccttcaaggcccaaatgtaagctagtccccttacgttacatgcagctcatttgctaagtggtttttttctagtatctccactactcgctgacacaggaggacacaggatgttaaaaaggaaatacagttctgtcaattattcacttactctccaaaatacttggaagaactaaatatggaaccataggagactttatcctcaccgcatagtccctatactagtcaaactccttattttttaattgatcatttttaggaaggtagcattttattcactagaacatttttgttaatacttgtttatttttgggatgaactgccatgatgtgggctacagaggagggtcgcatatgcttccatcccccttttagagaatccacacctgtcccagttgctgggttccactaccaaaagtgaattgcaactattttaggagcacttaagcacatccgaaaaatgagtgattctgttctggcccacaccacatcactgatgtacccccttaaagcatgtccctgagttcatcacagaagactgctcctcctgtgccctccacaaggttagaactgtccttgtcttagggaaaaaggagagagagagagagagagagagagagagagagagagagagagagagagggacaggcaccaactgggtaacctctgctgacccccactctactttaccataagtagctccaaatccttctagaaaatctgaaaggcatagccccatatatcagtgatataaatagaacctgcagcaggctctggtaaatgatgactacaaggtggactgggaggcagcccggccttggcaggcatcatcctctaaatataaagatgagtttgttcagcctttgcagaaggaaaaactgccacccatcctagagtgccgcgtccttgtccccccaccccctccaatttattgggaggaaggaccagctaagcctcatctaggaagagcccctcacccatctccacctccactccaggtctagccagtcctgggttgtgacccttgtctttcagccccaggagagggacacacatagtgccaccaaagaggctgggggagggcctcagcccaccaaaacctggggccagtgcgtcctacaggaggggaaccctcaccccttcaatccctttaggagacccaagggcgctgcgcgtccctgaggcggacagctccgtgtgctcaggctttgcgcctgacaggcctatccccgggagcccccgcgcctcctccccggcgctccgccctcgcctccccccgccagttgtctatcctgcgacagctgcgcgccctccggccgccggtggccctctgtgcggtgggggaaggggtcgacgtggctcagctttttggattcagggagctcgggggtgggaagagagaaatggagttccaggggcgtaaaggagagggagttcgccttccttcccttcctgagactcaggagtgactgcttctccaatcctcccaagcccaccactccacacgactccctcttcccggtagtcgcaagtgggagtttggggatctgagcaaagaacccgaagaggagttgaaatattggaagtcagcagtcaggcaccttcccgagcgcccagggcgctcagagtggacatggttggggaggcctttgggacaggtgcggttcccggagcgcaggcgcacacatgcacccaccggcgaacgcggtgaccctcgccccaccccatcccctccggcgggcaactgggtcgggtcaggaggggcaaacccgctagggagacactccatatacggcccggcccgcgttacctgggaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtgaagcctcgcttcccctccgcggcgaccagggcccgagccgagagtagcagttgtagctacccgcccaggtagggcaggagttgggaggggacagggggacagggcactaccgaggggaacctgaaggactccggggcagaacccagtcggttcacctggtcagccccaggcctgcgccctgagcgctgtgcctcgtctccggagccacacgcgctttcaagcttggtaccatgaagcgggcgccagcgttcctgagcgcagaggaggtgcaggatcaccttcgcagctccagccttctcatcccacccctggaggccgcactggccaacttctccaaaggtcccgacggaggggtcatgcagccagtgcgcaccgtggtgcctgtagccaagcaccgaggcttcctgggagtcatgcctgcctacagtgctgctgaggatgcgctcaccaccaagttagtcaccttctatgagggccacagcaacacagcggtcccctcccatcaggcatcggtgcttctctttgatcccagcaatggctccctgctggcggtcatggatggaaatgtcataactgcaaagagaacagcagcggtgtctgccattgccacaaagctgttgaagcccccaggcagtgatgtgctgtgcatccttggagcgggggtccaggcgtacagtcactatgagatcttcacagagcagttctccttcaaggaggtgagaatgtggaaccgcaccagggaaaatgctgagaagtttgcaagcacagtgcaaggagatgttcgggtctgttcatcagtgcaggaggctgtgacaggtgctgatgtcatcatcacagtcaccatggcaacagagcccattttatttggtgaatgggtaaagccgggggctcacatcaatgctgttggagccagcaggcctgactggcgagaactggatgacgagctcatgaggcaagcggtgctgtatgtggactcccgggaggctgccctgaaggagtcaggagacgttctgttgtcaggggctgacatctttgctgagcttggagaagtgatttcaggagcgaagcctgcacactgtgagaagaccacagtgttcaaatctttggggatggcagtggaagacctggttgcagccaaattagtatatgattcttggtcatctggcaagtgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatgccctccaggtccatccatttggctaggaatttcataaattcattcttttttaatagctgagtagtactccattgtgtagatgtaccacattttctgtatccattcctctgttgaggggcatctgggttctttccagcttctggctattataaataaggctgctatgaacatagtggagcatgtgtccttcttacc

SEQ ID NO: 2 - DNA, 943 bp, Artificial Sequence
  Synthetic
atgaagcgggcgccagcgttcctgagcgcagaggaggtgcaggatcaccttcgcagctccagccttctcatcccacccctggaggccgcactggccaacttctccaaaggtcccgacggaggggtcatgcagccagtgcgcaccgtggtgcctgtagccaagcaccgaggcttcctgggagtcatgcctgcctacagtgctgctgaggatgcgctcaccaccaagttagtcaccttctatgagggccacagcaacacagcggtcccctcccatcaggcatcggtgcttctctttgatcccagcaatggctccctgctggcggtcatggatggaaatgtcataactgcaaagagaacagcagcggtgtctgccattgccacaaagctgttgaagcccccaggcagtgatgtgctgtgcatccttggagcgggggtccaggcgtacagtcactatgagatcttcacagagcagttctccttcaaggaggtgagaatgtggaaccgcaccagggaaaatgctgagaagtttgcaagcacagtgcaaggagatgttcgggtctgttcatcagtgcaggaggctgtgacaggtgctgatgtcatcatcacagtcaccatggcaacagagcccattttatttggtgaatgggtaaagccgggggctcacatcaatgctgttggagccagcaggcctgactggcgagaactggatgacgagctcatgaggcaagcggtgctgtatgtggactcccgggaggctgccctgaaggagtcaggagacgttctgttgtcaggggctgacatctttgctgagcttggagaagtgatttcaggagcgaagcctgcacactgtgagaagaccacagtgttcaaatctttggggatggcagtggaagacctggttgcagccaaattagtatatgattcttggtcatctggcaagtgag

SEQ ID NO: 3 - DNA, 22 bp, Artificial Sequence
  Synthetic
tggccacgcgtcgactagtacg

SEQ ID NO: 4 - DNA, 25 bp, Artificial Sequence
  Synthetic
aattcgtactagtcgacgcgtggcc

SEQ ID NO: 5 - DNA, 21 bp, Artificial Sequence
  Synthetic
gagatgttcgggtctgttcat

SEQ ID NO: 6 - DNA, 25 bp, Artificial Sequence
  Synthetic
  misc_feature: 5′ FAM, 3′ 3IABkFQ
  modified_base (9)..(10): ZEN
tcatcacagtcaccatggcaacaga

SEQ ID NO: 7 - DNA, 23 bp, Artificial Sequence
  Synthetic
ggctttacccattcaccaaataa

SEQ ID NO: 8 - DNA, 23 bp, Artificial Sequence
  Synthetic
taaacccagagttcacttcctac

SEQ ID NO: 9 - DNA, 27 bp, Artificial Sequence
  Synthetic
  misc_feature: 5′ 5HEX, 3′ 3IABkFQ
  modified_base (9)..(10): ZEN
tcgtcttcagtttccatcaacagtgca

SEQ ID NO: 10 - DNA, 19 bp, Artificial Sequence
  Synthetic
ccttctcgcaacattccca

SEQ ID NO: 11 - DNA, 22 bp, Artificial Sequence
  Synthetic
ctcctttcctctagggctatct

SEQ ID NO: 12 - DNA, 24 bp, Artificial Sequence
  Synthetic
  misc_feature: 5′ HEX, 3′ 3IABkFQ
  modified_base (9)..(10): ZEN
tctctgtctcccttacccacagct

SEQ ID NO: 13 - DNA, 22 bp, Artificial Sequence
  Synthetic
agtgctgacatctcattccttc